SEQUENCE LISTING (162 sequences)

SEQ ID NO: 1 - protein, 269 aa, Mus sp.
EVQLQQSGPELVKPGALVKISCKASGYTVTSYDINWVKQRPGQGLEWIGWIYPGDGSTKYNEKFKGKATLTVDKSSTTVYMQLSSLTSENSAVYFCARGGKYFDYWGQGTTLTVSTGGGGSGGGGSGGGGSALDIVMTQSPKFMSTSVGDRVSVTCKASQNVATNVAWYQQKPGQSPKPLTYSASFRSSGVPDRFTGSGSGTDFTLTISNVQSEDLAEYFCQQYNSYPYTFGGGTKLEIKAAAGAPVPYPDPLEPRGAASAWSHPQFEK

SEQ ID NO: 2 - protein, 288 aa, Mus sp.
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDSGLQQGPRRRGARVNFSYYGLDVWGRGTTVTVSSGGGGSGGGGSGGGGSAQAVLTQPSSASGTPGQRVTISCSGSNSNIGRNYVFWYQQFPGTAPKILIYRNNQRPSGVPDRFSGSKSGTSASLAISGLRSEDEADYYCASWDDSLTWVFGGGTKVTVLGAAAGAPVPYPDPLEPRGAASAWSHPQFEK

SEQ ID NO: 3 - DNA, 810 bp, Mus sp.
gaggtccagctgcaacagtctggacctgagctggtgaagcctggggctttagtgaagatatcctgcaaggcctcgggatacaccgtcacaagctacgatataaactgggtgaagcagaggcctggacagggacttgagtggattggatggatttatcctggagatggtagtactaagtacaatgagaaattcaagggcaaggccacactgactgtagacaaatcctccaccacagtctacatgcagctcagcagcctgacttctgagaactctgcagtctatttctgtgcaagaggtggtaaatactttgactactggggccaaggcaccactctcacagtgtcgacaggtggaggcggttcaggcggaggtggctctggcggtggcggaagtgcactcgacattgtgatgacacagtctccaaaattcatgtccacatcagtaggagacagggtcagcgtcacctgcaaggccagtcagaatgtggctactaatgtagcctggtatcaacagaaaccagggcaatctcctaaaccactgacttactcggcatccttccggtccagtggagtccctgatcgcttcacaggcagtggatctgggacagatttcactctcaccatcagcaatgtgcagtctgaagacttggcagagtatttctgtcagcaatataacagctatccgtacacgttcggaggggggaccaagctggaaataaaagcggccgcaggtgcgccggtgccgtatccagatccgctggaaccgcgtggggccgcaagcgcttggagccacccgcagttcgaaaaataa

SEQ ID NO: 4 - DNA, 867 bp, Mus sp.
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgcgagactcggggctacagcagggaccccgccgaagaggggcccgagtaaatttctcctactacggtctggacgtctgggggcgggggaccacggtcaccgtctcgagtggaggcggcggttcaggcggaggtggctctggcggtggcggaagtgcacaggctgtgctgactcagccgtcctcagcgtctgggacccccgggcagagggtcaccatctcttgttctggaagcaactccaacatcggacgcaattatgtattctggtaccagcagttcccaggaacggcccccaaaatcctcatctacaggaacaatcagcggccctcaggggtccctgaccgattctctggctccaagtctggcacatcagcctccctggccatcagtgggctccggtccgaggatgaggctgattattactgtgcatcatgggatgacagcctgacttgggtgttcggcggagggaccaaggtcaccgtcctaggtgcggccgcaggtgcgccggtgccgtatccagatccgctggaaccgcgtggggccgcaagcgcttggagccacccgcagttcgaaaaataa

SEQ ID NO: 5 - protein, 246 aa, Homo sapiens
ASVKVSCKTSGYTFIAYYIHWVRQAPGQGLEWMGRINPNTGGINLAQKFQGRVTVTRDTSISTAHMELSRLSSDDTAVYYCARERIVPAGLRNRGMVTAVGMDVWGRGTLVTVSSGGGGSGGGGSGGGGSAQSVVTQPPSMSGTPGQRVTISCSGSRSNIGRNYVYWYQQFPGTAPKLLIYRNNERPSGVPDRFSASKSGTSASLAISGLRSEDEADYYCATWDDSLSGTWVFGGGTKLTVLGAAA

SEQ ID NO: 6 - protein, 248 aa, Homo sapiens
LLESGGGLVQPGGSLRLSCAASGFTFSSYAMSWVRQAPGKGLEWVSAISGSGGSTYYADSVKGRFTISRDNSKNTLYLQMNSLRAEDTAVYYCARGGGRYDSSHGFDSWGRGTMVTVSSGGGGSGGGGSGGGGSALSYELTQPPSVSVAPGETATITCGGRSLGSKVVHWYQQKPGQAPTLVIYYDSVRPSGVPERFSASNSRLSATLTVSRVEAGDEADYYCQVWDRSSDHYVFGTGTKLTVLGAAA

SEQ ID NO: 7 - protein, 248 aa, Homo sapiens
QLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDWRWQQFGGWFDPWGRGTLVTVSSGGGGSGGGGSGGGGSALETTLTQSPATLSLSPGETATLFCRASQSVRNNLVWYQQKLGQAPRLLIFGASTRASGIPDRFTGSGSGTDFSLTITKLEPEDFAVYYCQRYGGFPITFGQGTRLEIKRAAA

SEQ ID NO: 8 - protein, 247 aa, Homo sapiens
QLVQSGGGLVQPGGSLRLACEASGFRFSSYGMSWVRQAPGKGLEWVSSMSDSGANTYYADSVKGRFTISRDNAKKMLYLQMSSLRGEDTAVYYCATLFRGSGYVRHWGRGTLVTVSSGGGGSGGGGSGGGGSAQAVLTQPSSASGTPGQRVIISCSGSSSNIASNYVYWYQQLPGTAPKLLISKNSRRPSGVPDRFSGSKSGTSASLAISELRSEDEADYYCAAWDDRLSGPAFGGGTKLTVLGAAA

SEQ ID NO: 9 - protein, 248 aa, Homo sapiens
KKPGSSVKVSCKASGGTFSSYAISWVRQAPGQGLEWMGGIIPMSGTPNYAQKFQDRVTITADKSTSTAYMELSSLRSEDTAVYYCARGGRYVDFGRGPSYHYYYMDVWGRGTLVTVSSGGGGSGGGGSGGGGSAQSVLTQPPSASGTPGQRVTISCSGATSNIGRNYVYWYHQLPGTAPKLLIYRNDQRPSGVPDRFSGSKSGTSASLAISGLRSDDEADYYCAAWDDNLSGLFFGGGTKLTVLGAAA

SEQ ID NO: 10 - protein, 247 aa, Homo sapiens
AQVQLQQWGPGLVKASEILSLNCTVSGSSLSSGGYYWSWIRQHPGKGLEWIGYIHYSGSTYYNPSLKSRVTISVDTSKNQFSLKLSSVTAADTAVYYCARVPLRFDGFDVWGQGTLVTVSSGGGGSGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKRAAA

SEQ ID NO: 11 - protein, 246 aa, Homo sapiens
ELKKPGSSVKVSCKAPRGTFNSYALNWVRQAPGQGLEWMGGIIPIFGSANYAPKFQGRVTITADESTTTAYLELSSLRSEDTAVYYCARALHLDYVWRTYNYYFDNWGKGTMVTVSSGGGGSGGGGSGGGGSALSSELTQDPAVSVALGQTVRITCQGDSLRSYYASWYQQKPGQAPVLVIYGKNSRPSGIPDRFSGSDSGNTASLTITGAQAEDEADYYCNSRDRSGNRVVFGGGTKLTVLGAAA

SEQ ID NO: 12 - protein, 248 aa, Homo sapiens
SLRLSCAASGFTFSSYAMSWVRQAPGKGLEWVSAISGSGGSTYYADSVKGRFTISRDNSKNTLYLQMNSLRAEDTAVYYCARGVTYHYDHDRRGVTAQIYNHGLDVWGRGTTVTVSSGGGGSGGGGSGGGGSAQAVLTQPSSASGTPGQRVTISCSGSSSNIGKNYVYWYQQLPGTAPKLLIYRNNQRPSGVPDRFSGSKSGTSASLAISGLRSEDEADYYCAARDNGLSAYVIFGGGTKLTVLGAAA

SEQ ID NO: 13 - protein, 247 aa, Homo sapiens
VKKPGESLKISCKGSGYSFPNYWIAWVRQMPGKGLEWMGIIYPGDSDTIYSPSFRGQVTISADKSISTAYLQWSSLKASDTAMYYCARQGCSGGKCYEKMYASDIWGRGTLVTVSSGGGGSGGGGSGGGGSALSYELTQPPSASGTPGQRVTISCSGSTSNIGRNSVFWHQQLPGTAPKVLISSDNQRPSGVSDRFSGSDSGTSASLVISRLRFEDEGDYYCAAWDDSLSAYVFGSGTKLTVLGAAA

SEQ ID NO: 14 - protein, 248 aa, Homo sapiens
AEVKKPGSSVRVSCKASGDTFSYNAINWVRQAPGQGLEWMGGIIPMFGTAKQAQKFQGRVTFTADESTSTAYMELTRLRSEDTAMYYCARRGSYSNYERGYYYHMDVWGQGTLVTVSSGGGGSGGGGSGGGGSALPVLTQPPSASGAPGQRITISCSGSTFNIGRNYVDWYKQLPGTAPKLFIYKNDQRPSGVPDRFSGSKSGTSASLVVSGLRSEDEADYYCLTWDDSLSGPVFGGGTKLTVLGAAA

SEQ ID NO: 15 - protein, 247 aa, Homo sapiens
LQESGPGLVKPSGTLSLTCAVSGGSINNNNWWSWVRQPPGKGLEWIGEIYQSGSTNYNPSLKSRVTISVDKSNNQFSLKMSSVTAADTAVYYCARLNWNHGPYYGMDVWGRGTLVTVSSGGGGSGGGGSGGGGSAQSVLTQPPSASGTPGQRVTISCSGSSSNIGSNFVYWYQQLPGTAPKLLIYRNNQRPSGVPDRFSASKSGTSASLAISGLRSEDEADYYCAAWDDRRVVFGGGTKLTVLGAAA

SEQ ID NO: 16 - protein, 245 aa, Homo sapiens
VQLQESGPGLVKPSETLSLTCTVSGGPVASSSYYWGFIRQPPGKGLEWIGSIYDGGYTYYSPSLKSRATISFDTSKNQVSLNLTSVTAADTAVYYCAKDPGSLSAFWGQGTLVTVSSGGGGSGGGGSGGGGSALDIQLTQSPSSLSASVGDRVTITCRTSQRISSYLNWYQQKPGKAPKLLIYAASSLQSGVPSRFSGSGSGTDFTLTISSLQPEDFATYYCQQSYSTPITFGQGTRLEIKRAAA

SEQ ID NO: 17 - protein, 248 aa, Homo sapiens
LLESGGGLVQPGGSLRLSCAASGFTFSSYAMSWVRQAPGKGLEWVSAISGSGGSTYYADSVKGRFTISRDNSKNTLYLQMNSLRAEDTAVYYCARDWRWQQFGGWFDPWGRGTLVTVSSGGGGSGGGGSGGGGSALDVVMTQSPATLSVSPGERVTLSCRASQSVGSKLAWYQQKPGQAPRLLIFGTSTRASGIPARFSGSGSGTEFTLTISSLQSEDFAVYYCQQYNNWPPYTFGQGTKVEIKRAAA

SEQ ID NO: 18 - protein, 247 aa, Homo sapiens
AEVKKPGDSVKVSCKASGYRFETYGFSWVRQAPGQGLEWMGWINTYNGKTNYAQKFQGRVTMTTDTSTSTAYMELRSLRSDDTAVYFCSRAEDDSRGYWNHYFSDYWGRGTTVTVSSGGGGSGGGGSGGGGSAQSVLTQPPSASGTPGQRVTISCSGSSSNIGSNYVYWYQQLPGTAPKLLIHKNNRRPSGVPDRFSGSKSGTSASLAISGLRSEDEADYHCAAWDDSLSAVVFGGGTKVTVLGAAA

SEQ ID NO: 19 - protein, 247 aa, Homo sapiens
LESGGGLVQPGGSLRLSCAASGFTFSSYAMSWVRQAPGKELEWVSAISGSGGSTYYADSVKGRFTISRDNSKNTLYLQMNSLRAEDTAVYYCARDWRWQQFGGWFDPWGRGTMVTVSSGGGGSGGGGSGGGGSALETTLTQSPGTLSLSPGDRATLSCRASHSVSHNHLAWYQQNPGQAPRLLIFGASSRAAGIPDRFSGSGSGTDFTLTISRLEPEDFASYYCQQYGSPRRTFGQGTKVEIKRAAA

SEQ ID NO: 20 - protein, 247 aa, Homo sapiens
KKPGSSVRVSCKAPGGTFGNSAISWVRQTPGQGLEWMGGIIPMFTTANYAQKFQGRVTITADKSTTTAHMELSSLRSEDTAVYYCARGGLGRFFDGPSHFSYYMEVWGKGTLVTVSSGGGGSGGGGSGGGGSAQSVLTQPPAASGTPGQRVTISCSGSNSNIGRNYVYWYQQLPGAAPKLLIYRNNQRPSGVPDRFSGSKSGPSASLAISGLRSEDEADYYCAAWDDSLSGPAFGGGTKLTVLGAAA

SEQ ID NO: 21 - protein, 246 aa, Homo sapiens
VQLVQSGAEVKKPGSSVKVSCKASGGTFSSDAISWVRQAPGQGLEWMGRIIPLINIPNYAQKFQGRVTITADKSTTTAYMELTSLRFEDAAVYYCARVNNWNAFDQWGRGTLVTVSSGGGGSGGGGSGGGGSALSSELTQDPAVSVALGQTVRITCQGDTLTSYYAAWYQQKPGQAPLLVFYGKDKRPSGIPERFSGSSSGNIASLTITGAQAEDEGDFYCSSRDSSGYRFVFGAGTKLTVLGAAA

SEQ ID NO: 22 - protein, 246 aa, Homo sapiens
KKPGSSVKVSCKASGGTFTSYAISWVRQAPGQGLEWMGGFIPVFGTANYAQKLQGRVTITADDSMTTVYMELSSLTSEDTGVYYCARDLMRLARRDEYYYYYMDVWGQGTMVTVSSGGGGSGGGGSGGGGSAQSVLTQPPAASGTYGQKITISCSGSSSNIGVNYVYWYRQFPGAAPHVVIYNNDQRPSGVPDRFSGSKSGTSASLAISGLRSEDEADYYCSTWDDTLSGYIFGVGTKVTVLGAAA

SEQ ID NO: 23 - protein, 239 aa, Homo sapiens
QPGGSLRLSCAASGFTFSSYAMSWVRQAPGKGLEWVSAISGSGGSTYYADSVKGRFTISRDNSKNTLYLQMNSLRAEDTAVYYCARDWRWQQFGGWFDPWGQGTLVTVSSGGGGSGGGGSGGGGSALSSELTQDPAVSVALGQTVRITCQGDNLRSFSASWYQLKPGQAPVLVIYGKNNRPSGIPDRFSASSSGNTASLAITGALAEDEADYYCNSRDSSGNPYVFGTGTKVTVLGAAA

SEQ ID NO: 24 - protein, 235 aa, Homo sapiens
SSVKVSCKISGGNLNRLTVTWVRQAPGQGLEWVGRILPDSVNQVVKFQRRLKLTSDTSTRTAYLELRSLKSEDTAVYYCAASSKIGFQVGELDYWGRGTLVTVSSGGGGSGGGGSGGGGSAQSVVTQPPSASATPGQRVTISCSGSSSNIGRNYVYWYQQVPGTAPQLLVYNNNQRPSGVPDRFSGSKSGTSASLGISGLRSEDEADYYCSTWDDSLSSPVFGGGTKLTVLGAAA

SEQ ID NO: 25 - protein, 238 aa, Homo sapiens
TFSSYAMSWVRQAPGKGLEWVSAISGSGGSTYYADSVKGRFTISRDNSKNTLYLQMNSLRAEDTAVYYCARGRRRERSINMIRGVRPQYDDSGMDVWGRGTLVTVSSGGGGSGGGGSGGGGSALSYVLTQPPSASGTPGHRVTISCSGSSSNIGSNYVYWYQQLPGTAPKLLIYRNNQRPSGVPDRFSGSKSGTSASLAISGLRSEDEADYYCAAWDDTLSGVLFGGGTKLTVLGAAA

SEQ ID NO: 26 - protein, 235 aa, Homo sapiens
YAMSWVRQAPGKGLEWVSAISGSGGSTYYADSVKGRFTISRDNSKNTLYLQMNSLRAEDTAVYYCARNTGKGITLVRGVYCQDCDRSSTSRMDVWGQGTLVTVSSGGGGSGGGGSGGGGSAQAVLTQPSSASGTPGQRVTISCSGSTSNIGRNYVDWYQQLPGTAPKLLIYRNNKRPSGVPDRFSGSKSGTSASLAISGLRSEDEADYYCAAWDDSLSGWVFGGGTKLTVLGAAA

SEQ ID NO: 27 - protein, 246 aa, Homo sapiens
GLVQPGGSPRLSCAASGFTFSSYAMSWVRQAPGKGLEWVSAISGSGGSTYYADSVKGRFTISRDNSKNTLYLQMNSLRAEDTAVYYCAKDMGYSYGYGTRGLFDYWGRGTMVTVSSGGGGSGGGGSGGGGSAQSVVTQPPSASGAPGQRITISCSGSTFNIGRNYVDWYKQLPGTAPKLFIYKNDQRPSGVPDRFSGSKSGTSASLVVSGLRSEDEADYYCLTWDDSLSGPVFGGGTKVTVLGAAA

SEQ ID NO: 28 - protein, 246 aa, Homo sapiens
ESGGGLVQPGGSLRLSCAASGFTFSSYAMSWVRQAPRKGLEWVSAISGSGGSTYYADSVKGRFTISRDNSKNTLYLQMNSLRAEDTAVYYCARDWRWQQFGGWFDPWGRGTTVTVSSGGGGSGGGGSGGGGSALETTLTQSPATLSVSPGDRATLSCRASQSIGGNLAWYQQKPGQPPRLLIFGASTRASGTPARFSGSGSGTEFTLTISSLQSEDFAVYYCQQYNNWPPWTFGQGTRLEIKRAAA

SEQ ID NO: 29 - protein, 244 aa, Homo sapiens
QPGGSLRLSCAASGFTFSSYAMSWVRQAPGKGLEWVSAISGSGGSTYYADSVKGRFTISRDNSKNTLYLQMNSLRAEDTAVYYCAKGDGVVAGTTYYYYGMDVWGRGTTVTVSSGGGGSGGGGSGGGGSAQSVLTQPPSASGAPGQRITISCSGSTFNIGRNYVDWYKQLPGTAPKLFIYKNDQRPSGVPDRFSGSKSGTSASLVVSGLRSEDEADYYCLTWDDSLSGPVFGGGTKLTVLGAAA

SEQ ID NO: 30 - protein, 233 aa, Homo sapiens
ASGFGLNGYEMHWVRQAPGQRLEWLGRINAAIGDTRYSREFQDRVSITRDMSANTVYMEMSRLRFEDTAVYYCVRFHDWRHCNSATCQPPFDHWGKGTLVTVSSGGGGSGGGGSGGGGSALSSELTQDPAVSVALGQTVRITCQGDSLRYYSASWYRQKPGQAPVIVMYGNTRRPSGIPDRISGSSSGNTASLTISGAQAEDEADYYCNSRDSSGNHLVFGGGTKLTVLGAAA

SEQ ID NO: 31 - protein, 246 aa, Homo sapiens
VQPGGSLRLSCAASGFTFSSYAMSWVRQAPGKGLEWVSAISGSGGSTYYADSVKGRFTISRDNSKNTLYLQMNSLRAEDTAVYYCARDHRSGRGGGSYLLRPLDYWGQGTMVTVSSGGGGSGGGGSGGGGSALPVLTQPPSASGTPGQRVTISCSGSSSNIGRNYVYWYQQLPGTAPKLLIYRNNLRPSGVPDRFSGSKSGTSASLAISGLRSEDEADYYCAAWDDTLSGVVFGGGTKLTVLGAAA

SEQ ID NO: 32 - protein, 244 aa, Homo sapiens
EVRKPGASVKISCKASGFTFTSYLFHWVRQAPGQRLEWMGWINAGNGNTKYSPKFQGRVTLTGDTSTSTTYMELSSLTSEDTAVYYCARDQVFYESGSYYIRPSFDFWGRGTLVTVSSGGGGSGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKRAAA

SEQ ID NO: 33 - protein, 235 aa, Homo sapiens
VRPGGSLRLSCAASGFTFDDYGMSWVRQAPGKGLEWVSGINWNGGSTGYADSVKGRFTISRDNAKNSLYLQINSLRAEDTAVYYCARRRYALDYWGRGTMVTVSSGGGGSGGGGSGGGGSALSSELTQDPATVSVALGQTVRITCQGDSLDKYYATWYQQKPGQAPLLVFFSENRRPSGIPDRFSGSNSGNTASLTITGAQAEDEADYYCNSREIGTNRILFGGGTKLTVLGAAA

SEQ ID NO: 34 - protein, 245 aa, Homo sapiens
LVQPGGSLRLSCAAAGFTFSTFEMNWVRQAPGKGLEWVSYISGSGHAIYYADSVKGRFTISRDNANNSLYLQMNSLTAEDTAVYYCAREKYQLLLGKYDYGMDVWGRGTTVTVSSGGGGSGGGGSGGGGSALPVLTQPPSASGTPGQRVTISCSGSSSNIGSNTLNWYQQLPGTAPKLLIYSNDQRPSGVPDRFSGSKSGTSASLAISGLQSEDEADYYCAAWDDSLNGWVFGGGTKVTVLGAAA

SEQ ID NO: 35 - protein, 233 aa, Homo sapiens
RASGGTSSSSAFSWVRQAPGQGLQWMGGIIPLFGAANYAQKVRAGLTITADESTGTSYMKLENLQSDDTAVYFCATNGQTRSPPGYYYGMDVWGRGTLVTVSSGGGGSGGGGSGGGGSAQSVLTQLPSASGAPGQRITISCSGSTFNIGRNYVDWYKQLPGTAPKLFIYKNDQRPSGVPGRFSGSKSGTSASLVVSGLRSEDEADYYCLTWDDSLSGPVFGGGTKLTVLGAAA

SEQ ID NO: 36 - protein, 247 aa, Homo sapiens
ACKGFGYTFVDHGISWVRQAPGQGLEWMGWINTHDGHTNYAQKTQARLTMTTDASINTSYMELRSLTSDDTAVYYCARGGETRTAHRSRRATNDNGYPYYSSGLDVWGQGTLVTVSSGGGGSGGGGSGGGGSAQAVLTQPSSASGTPGQRVTISCSGSSSNIGSNYVYWYQQLPGTAPKLLIYRNNQRPSGVPDRFSGSKSGTSASLAISGLRSEDEADYYCAAWDDSLSGWVFGGGTKLTVLGAAA

SEQ ID NO: 37 - protein, 245 aa, Homo sapiens
PGASVKVSCKASGYTFTSYYMHWVRQAPGQGLEWMGIINPSGGSTSYAQKFQGRVTMTRDTSTSTVYMELSSLRSEDTAVYYCARGSGARMVRGVIIDPYGMDVWGRGTLVTVSSGGGGSGGGGSGGGGSAQSVLTQPPSASGTPGQRVTISCSGSSSNVGSNYVSWYQQFPGTAPKLLIYRNNQRPSGVPDRFSGSKSGISASLAISGLRSEDEADFYCVAWDDSLREYVFGTGTKVTVLGAAA

SEQ ID NO: 38 - protein, 247 aa, Homo sapiens
ESGGGLVQPGGSLRLSCAASGFTFSSYAMSWVRQAPGKGLEWVSAISGSGGSTYYADSVKGRFTISRDNSKNTLYLQMNSLRAEDTAVYYCAKGGTRVTHRGGFDIWGRGTMVTVSSGGGGSGGGGSGGGGSALPVLTQPPSASGAPGQRITISCSGSTFNIGRNYVDWYKQLPGTAPKLFIYKNDQRPSGVPDRFSGSKSGTSASLVVSGLRSEDEADYYCLTWDDSLSGPVFGGGTKLTVLGAAA

SEQ ID NO: 39 - DNA, 739 bp, Homo sapiens
gggcctcagtgaaggtctcctgcaagacctctggatacaccttcatcgcctattatattcattgggtgcgacaggcccctggacaagggcttgagtggatgggacggatcaaccctaacactggtggcataaaccttgcacagaagtttcagggcagggtcaccgtgaccagggacacgtccatcagcacagcccacatggagctgagtaggctgagctctgacgacacggccgtatactactgtgcgagagagaggatcgtaccagctggtctgaggaaccgtgggatggtgactgcggttggaatggacgtctggggccggggaaccctggtcaccgtctcgagtggaggcggcggttcaggcggaggtggctctggcggtggcggaagtgcacagtctgtcgtgacgcagccgccctcaatgtctgggacccccgggcagagggtcaccatctcttgttctgggagcaggtccaacattggaaggaattatgtatactggtaccagcagttcccaggaacggcccccaaactcctcatttataggaataatgaacggccctcaggggtccctgaccgattctctgcctccaagtctggcacctcagcctccctggccatcagtggactccggtccgaggatgaggctgattattactgcgcaacgtgggatgacagtctgagtgggacttgggtgttcggcggagggaccaagctgaccgtcctaggtgcggccgc

SEQ ID NO: 40 - DNA, 743 bp, Homo sapiens
ctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggtggggggcgatatgatagtagtcacggctttgactcctggggccgggggacaatggtcaccgtctcgagtggaggcggcggttcaggcggaggtggctctggcggtggcggaagtgcactttcctatgagctgactcagccaccctcagtgtcggtggccccaggagagacggccacaattacctgtgggggacgcagccttggctccaaagttgtgcattggtatcagcagaagccaggccaggcccctacattggtcatttattatgattccgtccggccctcgggggtccctgagcgattctctgcctccaactctcggttgtcggccaccctgaccgtcagcagggtcgaagccggggatgaggccgactattattgtcaggtgtgggatagaagtagtgaccattatgtcttcggaactgggaccaagctgaccgtcctaggtgcggccgc

SEQ ID NO: 41 - DNA, 743 bp, Homo sapiens
cagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagagattggagatggcaacaatttgggggctggttcgacccctggggccgaggaaccctggtcaccgtctcgagtggaggcggcggttcaggcggaggtggctctggcggtggcggaagtgcacttgaaacgacactcacgcagtctcctgccaccctgtctctgtctccaggggaaacagccaccctcttctgcagggccagtcagagtgttaggaacaacttagtctggtaccagcagaagcttggccaggctcccagactcctcatctttggtgcatccaccagggccagtggcatcccagacaggttcactggcagtgggtctgggacagacttcagtctcaccatcaccaaactggagcctgaggattttgcagtgtattactgtcagcggtatggtggtttcccgatcaccttcggccaagggacacgactggagattaaacgtgcggccgc

SEQ ID NO: 42 - DNA, 740 bp, Homo sapiens
cagctggtgcagtctgggggaggcttggtgcagccgggggggtccctgagactcgcctgtgaagcctctggattcaggtttagcagctatggcatgagctgggtccgccaggctccagggaaggggcttgagtgggtctcttccatgagcgatagtggtgccaatacatactacgcagactccgtgaagggccgattcaccatctccagagacaatgccaagaagatgttgtatctacaaatgagcagcctgagaggcgaggacacggccgtgtattactgtgcgactctctttaggggaagtggctacgttcgtcactggggcaggggaaccctggtcaccgtctcgagtggaggcggcggttcaggcggaggtggctctggcggtggcggaagtgcacaggctgtgctgactcagccgtcctcagcgtctgggacccccgggcagagggtcatcatctcttgttctggaagcagctccaacatcgcaagtaattatgtatactggtaccagcaactcccaggaacggcccccaaactcctcatctctaagaatagtcggcggccctcaggggtccctgaccgattctctggctccaagtctggcacctcagcctccctggccatcagtgaactccggtccgaggatgaggctgattattactgtgcagcatgggatgacaggctgagtggtccggcattcggcggagggaccaagctgaccgtcctaggtgcggccgc

SEQ ID NO: 43 - DNA, 743 bp, Homo sapiens
aagaagcctgggtcctcggtgaaggtctcctgcaaggcttctggaggcaccttcagcagttatgctattagttgggtgcgacaggcccctggacaagggcttgagtggatgggagggatcattcctatgtctggtacaccaaactacgcacagaagttccaggacagagtcacgattaccgcggacaaatccacgagcacagcctacatggagctgagcagcctgagatctgaggacacggccgtgtattactgtgcgaggggggggcgctacgttgacttcggtcgtggcccttcgtaccactactactacatggacgtctggggcaggggaaccctggtcaccgtctcgagtggaggcggcggttcaggcggaggtggctctggcggtggcggaagtgcacagtctgtgttgacgcagccgccctcagcgtctgggacccccggacagagggtcaccatctcttgttctggggccacctccaacatcggaaggaattatgtttactggtaccaccaactcccagggacggcccccaagctcctcatctataggaatgatcagcgtccctcaggggtccctgaccgattctctgggtccaagtctggcacctcagcctccctggccatcagtggcctccggtccgacgatgaggctgattattactgtgctgcgtgggacgacaacctgagtggtctatttttcggcggagggaccaagctgaccgtcctaggtgcggccgc

SEQ ID NO: 44 - DNA, 740 bp, Homo sapiens
gcccaggtgcagctacagcagtggggcccaggactggtgaaggcttcggagatcctgtccctcaactgcactgtctctggtagctccctcagcagtggtggttactactggagctggatccgccagcacccagggaagggcctggagtggattgggtacatccattacagtgggagcacgtactacaacccgtccctcaagagtcgagttaccatatcagtagacacgtctaagaaccagttctccctgaagctgagctctgtgactgccgcggacacggctgtgtattattgtgcgagagttccgttgagatttgatggttttgatgtctggggccaaggcaccctggtcaccgtctcgagtggtggaggcggttcaggcggaggtggcagcggcggtggcggatcggacatccagatgacccagtctccttccaccctgtctgcatctattggagacagagtcaccatcacctgccgggccagtgagggtatttatcactggttggcctggtatcagcagaagccagggaaagcccctaaactcctgatctataaggcctctagtttagccagtggggccccatcaaggttcagcggcagtggatctgggacagatttcactctcaccatcagcagcctgcagcctgatgattttgcaacttattactgccaacaatatagtaattatccgctcactttcggcggagggaccaagctggagatcaaacgtgcggccgc

SEQ ID NO: 45 - DNA, 739 bp, Homo sapiens
ctgagctgaagaagcctgggtcctcggtaaaggtctcctgcaaggctcctagaggcaccttcaacagttatgctctcaactgggtgcgacaggcccctggacaagggcttgagtggatgggagggatcatccctatttttggtagtgcaaattacgcaccgaagttccagggcagagtcaccattaccgcggacgaatccacgaccacagcctacttggagctgagcagcctgagatctgaggacacggccgtatattactgtgcgcgagctctccatttggattacgtttggaggacttataattactactttgacaactggggcaaagggacaatggtcaccgtctcgagtggaggcggcggttcaggcggaggtggctctggcggtggcggaagtgcactttcttctgagctgactcaggaccctgctgtgtctgtggccttgggacagacagtcaggatcacatgccagggagacagcctcagaagttattatgcaagctggtaccagcagaagccaggacaggcccctgtccttgtcatctatggtaaaaatagtcggccctcagggatcccagaccgattctctggctccgactcaggaaacacagcttccttgaccatcactggggctcaggcggaagatgaggctgactattactgtaactcccgggacagaagtggtaaccgcgtggtcttcggcggagggaccaagctgaccgtcctaggtgcggccgc

SEQ ID NO: 46 - DNA, 743 bp, Homo sapiens
tccctgagactctcctgtgcggcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggggttacgtatcactatgaccatgacaggcgtggtgtgaccgcgcaaatatataaccacggtttggacgtctgggggagggggaccacggtcaccgtctcgagtggaggcggcggttcaggcggaggtggctctggcggtggcggaagtgcacaggctgtgctgactcagccgtcctcagcgtctgggacccccgggcagagggtcaccatctcttgttctggaagcagctccaacatcggaaagaattatgtatactggtatcagcagctcccaggaacggcccccaaactcctcatctataggaataatcagcggccctcaggagtccctgaccgattctctggctccaagtctggcacctcagcctccctggccatcagtgggctccggtccgaggatgaggctgattattattgtgcggcacgggataacggcctgagtgcttatgtgatattcggcggagggaccaagctgaccgtcctaggtgcggccgc

SEQ ID NO: 47 - DNA, 742 bp, Homo sapiens
aggtgaaaaagcccggggagtctctgaagatctcctgcaagggttctggatacagctttcccaactactggatcgcctgggtgcgccagatgcccgggaaaggcctggagtggatggggatcatctatcctggtgactctgatactatatacagcccgtccttccgaggccaggtcaccatctcagccgacaagtccatcagcaccgcctacctgcagtggagcagcctgaaggcctcggacaccgccatgtattactgtgcgagacagggttgtagtggtggtaaatgctacgagaaaatgtatgcttctgatatctggggcaggggaaccctggtcaccgtctcgagtggaggcggcggttcaggcggaggtggctctggcggtggcggaagtgcactttcctatgagctgactcagccaccctcagcgtctgggacccccgggcagagggtcaccatctcttgttctggaagcacgtccaacatcggaaggaattctgtattttggcaccagcagttaccaggaacggcccccaaagtcctcatctcttctgataatcagcgaccctcaggggtctctgacagattctctggctccgactctggcacctcagcctccctggtcatcagtcgcctccggttcgaagatgagggtgattactactgtgcagcatgggatgacagtctgagtgcttatgtcttcggaagtgggaccaagctgaccgtcctaggtgcggccgc

SEQ ID NO: 48 - DNA, 745 bp, Homo sapiens
gggctgaggtgaagaagcctgggtcctcggtgagggtctcctgcaaggcttctggagacaccttcagctacaatgctatcaactgggtgcgacaggcccctggacaagggcttgagtggatgggagggatcatccctatgtttggtacagcaaagcaggcacagaagttccagggcagagtcacgtttaccgcggacgaatccacgagcacagcctacatggagttgactaggctgagatccgaggacacggccatgtattactgtgcgcgacggggctcgtacagtaattacgagagggggtattactatcacatggacgtctggggccagggaaccctggtcaccgtctcgagtggaggcggcggttcaggcggaggtggctctggcggtggcggaagtgcactgcctgtgctgactcagccaccctcagcgtctggggcccccgggcagaggatcaccatctcttgttccggaagcaccttcaacatcgggagaaattatgttgactggtataaacaactccccggaacggcccctaaactcttcatctataagaatgatcagcgaccctcaggggtccctgaccgattctctggctccaagtctggcacctcagcctccctggtcgtaagtggactccgctccgaggatgaggctgattattactgtctgacttgggatgacagcctgagtggtccggtgttcggcggggggaccaagctgaccgtcctaggtgcggccgc

SEQ ID NO: 49 - DNA, 741 bp, Homo sapiens
gctgcaggagtccggcccaggactggtgaagccttcggggaccctgtccctcacctgcgctgtctctggtggctccatcaacaataataattggtggagttgggtccgccagcccccagggaaggggctggagtggattggggaaatctatcagagtgggagcaccaactacaacccgtccctcaagagtcgagtcaccatatcagtagacaagtccaacaaccagttctccctgaagatgagttctgtgaccgccgcggacacggccgtgtattactgtgcgaggcttaactggaaccacgggccctactacggtatggacgtctggggcaggggcaccctggtcaccgtctcgagtggaggcggcggttcaggcggaggtggctctggcggtggcggaagtgcacagtctgtgctgacgcagccgccctcagcgtctgggacccccggacagagagtcaccatctcttgttctggaagcagctccaacatcggaagtaattttgtatactggtaccagcagctcccaggaacggcccccaaactcctcatctataggaataatcagcggccctcaggggtccctgaccgattctctgcctccaagtctggcacctcagcctccctggccatcagtgggctccggtccgaggatgaggctgattattactgtgcggcatgggatgacaggcgtgtggtattcggcggagggaccaagctgaccgtcctaggtgcggccgc

SEQ ID NO: 50 - DNA, 735 bp, Homo sapiens
ggtgcagctgcaggagtcgggcccaggactggtgaagccttcggagaccctgtccctcacctgcactgtctctggcggccccgtcgccagtagtagttactactggggcttcatccgccagcccccaggaaaagggctggagtggattgggagtatttatgatggtggctacacctactacagcccgtccctaaagagtcgagctaccatatccttcgacacgtccaagaaccaggtctccctgaacctgacctctgttaccgccgcggacacggccgtctattactgtgcgaaagacccgggcagtttgagcgccttctggggccagggaaccctggtcaccgtctcgagtggaggcggcggttcaggcggaggtggctctggcggtggcggaagtgcacttgacatccagttgacccagtctccatcctccctgtctgcgtctgtaggagacagagtcaccatcacttgccggacaagtcagcgcattagcagctatttaaattggtatcagcagaagccagggaaagcccctaagctcctgatctatgctgcatccagtttgcaaagtggggtcccatcaaggttcagtggcagtggttctgggacagatttcactctcaccatcagcagtctgcaacctgaagattttgcaacttactactgtcaacagagttacagtaccccgatcaccttcggccaagggacacgactggagattaaacgtgcggccgc

SEQ ID NO: 51 - DNA, 743 bp, Homo sapiens
ctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagagattggagatggcaacaatttgggggctggttcgacccctggggcagaggcaccctggtcaccgtctcgagtggaggcggcggttcaggcggaggtggctctggcggtggcggaagtgcacttgatgttgtgatgactcagtctccagccaccctgtctgtgtctccaggggaaagagtcaccctctcctgcagggccagtcagagtgttggcagcaagttggcctggtaccagcagaaacctgggcaggctcccaggctcctcatctttggtacatcgaccagggccagtggtatcccagccaggttcagtggcagtgggtctgggacagagttcactctcaccatcagcagcctgcagtctgaagattttgcagtttattactgtcagcagtataataactggcctccgtacacttttggccaggggaccaaggtggaaatcaaacgtgcggccgc

SEQ ID NO: 52 - DNA, 740 bp, Homo sapiens
gctgaggtgaagaagcctggggactcagtgaaggtctcctgcaaggcctctggttacaggtttgaaacctatggtttcagctgggtgcgacaggcccctggacaagggcttgagtggatgggatggatcaacacttacaatggtaagacaaattatgcacagaagttccagggcagagtcaccatgaccacagacacgtccacgagcacagcctacatggagttgaggagcctgagatcggacgacacggccgtgtatttttgttcgagagctgaggatgatagcagaggttattggaaccattacttctccgactactgggggagggggaccacggtcaccgtctcgagtggaggcggcggttcaggcggaggtggctctggcggtggcggaagtgcacagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatccataagaataatcggcggccctcaggggtccctgaccgattctctggctccaagtctggcacctcagcctccctggccatcagtgggctccggtccgaggatgaggctgattatcactgtgcagcgtgggatgacagcctgagtgctgtggttttcggcggagggaccaaggtcaccgtcctaggtgcggccgc

SEQ ID NO: 53 - DNA, 740 bp, Homo sapiens
ttggagtctgggggaggcttggtacagcctggggggtccctgagactctcctgtgcagcctctggattcacctttagcagctatgccatgagctgggtccgccaggctccagggaaggagctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagagattggagatggcaacaatttgggggctggttcgacccctggggccgagggacaatggtcaccgtctcgagtggaggcggcggttcaggcggaggtggctctggcggtggcggaagtgcacttgaaacgacactcacgcagtctccaggcaccctgtctttgtctccaggtgatagagccaccctctcctgcagggccagtcacagtgttagtcacaaccacttagcctggtaccagcaaaatcctggccaggctcccaggctcctcatttttggtgcatccagcagggccgctggcatccctgacaggttcagtggcagtgggtctgggacagacttcactctcaccatcagcagactggagcctgaagattttgcatcatattactgtcagcagtatggtagcccccggcggacgttcggccaagggaccaaggtggaaatcaaacgtgcggccgc

SEQ ID NO: 54 - DNA, 741 bp, Homo sapiens
gaagaagcctgggtcctcggtgagggtctcctgcaaggctcctggaggcaccttcggcaactctgctatcagctgggtgcgacagacccctggacaagggcttgagtggatgggaggaatcattcctatgtttactacagcaaactacgcacagaagttccagggcagagtcaccattaccgcggacaaatccacgaccacagcccacatggagctgagcagcctgagatctgaggacacggccgtctattactgtgcgagaggcggactgggacgattttttgacggcccctcccacttctcctactacatggaagtctggggcaaaggaaccctggtcaccgtctcgagtggaggcggcggttcaggcggaggtggctctggcggtggcggaagtgcacagtctgtgctgacgcagccgcccgcagcgtctgggacccccgggcagagggtcaccatctcttgttctggaagcaactccaacatcggaagaaattatgtctactggtatcagcagctcccaggagcggcccccaaactcctcatctataggaataatcagcggccctcaggggtccctgaccgattctctggctccaagtccggcccctcagcctccctggccatcagtgggctccggtccgaggatgaggctgattattactgtgcagcatgggatgacagcctgagtggccctgcattcggcggagggaccaagctgaccgtcctaggtgcggccgc

SEQ ID NO: 55 - DNA, 739 bp, Homo sapiens
aggtccagctggtacagtctggggctgaggtgaagaagcctgggtcgtcggtgaaggtctcctgcaaggcttctggaggcaccttcagcagcgatgctatcagctgggtgcgacaggcccctggacaaggacttgagtggatgggaaggatcatccctctaattaatataccaaactacgcacagaagttccagggcagagtcacgattaccgcggacaaatccacgaccacagcctacatggagctgaccagcctaagatttgaggacgcggccgtgtattactgtgcgagagtgaataactggaacgcctttgaccagtggggccggggaaccctggtcaccgtctcgagtggaggcggcggttcaggcggaggtggctctggcggtggcggaagtgcactttcttctgagctgactcaggaccctgctgtgtctgtggccttgggacagacagtcaggattacatgccaaggagacaccctcacaagttattatgcggcctggtaccagcagaagccaggacaggcccccctccttgtcttctatggtaaagacaagcggccctcagggatcccagagcgattctctggctccagctcaggaaatattgcttccttgaccatcactggggctcaggcggaggatgagggtgacttttactgtagttcccgggacagcagtgggtaccgttttgtcttcggggctgggaccaagctgaccgtcctaggtgcggccgc

SEQ ID NO: 56 - DNA, 738 bp, Homo sapiens
gaagaagcctgggtcctcggtgaaggtctcctgcaaggcttctggaggcaccttcaccagctatgcaatcagttgggtgcgacaggcccctggacaagggcttgagtggatgggagggttcatccctgtatttggcacagcaaactacgcacagaagttgcagggcagagtcacgatcaccgcggacgattccatgaccacagtgtacatggagctgagtagcctgacctctgaagacacgggcgtgtattactgtgcgagagatctcatgcggctggcccgtcgcgatgaatactactattactacatggacgtctggggccaagggacaatggtcaccgtctcgagtggaggcggcggttcaggcggaggtggctctggcggtggcggaagtgcacagtctgtgctgactcagccacccgcagcgtctgggacctacgggcagaagatcaccatctcttgttctggaagcagttccaatatcggagttaattatgtttactggtaccggcaattcccaggagcggccccccacgtcgtcatctataataatgatcagcggccctcaggggtccctgaccgattctctggctccaagtctggcacctccgcctccctggccatcagtgggctccggtccgaggatgaggctgattattattgttccacatgggatgacaccctgagtggttatatcttcggagttgggaccaaggtcaccgtcctaggtgcggccgc

SEQ ID NO: 57 - DNA, 716 bp, Homo sapiens
cagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagagattggagatggcaacaatttgggggctggttcgacccctggggccagggcaccctggtcaccgtctcgagtggaggcggcggttcaggcggaggtggctctggcggtggcggaagtgcactttcttctgagctgactcaggaccctgctgtgtctgtggccttgggacagacagtcaggatcacatgccaaggagacaacctcagaagtttttctgcaagctggtaccagctgaagccaggacaggcccctgtacttgtcatctatggtaagaacaaccggccctcagggatcccagaccgattctctgcctccagctcaggaaacacagcttccttggccatcactggggctctggcggaagatgaggctgactactactgtaactcccgggacagcagtggtaacccttatgtcttcggaactgggaccaaggtcaccgtcctaggtgcggccgc

SEQ ID NO: 58 - DNA, 706 bp, Homo sapiens
ggtcttcggtgaaggtctcctgcaaaatttccggaggcaatctcaataggcttactgtcacctgggtgcgacaggcccctggacaaggccttgagtgggtgggcaggattcttcccgactcagtaaatcaagtcgtgaagttccagcgcagactcaaactgacctctgacacttccacgcgcacagcctatttagaactgaggagcctgaaatctgaagacacggccgtctattattgtgcggcgtcatctaaaataggcttccaggttggggagctcgactactggggccggggcaccctggtcaccgtctcgagtggaggcggcggttcaggcggaggtggctctggcggtggcggaagtgcacagtctgtcgtgacgcagccgccctcagcgtctgctacccccgggcagagggtcaccatctcttgttctggaagcagctccaacatcggaagaaattatgtctactggtaccagcaggtcccaggaacggccccccaactcctcgtctataacaataatcagcggccctcaggggtccctgaccgattctctggctccaagtctggcacctcagcctccctgggcatcagtgggctccggtccgaggatgaggctgattattactgttcaacatgggatgacagcctgagtagtccggtattcggcggggggaccaagctgaccgtcctaggtgcggccgc

SEQ ID NO: 59 - DNA, 714 bp, Homo sapiens
cacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggtagacggcgggagaggagtattaatatgattcggggagttagaccacaatacgacgactctggcatggacgtctggggccggggcaccctggtcaccgtctcgagtggaggcggcggttcaggcggaggtggctctggcggtggcggaagtgcactttcctatgtgctgactcagccaccctcagcgtctgggacccccgggcatagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacaccctaagtggtgtcctattcggcggagggaccaagctgaccgtcctaggtgcggccgc

SEQ ID NO: 60 - DNA, 706 bp, Homo sapiens
gctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaaatacaggaaagggcattactttggttcggggagtatattgtcaggactgtgaccgcagttctacatcccgcatggacgtctggggccagggcaccctggtcaccgtctcgagtggaggcggcggttcaggcggaggtggctctggcggtggcggaagtgcacaggctgtgctgactcagccgtcctcagcgtctgggacccccgggcagagggtcaccatctcttgttctggaagcacctccaacatcggaaggaattatgtagattggtaccagcagctcccaggaacggcccccaaactcctcatctataggaataataagcggccctcaggggtccctgaccgattctctggctccaagtctggcacctcagcctccctggccatcagtgggctccggtccgaggatgaggctgattattactgtgcagcttgggatgacagcctgagtggttgggtattcggcggagggaccaagctgaccgtcctaggtgcggccgc

SEQ ID NO: 61 - DNA, 738 bp, Homo sapiens
aggcttggtacagcctggggggtccccgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcaaaagatatgggatacagttatggttacgggacgaggggcctctttgactactggggccgagggacaatggtcaccgtctcgagtggaggcggcggttcaggcggaggtggctctggcggtggcggaagtgcacagtctgtcgtgacgcagccgccctcagcgtctggggcccccgggcagaggatcaccatctcttgttccggaagcaccttcaacatcgggagaaattatgttgactggtataaacaactccccggaacggcccccaaactcttcatctataagaatgatcagcgaccctcaggggtccctgaccgattctctggctccaagtctggcacctcagcctccctggtcgtaagtggactccgctccgaggatgaggctgattattactgtctgacttgggatgacagcctgagtggtccggtgttcggcggggggaccaaggtcaccgtcctaggtgcggccgc

SEQ ID NO: 62 - DNA, 739 bp, Homo sapiens
tggagtctgggggaggcttggtacagcctggggggtccctgagactctcctgtgcagcctctggattcacctttagcagctatgccatgagctgggtccgccaggctccacggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagagattggagatggcaacaatttgggggctggttcgacccctgggggcgagggaccacggtcaccgtctcgagtggaggcggcggttcaggcggaggtggctctggcggtggcggaagtgcacttgaaacgacactcacgcagtctccagccaccctgtctgtctctccgggggacagagccaccctctcctgcagggccagtcaaagtattggtggcaacttagcctggtaccagcagaaacctggccagcctcccaggctcctcatctttggtgcatccactagggcctctggtaccccagccaggttcagtggcagtgggtctgggacagagttcactctcaccatcagcagcctgcagtctgaagattttgcagtttattactgtcagcagtataataactggcctccatggactttcggccaagggacacgactggagattaaacgtgcggccgc

SEQ ID NO: 63 - DNA, 732 bp, Homo sapiens
acagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgaaaggggacggggtagtggctggaactacgtactactactacggtatggacgtctgggggcgagggaccacggtcaccgtctcgagtggaggcggcggttcaggcggaggtggctctggcggtggcggaagtgcacagtctgtgctgacgcagccgccctcagcgtctggggcccccgggcagaggatcaccatctcttgttccggaagcaccttcaacatcgggagaaattatgttgactggtataaacaactccccggaacggcccccaaactcttcatctataagaatgatcagcgaccctcaggggtccctgaccgattctctggctccaagtctggcacctcagcctccctggtcgtaagtggactccgctccgaggatgaggctgattattactgtctgacttgggatgacagcctgagtggtccggtgttcggcggggggaccaagctgaccgtcctaggtgcggccgc

SEQ ID NO: 64 - DNA, 699 bp, Homo sapiens
ggcctctggattcggcctcaatggctatgaaatgcattgggtgcgccaggcccccggacaaaggcttgagtggctgggccggatcaacgctgccattggcgacacacggtattcaagggagttccaggatagagtctccattaccagagacatgtccgcgaacacagtctacatggagatgagcaggctgagatttgaagacacggctgtttattattgtgtgagattccacgattggcgacattgtaatagtgccacctgtcagcccccttttgaccactggggcaagggaaccctggtcaccgtctcgagtggaggcggcggttcaggcggaggtggctctggcggtggcggaagtgcactttcttctgagctgactcaggaccctgctgtgtctgtggccttgggacagacagtcaggatcacatgccaaggagacagcctcagatactattctgcaagttggtaccggcagaagccagggcaggcccctgttattgtcatgtatggtaacacccgccggccctcagggatcccagaccgaatctctggctccagctcaggaaacacagcttccttgaccatcagtggggctcaggcggaagatgaggctgactattattgtaactcccgagacagtagtggtaaccatctggtcttcggcggagggaccaagctgaccgtcctaggtgcggccgc

SEQ ID NO: 65 - DNA, 737 bp, Homo sapiens
gtacagcctggggggtccctgagactctcctgtgcagcctctggattcacctttagcagctatgccatgagctgggtccgccaggctccagggaaggggctggagtgggtctcagctattagtggtagtggtggcagcacatactacgcagactccgtgaagggccggttcaccatctccagagacaattccaagaacacgctgtatctgcaaatgaacagcctgagagccgaggacacggccgtgtattactgtgcgagagatcatcggtcgggacgcggaggtgggagctacttactacgccctttggactactggggccaagggacaatggtcaccgtctcgagtggaggcggcggttcaggcggaggtggctctggcggtggcggaagtgcactgcctgtgctgactcagccaccctcagcgtctgggacccccgggcagagggtcaccatctcttgttctggaagcagctccaacatcggaaggaattatgtatactggtaccagcagctcccaggaacggcccccaaactactcatctatagaaataatctgcggccctcaggggtccctgaccgattctctggctccaagtctggcacctcagcctccctggccatcagtgggctccggtccgaggatgaggctgattattactgtgcagcatgggatgacaccctgagtggtgtggtattcggcggagggacaaagctgaccgtcctaggtgcggccgc

SEQ ID NO: 66 - DNA, 733 bp, Homo sapiens
cggaggtgaggaagcctggggcctcagtgaagatttcctgcaaggcttctggattcacgttcactagttatctattccattgggtgcgccaggcccccggacaaaggcttgagtggatggggtggatcaacgctggcaatggaaacacaaaatattcaccgaagttccagggcagagttacccttaccggggacacatccacgagcacaacctacatggagctgagcagcctgacatctgaggacacggctgtttattactgtgcgagagatcaggtgttctatgagagtggttcttactacatacgcccttcttttgacttctggggcaggggcaccctggtcaccgtctcttcaggtggaggcggttcaggcggaggtggcagcggcggtggcggatcggacatccagatgacccagtctccttccaccctgtctgcatctattggagacagagtcaccatcacctgccgggccagtgagggtatttatcactggttggcctggtatcagcagaagccagggaaagcccctaaactcctgatctataaggcctctagtttagccagtggggccccatcaaggttcagcggcagtggatctgggacagatttcactctcaccatcagcagcctgcagcctgatgattttgcaacttattactgccaacaatatagtaattatccgctcactttcggcggagggaccaagctggagatcaaacgtgcggccgc

SEQ ID NO: 67 - DNA, 706 bp, Homo sapiens
tggtacggcctggggggtccctgagactctcctgtgcagcctctggattcacctttgatgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaataaacagtctgagagccgaggacacagccgtgtattactgtgcaagaaggcggtatgcgttggattattggggcagagggacaatggtcaccgtctcgagtggaggcggcggttcaggcggaggtggctctggcggtggcggaagtgcactttcttctgagctgactcaggaccctgctactgtgtctgtggccttgggacagacagtcaggataacttgtcagggcgacagcctcgacaaatattatgcaacctggtatcaacagaagcctggacaggcccctctacttgtcttcttttctgaaaacaggcggccctcagggatcccagaccgtttctctggctccaactcgggaaacacagcttccttgaccatcactggggctcaggcggaggatgaggctgactattactgcaactcccgggaaatcggtactaatcgaatcctattcggcggagggaccaagctgaccgtcctaggtgcggccgc

SEQ ID NO: 68 - DNA, 734 bp, Homo sapiens
ttggttcagcctggagggtccctgagactctcctgtgcagccgctggattcaccttcagtacttttgaaatgaattgggtccgccaggccccagggaaggggctggagtgggtttcatatattagtggtagtggtcatgccatatactacgcagactctgtgaagggccgattcaccatctccagagacaacgccaacaactcactgtatctgcaaatgaacagtctgacagccgaggacacggctgtttattactgtgcgagagaaaagtaccagctactacttggcaagtacgactacggtatggacgtctgggggcgggggaccacggtcaccgtctcgagtggaggcggcggttcaggcggaggtggctctggcggtggcggaagtgcactgcctgtgctgactcagccaccctcagcgtctgggacccccgggcagagggtcaccatctcttgttctggaagcagctccaacatcggaagtaatactttaaactggtaccagcagctcccaggaacggcccccaaactcctcatctatagtaatgatcagcggccctcaggggtccctgaccgattctctggctccaagtctggcacctcagcctccctggccatcagtgggctccagtctgaggatgaggctgattattactgtgcagcatgggatgacagcctgaatggctgggtgttcggcggagggaccaaggtcaccgtcctaggtgcggccgc

SEQ ID NO: 69 - DNA, 698 bp, Homo sapiens
agggcctctggggggacctccagcagttctgctttcagctgggtgcgacaggcccctggacaggggcttcagtggatgggagggatcatccctctctttggtgcagcaaactacgcacaaaaggtccgggccggactcacgattaccgcggacgagtccacgggcacgtcttacatgaaactggaaaatttgcagtctgacgacacggccgtttatttctgtgcgactaacggacagacgaggtcgccacccggctactactacggcatggacgtctggggccgaggcaccctggtcaccgtctcgagtggaggcggcggttcaggcggaggtggctctggcggtggcggaagtgcacagtctgtgttgacgcagctgccctcagcgtctggggcccccgggcagaggatcaccatctcttgttccggaagcaccttcaacatcgggagaaattatgttgactggtataaacaactccccggaacggcccccaaactcttcatctataagaatgatcagcgaccctcaggggtccctggccgattctctggctccaagtctggcacctcagcctccctggtcgtaagtggactccgctccgaggatgaggctgattattactgtctgacttgggatgacagcctgagtggtccggtgttcggcggggggaccaagctgaccgtcctaggtgcggccgc

SEQ ID NO: 70 - DNA, 740 bp, Homo sapiens
gcctgcaagggttttggttacaccttcgtcgatcatggaattagttgggtgcgacaggcccctggacaagggcttgagtggatgggatggatcaacactcacgacggtcacacaaactatgcacaaaagacacaggccagactcaccatgaccacagatgcctccattaatacttcctacatggagctgcggagcctgacatctgacgacacggccgtctattattgtgcccgggggggagagactcggaccgcacatagatctcgcagggccacgaacgacaatggatatccctattactcctctggtctggacgtctggggccaaggaaccctggtcaccgtctcgagtggaggcggcggttcaggcggaggtggctctggcggtggcggaagtgcacaggctgtgctgactcagccgtcctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggttgggtgttcggcggagggaccaagctgaccgtcctaggtgcggccgc

SEQ ID NO: 71 - DNA, 736 bp, Homo sapiens
agcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggttcgggcgccagaatggttcggggagttattatagacccctacggtatggacgtctggggccgaggcaccctggtcaccgtctcgagtggaggcggcggttcaggcggaggtggctctggcggtggcggaagtgcacagtctgtgctgactcagccaccctcagcgtctgggacccccgggcagagggtcaccatctcttgttctggaagcagctccaacgtcggaagtaattatgtatcctggtatcagcagttcccaggaacggcccccaaactcctcatctataggaataatcagcggccctcaggggtccctgaccggttctctggctccaagtctggcatttcagcctccctggccatcagtgggctccggtccgaggatgaggctgatttttactgtgtagcatgggatgacagcctgagggaatatgtcttcggaactgggaccaaggtcaccgtcctaggtgcggccgc

SEQ ID NO: 72 - DNA, 741 bp, Homo sapiens
ggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgaaaggtgggactagggtgacccaccggggtggttttgatatatggggccgagggacaatggtcaccgtctcgagtggaggcggcggttcaggcggaggtggctctggcggtggcggaagtgcactgcctgtgctgactcagcccccctcagcgtctggggcccccgggcagaggatcaccatctcttgttccggaagcaccttcaacatcgggagaaattatgttgactggtataaacaactccccggaacggcccccaaactcttcatctataagaatgatcagcgaccctcaggggtccctgaccgattctctggctccaagtctggcacctcagcctccctggtcgtaagtggactccgctccgaggatgaggctgattattactgtctgacttgggatgacagcctgagtggtccggtgttcggcggggggaccaagctgaccgtcctaggtgcggccgc

SEQ ID NO: 73 - protein, 7 aa, Mus sp.
GGKYFDY

SEQ ID NO: 74 - protein, 24 aa, Mus sp.
DSGLQQGPRRRGARVNFSYYGLDV

SEQ ID NO: 75 - protein, 21 aa, Homo sapiens
ERIVPAGLRNRGMVTAVGMDV

SEQ ID NO: 76 - protein, 13 aa, Homo sapiens
GGGRYDSSHGFDS

SEQ ID NO: 77 - protein, 13 aa, Homo sapiens
DWRWQQFGGWFDP

SEQ ID NO: 78 - protein, 10 aa, Homo sapiens
LFRGSGYVRH

SEQ ID NO: 79 - protein, 7 aa, Homo sapiens
HYYYMDV

SEQ ID NO: 80 - protein, 10 aa, Homo sapiens
VPLRFDGFDV

SEQ ID NO: 81 - protein, 17 aa, Homo sapiens
ALHLDYVWRTYNYYFDN

SEQ ID NO: 82 - protein, 24 aa, Homo sapiens
GVTYHYDHDRRGVTAQIYNHGLDV

SEQ ID NO: 83 - protein, 17 aa, Homo sapiens
QGCSGGKCYEKMYASDI

SEQ ID NO: 84 - protein, 17 aa, Homo sapiens
RGSYSNYERGYYYHMDV

SEQ ID NO: 85 - protein, 13 aa, Homo sapiens
LNWNHGPYYGMDV

SEQ ID NO: 86 - protein, 8 aa, Homo sapiens
DPGSLSAF

SEQ ID NO: 87 - protein, 13 aa, Homo sapiens
DWRWQQFGGWFDP

SEQ ID NO: 88 - protein, 16 aa, Homo sapiens
AEDDSRGYWNHYFSDY

SEQ ID NO: 89 - protein, 13 aa, Homo sapiens
DWRWQQFGGWFDP

SEQ ID NO: 90 - protein, 19 aa, Homo sapiens
GGLGRFFDGPSHFSYYMEV

SEQ ID NO: 91 - protein, 9 aa, Homo sapiens
VNNWNAFDQ

SEQ ID NO: 92 - protein, 18 aa, Homo sapiens
DLMRLARRDEYYYYYMDV

SEQ ID NO: 93 - protein, 13 aa, Homo sapiens
DWRWQQFGGWFDP

SEQ ID NO: 94 - protein, 13 aa, Homo sapiens
SSKIGFQVGELDY

SEQ ID NO: 95 - protein, 25 aa, Homo sapiens
GRRRERSINMIRGVRPQYDDSGMDV

SEQ ID NO: 96 - protein, 27 aa, Homo sapiens
NTGKGITLVRGVYCQDCDRSSTSRMDV

SEQ ID NO: 97 - protein, 16 aa, Homo sapiens
DMGYSYGYGTRGLFDY

SEQ ID NO: 98 - protein, 13 aa, Homo sapiens
DWRWQQFGGWFDP

SEQ ID NO: 99 - protein, 17 aa, Homo sapiens
GDGVVAGTTYYYYGMDV

SEQ ID NO: 100 - protein, 18 aa, Homo sapiens
FHDWRHCNSATCQPPFDH

SEQ ID NO: 101 - protein, 18 aa, Homo sapiens
DHRSGRGGGSYLLRPLDY

SEQ ID NO: 102 - protein, 18 aa, Homo sapiens
DQVFYESGSYYIRPSFDF

SEQ ID NO: 103 - protein, 7 aa, Homo sapiens
RRYALDY

SEQ ID NO: 104 - protein, 16 aa, Homo sapiens
EKYQLLLGKYDYGMDV

SEQ ID NO: 105 - protein, 16 aa, Homo sapiens
NGQTRSPPGYYYGMDV

SEQ ID NO: 106 - protein, 28 aa, Homo sapiens
GGETRTAHRSRRATNDNGYPYYSSGLDV

SEQ ID NO: 107 - protein, 19 aa, Homo sapiens
GSGARMVRGVIIDPYGMDV

SEQ ID NO: 108 - protein, 13 aa, Homo sapiens
GGTRVTHRGGFDI

SEQ ID NO: 109 - DNA, 17 bp, Artificial Sequence
  Description of Artificial Sequence Synthetic primer
ccccacgcggttccagc

SEQ ID NO: 110 - DNA, 17 bp, Artificial Sequence
  Description of Artificial Sequence Synthetic primer
tacctattgcctacggc

SEQ ID NO: 111 - DNA, 3918 bp, Artificial Sequence
  Description of Artificial Sequence Synthetic polynucleotide
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctatgcggcccagccggccatggcccaggtccagtcgacaggtggaggcggttcaggcggaggtggctctggcggtggcggaagtgcactcatcaaacggcggccgcaggtgcgccggtgccgtatccggatccgctggaaccgcgtgccgcataggctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcccggcgggtgtggtggttacgcgcagcgtgaccgctacacttgccagcgccctagcccccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgtcctttgacgttcgagtccacgttctttaatagtggactcttgttccaaactggaacaatactcaaccctatctcgggctattcttttgatttataagggattttgccgatttcggcctattggttaaaaaatgagctgatttaacaaaaatttaacgcgaattttaacaaaatattaacgtttacaattttatggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 112 - DNA, 3351 bp, Artificial Sequence
  Description of Artificial Sequence Synthetic polynucleotide
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaaattctatttcaaggagacagtcataatgaaatacctattgcctacggcagccgctggattgttattactcgcggcccagccggccatggcccaggtgcagctgcaggtcggcctcgagatcaaacgggcggccgcaggtgcgccggtgccgtatccagatccgctggaaccgcgtggggccgcaagcgcttggagccacccgcagttcgaaaaataataaggatccgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcccggcgggtgtggtggttacgcgcagcgtgaccgctacacttgccagcgccctagcccccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgtcctttgacgttcgagtccacgttctttaatagtggactcttgttccaaactggaacaatactcaaccctatctcgggctattcttttgatttataagggattttgccgatttcggcctattggttaaaaaatgagctgatttaacaaaaatttaacgcgaattttaacaaaatattaacgtttacaattttatggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 113 - DNA, 20 bp, Artificial Sequence
  Description of Artificial Sequence Synthetic primer
ctggatggtgggaagatgga

SEQ ID NO: 114 - DNA, 20 bp, Artificial Sequence
  Description of Artificial Sequence Synthetic primer
tcagaggaaggaaacagggt

SEQ ID NO: 115 - DNA, 30 bp, Artificial Sequence
  Description of Artificial Sequence Synthetic primer
cttacaaccacaatccctgggcacaatttt

SEQ ID NO: 116 - DNA, 24 bp, Artificial Sequence
  Description of Artificial Sequence Synthetic primer
ctttgtgggccctctgggctcaat

SEQ ID NO: 117 - DNA, 24 bp, Artificial Sequence
  Description of Artificial Sequence Synthetic primer
tgaaatgggcccgctgggctcaag

SEQ ID NO: 118 - DNA, 24 bp, Artificial Sequence
  Description of Artificial Sequence Synthetic primer
gggcttgggtattctaggctcgat

SEQ ID NO: 119 - DNA, 23 bp, Artificial Sequence
  Description of Artificial Sequence Synthetic primer
gaggtgcagcttcaggagtcagg

SEQ ID NO: 120 - DNA, 23 bp, Artificial Sequence
  Description of Artificial Sequence Synthetic primer
caggtgcagctgaaggagtcagg

SEQ ID NO: 121 - DNA, 23 bp, Artificial Sequence
  Description of Artificial Sequence Synthetic primer
gaggtccagctgcaacagtctgg

SEQ ID NO: 122 - DNA, 23 bp, Artificial Sequence
  Description of Artificial Sequence Synthetic primer
gaggttcagctgcagcagtctgg

SEQ ID NO: 123 - DNA, 23 bp, Artificial Sequence
  Description of Artificial Sequence Synthetic primer
caggtccaactgcagcagcctgg

SEQ ID NO: 124 - DNA, 23 bp, Artificial Sequence
  Description of Artificial Sequence Synthetic primer
caggttcagctgcagcagtctgg

SEQ ID NO: 125 - DNA, 23 bp, Artificial Sequence
  Description of Artificial Sequence Synthetic primer
gaggtgaagctggtggagtctgg

SEQ ID NO: 126 - DNA, 23 bp, Artificial Sequence
  Description of Artificial Sequence Synthetic primer
gaggtgaagctggtggaatctgg

SEQ ID NO: 127 - DNA, 23 bp, Artificial Sequence
  Description of Artificial Sequence Synthetic primer
gaggttcagcttcagcagtctgg

SEQ ID NO: 128 - DNA, 24 bp, Artificial Sequence
  Description of Artificial Sequence Synthetic primer
tgaggagacggtgaccgtggtccc

SEQ ID NO: 129 - DNA, 24 bp, Artificial Sequence
  Description of Artificial Sequence Synthetic primer
tgaggagactgtgagagtggtgcc

SEQ ID NO: 130 - DNA, 24 bp, Artificial Sequence
  Description of Artificial Sequence Synthetic primer
tgcagagacagtgaccagagtccc

SEQ ID NO: 131 - DNA, 24 bp, Artificial Sequence
  Description of Artificial Sequence Synthetic primer
tgaggagacggtgactgaggttcc

SEQ ID NO: 132 - DNA, 23 bp, Artificial Sequence
  Description of Artificial Sequence Synthetic primer
gacattgtgatgacacagtctcc

SEQ ID NO: 133 - DNA, 23 bp, Artificial Sequence
  Description of Artificial Sequence Synthetic primer
gatgttgtgatgacccaaactcc

SEQ ID NO: 134 - DNA, 23 bp, Artificial Sequence
  Description of Artificial Sequence Synthetic primer
gatatccagatgacacagactcc

SEQ ID NO: 135 - DNA, 23 bp, Artificial Sequence
  Description of Artificial Sequence Synthetic primer
caaattgttctcacccagtctcc

SEQ ID NO: 136 - DNA, 23 bp, Artificial Sequence
  Description of Artificial Sequence Synthetic primer
caggctgttgtgactcaggaatc

SEQ ID NO: 137 - DNA, 24 bp, Artificial Sequence
  Description of Artificial Sequence Synthetic primer
tttgatttccagcttggtgcctcc

SEQ ID NO: 138 - DNA, 24 bp, Artificial Sequence
  Description of Artificial Sequence Synthetic primer
ttttatttccagcttggtcccccc

SEQ ID NO: 139 - DNA, 24 bp, Artificial Sequence
  Description of Artificial Sequence Synthetic primer
tttcagctccagcttggtcccagc

SEQ ID NO: 140 - DNA, 24 bp, Artificial Sequence
  Description of Artificial Sequence Synthetic primer
acctaggacagtgaccttggttcc

SEQ ID NO: 141 - DNA, 56 bp, Artificial Sequence
  Description of Artificial Sequence Synthetic primer
gtcctcgcaactgcggcccagccggccatggccgaggtgcagcttcaggagtcagg

SEQ ID NO: 142 - DNA, 56 bp, Artificial Sequence
  Description of Artificial Sequence Synthetic primer
gtcctcgcaactgcggcccagccggccatggcccaggtgcagctgaaggagtcagg

SEQ ID NO: 143 - DNA, 56 bp, Artificial Sequence
  Description of Artificial Sequence Synthetic primer
gtcctcgcaactgcggcccagccggccatggccgaggtccagctgcaacagtctgg

SEQ ID NO: 144 - DNA, 56 bp, Artificial Sequence
  Description of Artificial Sequence Synthetic primer
gtcctcgcaactgcggcccagccggccatggccgaggttcagctgcagcagtctgg

SEQ ID NO: 145 - DNA, 56 bp, Artificial Sequence
  Description of Artificial Sequence Synthetic primer
gtcctcgcaactgcggcccagccggccatggcccaggtccaactgcagcagcctgg

SEQ ID NO: 146 - DNA, 56 bp, Artificial Sequence
  Description of Artificial Sequence Synthetic primer
gtcctcgcaactgcggcccagccggccatggcccaggttcagctgcagcagtctgg

SEQ ID NO: 147 - DNA, 56 bp, Artificial Sequence
  Description of Artificial Sequence Synthetic primer
gtcctcgcaactgcggcccagccggccatggccgaggtgaagctggtggagtctgg

SEQ ID NO: 148 - DNA, 56 bp, Artificial Sequence
  Description of Artificial Sequence Synthetic primer
gtcctcgcaactgcggcccagccggccatggccgaggtgaagctggtggaatctgg

SEQ ID NO: 149 - DNA, 56 bp, Artificial Sequence
  Description of Artificial Sequence Synthetic primer
gtcctcgcaactgcggcccagccggccatggccgaggttcagcttcagcagtctgg

SEQ ID NO: 150 - DNA, 36 bp, Artificial Sequence
  Description of Artificial Sequence Synthetic primer
gagtcattctcgtgtcgacacggtgaccgtggtccc

SEQ ID NO: 151 - DNA, 36 bp, Artificial Sequence
  Description of Artificial Sequence Synthetic primer
gagtcattctcgtgtcgacactgtgagagtggtgcc

SEQ ID NO: 152 - DNA, 36 bp, Artificial Sequence
  Description of Artificial Sequence Synthetic primer
gagtcattctcgtgtcgacacagtgaccagagtccc

SEQ ID NO: 153 - DNA, 36 bp, Artificial Sequence
  Description of Artificial Sequence Synthetic primer
gagtcattctcgtgtcgacacggtgactgaggttcc

SEQ ID NO: 154 - DNA, 41 bp, Artificial Sequence
  Description of Artificial Sequence Synthetic primer
tgagcacacagtgcactcgacattgtgatgacacagtctcc

SEQ ID NO: 155 - DNA, 41 bp, Artificial Sequence
  Description of Artificial Sequence Synthetic primer
tgagcacacagtgcactcgatgttgtgatgacccaaactcc

SEQ ID NO: 156 - DNA, 41 bp, Artificial Sequence
  Description of Artificial Sequence Synthetic primer
tgagcacacagtgcactcgatatccagatgacacagactcc

SEQ ID NO: 157 - DNA, 41 bp, Artificial Sequence
  Description of Artificial Sequence Synthetic primer
tgagcacacagtgcactccaaattgttctcacccagtctcc

SEQ ID NO: 158 - DNA, 41 bp, Artificial Sequence
  Description of Artificial Sequence Synthetic primer
tgagcacacagtgcactccaggctgttgtgactcaggaatc

SEQ ID NO: 159 - DNA, 48 bp, Artificial Sequence
  Description of Artificial Sequence Synthetic primer
gagtcattctcgacttgcggccgctttgatttccagcttggtgcctcc

SEQ ID NO: 160 - DNA, 48 bp, Artificial Sequence
  Description of Artificial Sequence Synthetic primer
gagtcattctcgacttgcggccgcttttatttccagcttggtcccccc

SEQ ID NO: 161 - DNA, 48 bp, Artificial Sequence
  Description of Artificial Sequence Synthetic primer
gagtcattctcgacttgcggccgctttcagctccagcttggtcccagc

SEQ ID NO: 162 - DNA, 48 bp, Artificial Sequence
  Description of Artificial Sequence Synthetic primer
gagtcattctcgacttgcggccgcacctaggacagtgaccttggttcc

(19) United States
(12) Patent Application Publication
(10) Pub. No.: US 2012/0058117 A1
(43) Pub. Date: Mar. 8, 2012

(54) NEUROPILIN-1 INHIBITORS

(75) Inventors: Christine Margarete UNGER, Muenchen (DE); Gerald BESTE, Muenchen (DE); Carolin ZEHETMEIER, Muenchen (DE); Blanca LAIN, Brighton, MA (US); Claudia TORELLA, Muenchen (DE); Jens NIEWÖHNER, Muenchen (DE); Daniel G. JAY, Brighton, MA (US); Brenda K. EUSTACE, Brookline, MA (US); Roland KNAUER, Muenchen (DE); Kristian Hobold JENSEN, Muenchen (DE)

(73) Assignees: MEDAREX, INC., Princeton, NJ (US); TUFTS UNIVERSITY, Boston, MA (US)

(21) Appl. No.: 13/166,581

(22) Filed: Jun. 22, 2011

Related U.S. Application Data
(63) Continuation of application No. 10/539,402, filed on Dec. 22, 2005, now abandoned.
(60) Provisional application No. 60/435,893, filed on Dec. 20, 2002.

(30) Foreign Application Priority Data
Jan. 15, 2003 (EP) 03000615.9

(51) Int. Cl. A61K 39/395; C07K 14/00; C12N 9/96; G01N 33/574; G01N 33/566; C12N 5/071; C12N 5/09; A61K 38/02; C12N 15/13; C12N 15/11; C12N 15/63; C40B 30/04; A61P 35/04; A61P 35/00; C07K 16/28

(52) U.S. Cl. 424/135.1; 530/387.3; 530/389.7; 530/350; 530/391.1; 530/391.3; 435/188; 435/7.23; 435/7.92; 435/375; 424/174.1; 514/1.1; 536/23.53; 536/23.1; 435/320.1; 506/9; 514/44 R; 514/19.8; 514/19.3

(57) ABSTRACT

The present invention relates to molecules interfering with the function of neuropilin-1 in the context of angiogenesis and the treatment of cancer. Molecules, polypeptides, antibodies, compositions and methods are provided that are useful for reducing, inhibiting or treating angiogenesis, the invasion of blood vessels into tumors, and/or the invasion or the metastatic potential of specific tumor cells. Additionally, a method is provided that allows identifying molecules, which interfere with the functionality of neuropilin-1. Furthermore, a method is provided that allows determining whether a naturally occurring tumor cell depends on functional neuropilin-1 for its invasiveness and/or metastatic potential.

The present invention relates to molecules interfering with the function of neuropilin-1 in the context of angiogenesis and the treatment of cancer. Molecules, polypeptides, antibodies, compositions and methods are provided that are useful for reducing, inhibiting or treating angiogenesis, the invasion of blood vessels into tumors, and/or the invasion or the metastatic potential of specific tumor cells. Additionally, a method is provided that allows identifying molecules, which interfere with the functionality of neuropilin-1. Furthermore, a method is provided that allows determining whether a naturally occurring tumor cell depends on functional neuropilin-1 for its invasiveness and/or metastatic potential.

### BACKGROUND OF THE INVENTION

Malignant tumors shed cells, which migrate to new tissues and create secondary tumors. The process of generating secondary tumors is called metastasis and is a complex process in which tumor cells colonize sites distant from the primary tumor. Recently, research has been focused on identifying specific proteins involved in metastasis, which can be used as a basis for better diagnostic or improved therapeutic strategies. Cell adhesion molecules (CAM's), which mediate cell-cell or cell-matrix interactions, have been proposed to be involved in the process of metastasis. Cell adhesion in normal cells involves interactions between numerous cell surface proteins. Adhesive interactions are known to involve interactions between substances surrounding the cell (e.g., extracellular matrix molecules, for example fibronectin, vitronectin and laminin) and extracellular adhesion receptors. It has also become apparent that cell adhesion molecules fulfill much more complex functions, which may result in cells acquiring the ability to proliferate and invade host tissues.

Liotta (1986) Cancer Res. 46, 1-7 has proposed a three-step hypothesis for the process of metastasis: The first step is tumor cell attachment via cell surface receptors. The anchored tumor cell next secretes hydrolytic enzymes or induces host cells to secrete enzymes, which can degrade the matrix locally. Matrix lysis most likely takes place in a highly localized region close to the tumor cell surface. The third step is tumor cell locomotion into the region of the matrix modified by proteolysis. Thus, invasion of the matrix is not merely due to passive growth pressure but requires active biochemical mechanisms. Degradation of the surrounding normal tissue is a central feature of invasiveness of malignant tumors.

The growth of a tumor or metastasis can also be described as the growth of undifferentiated cells that divide rapidly, and expand three-dimensionally to create a new mass of tissue. The cells within this mass require oxygen and nutrients. As the tumor grows, many cells within it are too far from the nearest blood vessel to maintain adequate concentrations of oxygen. When oxygen levels are decreased (a condition known as hypoxia), certain genes are activated. One of these genes, hypoxia-inducible factor 1 (HIF-1) in turn binds to the vascular endothelial growth factor (VEGF) gene and helps to activate VEGF gene expression. In addition to hypoxia, other factors can activate VEGF expression in tumor cells. For example, certain hormones or growth factors have been shown to activate VEGF expression, and some studies suggest that the activation of certain oncogenes or the inactivation of certain tumor suppressor genes may also lead to VEGF gene expression.

The VEGF protein is secreted by tumor cells and activates VEGF receptors on vascular endothelial cells. These cells begin to form new blood vessels into the tumor, providing a supply of oxygen and nutrients that support tumor growth.

This process of the growth of new vessels from pre-existing vasculature is called angiogenesis and generally occurs during the development of all tissues.

Angiogenesis is understood herein, furthermore, as the sprouting of new blood vessels, which is dependent on endothelial cell proliferation and migration. It occurs at specific times in development and growth, e.g. during development of the embryo or wound healing. (Folkman et al. (1987) Science 235, 442-447, Folkman (1991) J. Natl. Cancer Inst. 82, 4-6).

Moreover, it has been shown that angiogenesis is involved in the Pathogenesis of disorders dependent on the growth of new blood vessels, the most relevant of which are tumor growth and growth of metastases (Hanahan et al. (1996) Cell 86, 353-364).

Vascular endothelial growth factor (VEGF) is considered to be the prime regulator for both physiological and pathological angiogenesis, acting through VEGF receptors on endothelial cells and mediating angiogenic signals (Dvorak et al. to (1999) Curr. Top. Microbial. Immunol. 237, 97-132, Neufeld et al. (1999) FASEB J. 13, 9-22).

Angiogenesis depends essentially on the expression of vascular endothelial growth factors to stimulate endothelial cell growth and formation of new blood vessels. This family of proteins consists of six members (VEGF A-E and PIGF) of which VEGF-A is the most important for angiogenesis. There are seven known splice variants of VEGF-A of which six are pro-angiogenic. The VEGF isoforms that are produced by alternative splicing from a single gene containing nine exons (Ferrara, et al., Endocr. Rev., 13:18-32 (1992); Tischer, et al., J. Biol. Chem., 806:11947-11954 (1991); Ferrara, et al., Trends Cardio Med., 3:244-250 (1993); Polterak, et al., J. Biol. Chem., 272:715 1 7158 (1997)). Human VEGF isoforms consists of monomers of 121, 145, 165, 189, and 206 amino acids, each capable of making an active homodimer (Polterak et al., J Biol. Chem, 272:7151-7158 (1997); Houck, et al., Mol. Endocrinol., 8:1806-1814 (1991)). The VEGF121 and VEGF165 isoforms are the most abundant.

As said before, VEGF stimulates specifically VEGF receptors and thereby induces VEGF receptor tyrosine kinases, KDR/Flk-1 and/or Flt-1, which are mostly expressed by endothelial cells (EC) (Terman, et al., Biochem. Biophys. Res. Commun., 187:1579-1586 (1992); Shibuya, et al., Oncogene, 5:519-524 (1990); De Vries, et al., Science, 265:989-991 (1992); Gitay-Goran, et al., J. Biol. Chem., 2 87:6003-6096 (1992); Jakernan, et al., J Clin. Invest., 89:244-253 (1992)).

It appears that VEGF activities such as mitogenicity, chernotaxis, and induction of morphological changes are mediated by KDR/Flk-I but not Flt-1, even though both receptors undergo phosphorylation upon binding of VEGF (Millauer, et al., Cell, 72:835-846 (1993); Waltenberger, et al., J Biol. Chem., 269:26988-26995 (1994); Seetharam, et al., Oncogene, 10: 135-147 (1995); Yoshida, et al., Growth Factors, 7:131-138 (1996)). Recently, Soker et al., identified a new VEGF receptor which is expressed on EC and various tumor-derived cell lines such as breast cancer-derived MDA-MB-231 (23 1) cells (Soker, et al., J. Biol. Chem., 271:5761-5767 (1996)).

This receptor requires the VEGF isoform to contain the portion encoded by exon 7. For example, although both VEGF121 and VEGF 165 bind to KDR/Flk-I and Flt-1, only VEGF 165 binds to the new receptor.

Thus, this it seems that the new receptor is an isoform-specific receptor. It has been named the VEGF165 receptor (VEGF165R). It will also bind the 189 and 206 isoforms. VEGF165R has a molecular mass of approximately 130 kDa, and it binds VEGF165 with a Kd of about 2×10⁻¹⁰ M, compared with approximately 5×10⁻¹² M for KDR/Flk-1. In structure-function analysis, it was shown directly that VEGF 165 binds to VEGFI65R via its exon 7 encoded domain, which is absent in VEGF121 (Soker, et al., J Biol. Chem., 271:57615767 (1996)).

By isolating the VEGF165R related DNA it was discovered that this novel VEGF receptor is structurally unrelated to Flt-I or KDR/Flk-I and is expressed not only by endothelial cells but also by non-endothelial cells, including tumor cells.

Furthermore, in ascertaining the function of the VEGF165R it has been discovered that this receptor has been identified as a cell surface mediator of neuronal cell guidance and called neuropilin (Kolodkin et al., Cell 90:753-762 (1997)).

Neuropilin-1 is a transmembrane glycoprotein with a mass of approximately 130 kDa. Neuropilin-1 is a multifunctional protein. Fujisawa et al. (1998) Cur. Opin. Neurobiol. 8, 587-592 describe that neuropilin-1 acts as a receptor for the semaphorin/collapsin family of neural guidance mediators. Furthermore, the expression of neuropilin-1 in endothelial cells enhances the binding of VEGF165 to VEGFR-2 and VEGF165-induced chemotaxis. According to Gagnon et al. and WO 99/29729, neuropilin-1 acts as a co-receptor that enhances VEGFR-2 activity (Gagnon et al. (2000) PNAS 97, 2573-2578).

Neuropilin-1 is also expressed on certain tumor cells of ektodermal origin, like cells derived from prostate and breast carcinoma as well as from melanoma. In experiments with breast cancer cells VEGF₁₆₅ stimulated breast cancer cell motility in a dose-dependent manner (WO 99/29729). A comparison between two types of rat prostate carcinoma cells, AT2.1 cells and AT3.1 cells showed that the highly motile AT3.1 cells expressed more neuropilin-1 than the less motile AT2.1 cells. Collapsin-1, a ligand of neuropilin-1, inhibited the basal migration of PAE cells (porcine aortic endothelial cells) when cells were transfected with the neuropilin-1 cDNA to overexpress neuropilin-1. All this was interpreted by the authors of WO 99/29729 to suggest that neuropilin-1 expression was associated with a motile phenotype of tumor cells. However, it is dangerous and speculative to assign a physiological role for a protein based on a study in which it has been overexpressed, and it is an accepted standard in science to interpret results of overexpression studies with great care.

As discussed in WO99/29729 as well as in Soker et al., Cell (1998) 92, 735-745, neuropilin-1 can act as a co-receptor of VEGFR-2 for VEGF₁₆₅ to mediate the effects of this regulator for both physiological and pathological angiogenesis. VEGFR-2 is expressed in endothelial and haematopoietic precursors, endothelial cells, nascent haematopoietic stem cells and the umbilical cord stroma only (for review see Robinson and Stringer J. Cell Sci. (2001) 114, 853-865). If the cancer-related function of neuropilin-1 was dependent on its role in VEGF-signaling—as suggested by, e.g., WO 99/29729—then this cancer-related function of neuropilin-1 should only be relevant for tissues which express neuropilin-1 and VEGFR-2 together in the presence of VEGF. The expression of VEGFR-2 is, however, limited to certain tissues. It is therefore unclear whether neuropilin-1 plays any cancer-related role in those tissues, which do not express VEGFR-2. The authors of WO 99/29729 showed that VEGF₁₆₅ stimulated 231 breast cancer cell motility in a dose dependent-manner. They postulated that tumor cells, which do not express the VEGF receptors KDR or Flt-1, are responsive to VEGF₁₆₅ via the neuropilin-1.

The determination of the physiological role of a protein is a prerequisite for deciding whether interference with this protein's function might be a possible avenue for the treatment of disease or not. It must be kept in mind that in a physiological setting, that is to say for example in a naturally occurring tumor cell of a patient, neuropilin-1 is overexpressed together with other proteins which can modulate and change the function of neuropilin-1. It is the functional interplay between neuropilin-1 and interacting proteins that determines its physiological role.

Accordingly, it was an object of the present invention to identify and provide further molecules that bind to neuropilin-1 and modulate the neuropilin-1 function and can thus be used to further characterise neuropilin-1 function, but also can be used to actively interfere and modulate the neuropilin-1 function e.g. in medical treatment or therapeutic set ups.

### SUMMARY OF THE INVENTION

In an unbiased screen for molecules that can inhibit angiogenesis of endothelial cells as well as the invasion and/or adhesion of tumor cells surprisingly a poly-peptide, particularly an antibody fragment binding to the extracellular domain of neuropilin-1 has been identified as such an inhibitor.

The present invention relates to neuropilin binders, e.g. polypeptides, antibodies, antibody fragments, single chain antibodies (scFv) or bioconjugates, which can specifically bind to the extracellular domain of neuropilin-1 and inhibit neuropilin-1 function.

In a further embodiment the neuropilin binders (NPBs) are characterised by the additional feature, that they are capable of modulating or inhibiting neuropilin related functions, but can not interfere or inhibit VEGF/neuropilin-1 interaction.

The NPBs of the invention can be selected from the group comprising the sequences SEQ ID No: 1, 2, 5 to 38.

Furthermore, the NPB of the invention can be labeled with detectable groups, if desired.

The invention further relates to pharmaceutical compositions comprising the NPB or a bioconjugate comprising the NPB of the invention.

In a further embodiment the invention relates to nucleic acid molecules encoding the NPB of the invention, as well as to vectors comprising such a nucleic acid and to host cells comprising such a vector.

In a further embodiment the invention relates to the use of NPB as molecules inhibiting neuropilin-1 function for the manufacture of a medicament for the treatmeat or prevention of invasion and/or metastasis of naturally occurring cancer cells; e.g. of mesodermal origin, wherein invasiveness and/or metastatic potential of said cancer cells depends on neuropilin-1 function.

In a further embodiment the invention relates to a method of treating or preventing invasion and/or metastasis in a patient, wherein the invasiveness and/or metastatic potential of said cancer cells depends on neuropilin-1 function.

In still a further embodiment the invention relates to the NPBs of the invention as medicament or the use of the NPB of the invention in the manufacture of a medicament for the treatment or prevention of neuropilin-1-dependent angiogenesis and non-physiological blood vessel growth, particularly correlated with a tumor, wherein said NPBs do not interfere or inhibit the interaction between neuropilin-1 and VEGF₁₆₅.

In a further embodiment the invention relates to a method to determine the dependency of the invasiveness and/or adhesiveness of a naturally occurring cancer cell on the functionality of neuropilin-1.

In a further embodiment the invention relates to a method for the identification of a ligand useful for inhibiting the invasiveness and/or adhesiveness of a naturally occurring cancer cell, particularly the identification of such ligands that bind to the extracellular domain of neuropilin-1.

In still a further embodiment the invention relates to a method for the identification of a ligand useful for inhibiting the angiogenesis of endothelial cell, particularly the identification of such ligands that bind to the extracellular domain of neuropilin-1.

### DETAILED DESCRIPTION OF THE INVENTION

The present invention now shows that neuropilin-1-dependent angiogenesis, invasion and/or adhesion can be inhibited by using the molecules of this invention. Surprisingly, it has surprisingly found that stimulation of HT1080 cells, a human sarcoma cell line, with VEGF₁₆₅ has no influence on the invasiveness of cells. Invasiveness of tumor cells (e.g HT1080 cells) could only be stimulated in the presence of FCS, whereas the migration ability of HUVEC cells, primary human umbilical vein endothelial cells, could indeed be stimulated with VEGF₁₆₅.

Furthermore, it has been found that several molecules of this invention inhibit the tube formation of HUVEC cells, an assay used to study angiogenesis. The present inventions shows for the first time that some of the neuropilin binders (NPBs) that inhibit angiogenesis do not to interfere or inhibit the NP-1/VEGF₁₆₅ interaction. Results show further that these NPBs bind to a different epitope of NP-1 than VEGF₁₆₅.

The result show that neuropilin-1 acts as an active mediator of the invasion and/or adhesion of metastatic tumor cells and furthermore has a functional role angiogenesis, whereby the NPBs do not interfere or inhibit the NP-1/VEGF₁₆₅ interaction. This opens, for the first time, the possibility to develop drugs, which inhibit metastasis of tumor cells—and especially of metastatic sarcomas, which are hardly curable with current cancer treatments—wherein the metastatic potential depends on neuropilin-1-dependent influence on angiogenesis, invasion and/or adhesion.

In order that the invention described herein may be more fully understood, the following detailed description and definitions are provided. As used herein, the following definitions shall apply unless otherwise indicated.

A “polypeptide” as used herein is a molecule comprising more than 10, preferably more than 20, most preferably more than 30, and less than 10000, more preferably less than 2500, most preferably less than 1000 amino acids. Also polypeptides with substantial amino acid sequence identity and polypeptides, which contain a low percentage of modified or non-natural amino acids, are encompassed.

In the wording of the present invention the term polypeptide generally covers also the terms antibodies, antibody fragments or single chain antibodies (scFv).

The terms “antibody” and “immunoglobulin”, as used herein refer to any immunological binding agent, including polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. The heavy-chain constant domains that correspond to the different classes of immunoglobulin are termed alpha, delta, epsilon, gamma and mu, respectively. The subunit structures and three-dimensional configurations of different classes of immunoglobulin are well known.

Antibodies may be also selected from modified immunoglobulin, for example chemically or recombinantly produced antibodies, CDR (complementary determining region) grafted antibodies or humanized antibodies, site directed mutagenized antibodies that exhibit substantial amino acid sequence identity in their CDR regions, particularly in their CDR3 region, to the corresponding antibody fragments of the invention and retain substantially the same affinity for neuropilin-1 binding as the corresponding antibody fragments.

The CDRs (complementary determining region) of an antibody are the parts of these molecules, that determine their specificity and make contact with specific ligands. The CDRs are the most variable parts of the molecule and contribute to the diversity of these molecules. They are structurally defined in a human IgG as amino acids 24 to 41 (CDR-L1), 50 to 57 (CDR-L2) and 90 to 101 (CDR-L3) of the light chain and amino acids 26 to 38 (CDR-H1), 51 to 70 (CDR-L2) and 100 to 125 (CDR-H3) of the heavy chain (see Kabat et al. (1987) 4th edn US Department of Health and Human Services, Public Health Service, NIH, Bethesda). The CDR regions of an antibody fragment can easily be determined by somebody skilled in the art by aligning the antibody fragment with said human IgG, e.g. using a program of the NCBI that allows to “Blast”, and thereby align, two sequences with one another, and identifying the amino acids of the antibody fragment corresponding to the CDRs of a human IgG.

Substantial amino acid sequence identity as used herein means that at least 70%, preferably at least 75%, 80%, 85%, 90%, more preferably all but 5, still more preferably all but 3 and even more preferably all but 1 of the amino acids of two aligned amino acid sequences, particularly of aligned CDRs, are identical.

The term “antibody fragment” is used to refer to any fragment of an antibody-like molecule that has an antigen binding region, and this term includes antibody fragments such as scFv, dsFv, Fab′, Fab, F(ab′)₂, Fv, single domain antibodies (DABs), diabodies, and the like. The techniques for preparing and using various antibody-based constructs and fragments are well known in the art (see Kabat et al. (1991) J. Immunol. 147, 1709-19), specifically incorporated herein by reference.

“scFv” antibody fragments comprise the VH and VL domains of an antibody, wherein these domains are present in a single polypeptide chain. Generally, the scFv polypeptide further comprises a polypeptide linker between the VH and VL domains that enables the scFv to form the desired structure for antigen binding.

A “Fv” fragment is the smallest antibody fragment that retains an intact antigen binding site.

A “dsFv” is a disulfide stabilized Fv.

A “Fab” fragment, is an antigen binding fragment, containing complete light chains paired with the VH and CH1 domains of the heavy chain.

A “Fab”' fragment, is a reduced F(ab′)₂ fragment.

A “F(ab′)₂” fragment, is a bivalent fragment comprising two Fab fragments linked by a disulfide bridge at the hinge region.

A “single domain antibody (DAB)” is an antibody with only one (instead of two) protein chain derived from only one of the domains of the antibody structure. Dabs exploit the finding that, for some antibodies, half of the antibody molecule binds to its target antigen almost as well as the whole molecule (Davies et al. (1996) Protein Eng. 9: 531-537.

“Diabodies” are bivalent or bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (Holliger et al. (1993) Proc. Natl. Acad. Sci. USA, 90, 6444-6448).

The terms “label” or “labeled” refers to a detectable marker or the incorporation of such, respectively, e.g., by incorporation of a fluorophore-, chromophore- or radio-labeled amino acid or attachment of a fluorophore-, chromophore- or radio-label to a polypeptide or attachment of moieties that can be detected by a labeled second molecule containing a fluorescent marker or enzymatic activity that can be detected by an optical or a colorimetric method. An example for such a two-step detection system is the well-known biotin-avidin system. Various methods of labeling polypeptides and glycoproteins are known in the art and may be used (for example see Lobl et al. (1988) Anal. Biochem., 170, 502-511).

An “epitope” includes any protein determinant capable of specific binding to an immunoglobulin or an antibody fragment. Epitopic determinants usually consist of chemically active surface groupings of molecules such as exposed amino acids, aminosugars, or other carbohydrate side chains and usually have specific three-dimensional structural characteristics, as well as specific charge characteristics.

A “naturally occurring cancer cell” as used herein is a cell that has not been transfected, transduced or otherwise genetically engineered in the laboratory. Such a cell does not comprise artificial DNA sequences, e.g. of vectors, or DNA sequences being found only in other species, but not usually in the species from which the naturally occurring cancer cell was derived. However, a naturally occurring cancer cell may comprise sequences that are not usually found in the species from which it was derived, if those sequences have arisen due to the processes of mutation and selection that took place within the individuum from which the naturally occurring cancer cell was derived, and/or during continued culture of the naturally occurring cancer cell.

Selected cancer cell-types as used herein consist of tumor cells derived from, e.g. the mesoderm, preferably derived from the group of neoplasms consisting of soft tissue tumors and sarcomas, fibromatous neoplasms, myxomatous neoplasms, lipomatous neoplasms, myomatous neoplasms, complex mixed and stromal neoplasms, synovial-like neoplasms, mesothelial neoplasms, lymphatic vessel tumors, osseous and chondromatous neoplasms, giant cell tumors, miscellaneous bone tumors, odontogenic tumors, hodgkin's and non-Hodgkins's lymphoma, plasma cell tumors, mast cell tumors, immunoproliferative diseases and leukemias, in particular tumor cells derived from the group of neoplasms consisting of soft tissue tumors and sarcomas, fibromatous neoplasms, myxomatous neoplasms, lipomatous neoplasms, myomatous neoplasms, complex mixed and stromal neoplasms, synovial-like neoplasms, mesothelial neoplasms, lymphatic vessel tumors, osseous and chondromatous neoplasms, giant cell tumors, miscellaneous bone tumors, odontogenic tumors, in particular tumor cells derived from sarcomas, in particular derived from sarcomas of the bone or sarcomas of soft tissue. Neoplasmas can also be derived from brain, central nervous system, lungs, stomach, lower intestine, colon, liver, kidneys, prostate and the pancreas.

In one embodiment sarcomas can be all sarcomas with the exception of angiomas and haemangiomas.

“Treating metastatic tumors” or “treating micro-metastases”, as used herein means that the metastasis of the tumor is stabilized, prevented, delayed, or inhibited by the molecule of the invention, either as a single medicament or in combination with other medicaments. Stable disease or “No Change” (NC) is a description for the course of the disease with either no change of the metastases or a reduction of less than 50% or an increase of less than 25% over at least 4 weeks. Prevention can be, for example, that no new metastases are detected after the treatment is initiated. This can lead to a two- to three-fold median and/or a %-year survival rate of treated patients compared with untreated patients. A delay can signify a period of at least 8 weeks, 3 months, 6 months or even one year in which no new metastases are detected after the treatment is initiated. Inhibition can mean that the average size or the total number of new metastases is at least 30%, 40%, 50%, 60%, 70%, 80% or even 90% lower in a group treated with the molecule of the invention in comparison with an untreated group. Number, size and prevalence of metastases can be detected by a skilled practitioner in the field of oncology following generally accepted practice and diagnostic procedures for the detection of metastases, for example as outlined in. Harrison Principles of Internal Medicine 15^{th} ed 2001 Mc Graw Hill.

“Metastatic tumors” as used herein includes both tumors at the primary site capable of metastasizing and metastasized tumors ata secondary site. Such metastatic tumors can be of any organ or tissue origin, like brain, central nervous system, lungs, stomach, lower intestine, liver, kidneys or the pancreas, etc., in particular tissue of mesodermal origin such as bone, spleen, thyroid, endometrial, ovarian, or lymphoid tissue.

A micro-metastase is an accumulation of tumor cells with a size smaller than 2 mm, which can usually only be detected by histological methods.

“Angiogenesis” as used herein refers to the ability of endothelial cells to form tubes and eventually, elaborate blood vessels. The angiogenic potential of cells or in other words the inhibition of angiogenesis can be assayed in a so-called tube formation assay as described in Example 16. Inhibition of angiogenesis is seen and measured in correlation to the capacity of cells to form or not to form closed polygons as well as the status of aggregation of cells. As demonstrated in Example 16 and FIG. 14 a significant aggregation of cells and the lack of closed polygon formation strongly correlates with the tube formation of endothelial cells.

“Invasiveness” as used herein is the ability of a cell to migrate through a layer of other cells or to migrate through the extracellular matrix. Invasiveness can be assessed by the Matrigel assay described in Example 8 or Example 9. Invasion is measured as cells that reach the lower surface of the filter during a certain incubation period. When more than 40% of cells within 6 h to 12 h reach the other side of the filter and form colonies in an invasion assay like in Example 8 or Example 9, the naturally occurring cancer cell is defined as invasive. The control cells instead form only up to 5% colonies in the same time frame and are defined as non-invasive.

“Adhesiveness” as used herein is the ability of a cell to reattach after they have been removed from the matrix on which it had been grown, resuspended as a solution of single cells (not in direct contact with other cells of the solution), and replated on a matrix to which adhesion is possible. A cell is defined as adhesive if in an assay as described in Example 11 or Example 12, more than 40% of the cells adhere within a time of 30-120 min. Instead, only up to 5% of the control cells adhere within the same time frame.

Metastatic potential as used herein is the ability of a tumor cell to form a new tumor at a site distant from the primary tumor of which the tumor cell was derived (a metastase). Metastatic potential can be measured by injecting, e.g. 1×10⁶ , cells into the lateral tail vein of athymic nude mice and determining the number of tumor nodules in the lung, e.g. 2 months post injection, e.g. as described in the section “Tumor cell injections” on page 2346 of Huang et al (1996) Oncogene 13, 2339-2347, or the sections “Animals and production of tumors” and “Histochemical analysis for calcified matrix” on page 1882 of Radinsky et al. (1994) Oncogene 9: 1877-1883. A cell line to produce more than 3, preferably more than 8, more preferably more than 20 tumor nodules in the lung in this assay is considered metastatic.

Therapeutically effective amounts are amounts which eliminate or reduce the patient's tumor burden, or which prevent, delay or inhibit metastasis. The dosage will depend on many parameters, including the nature of the tumor, patient history, patient condition, the possible co-use of cytotoxic agents, and methods of administration. Methods of administration include injection (e.g., parenteral, subcutaneous, intravenous, intraperitoreal, etc), for which the molecule inhibiting neuropilin-1 function is provided in a nontoxic pharmaceutically acceptable carrier. In general, suitable carriers and diluents are selected so as not to significantly impair biological activity of the binding agent (e.g., binding specificity, affinity or stability), such as water, saline, Ringer's solution, dextrose solution, 5% human serum albumin, fixed oils, ethyloleate, or liposomes.). Acceptable carriers can include biocompatible, inert or bio-absorbable salts, buffering agents, oligo- or polysaccharides, polymers, viscoelastic compound such as hyaluronic acid, viscosity-improving agents, preservatives, and the like. In addition, the pharmaceutical composition or formulation may also include other carriers, adjuvants, or nontoxic, non-therapeutic, non-immunogenic stabilizers and the like. Typical dosages may range from about 0.01 to about 20 mg/kg, or more particularly from about 1 to about 10 mg/kg.

Therapeutic methods employing molecules inhibiting neuropilin-1 function may be combined with chemotherapy, surgery, and radiation therapy, depending on type of the tumor, patient condition, other health issues, and a variety of factors. The molecules inhibiting neuropilin-1 function may also be used as the single effective medicament of a therapeutic composition.

A “molecule inhibiting neuropilin-1 function”, is a molecule resulting in inhibition of the biological activity of neuropilin-1. This inhibition of the biological activity of neuropilin-1 can be assessed by measuring one or more indicators of neuropilin-1′s biological activity, such as neuropilin-1 dependent angiogenesis, neuropilin-1 dependent invasiveness or neuropilin-1 dependent adhesion. These indicators of neuropilin-1's biological activity can be assessed by one or more of several in vitro or in vivo assays (see, Examples 8 or 9 and Examples 11 or 12, or Example 16). Preferably, the ability of a molecule to inhibit neuropilin-1 activity is assessed by inhibition of neuropilin-1-induced invasiveness or adhesion of invasive human sarcoma cells, particularly the cells used in Examples 8 and 9 or Examples 11 and 12 or 19. Preferably, the ability of a molecule to inhibit neuropilin-1 activity is assessed by inhibition of neuropilin-1-induced angiogenesis of HUVEC cells, particularly as described in Example 16.

A “molecule inhibiting neuropilin-1 function” of the invention is not a molecule which is a general inhibitor of protein function, like a protease, like a denaturing agent, e.g. urea or guanidinium hydrochloride, like heavy metal atoms or like small molecules (e.g. aldehydes or isocyanates) reacting covalently and non-specifically with biomolecules (lipids, proteins, sugars). A molecule inhibiting neuropilin-1 function is characterized by its ability to inhibit neuropilin-1 function at a concentration at which it does not inhibit the function of the insulin receptor (e.g. as determined in an anti-Phosphotyrosine Western Blot Assay, see e.g. B. Cariou et al. (2002) J Biol Chem., 277, 4845-52) and the Acetylcholin receptor (e.g. as determined by measuring the Ca influx, see M. Montiel et al. (2001) Biochem Pharmacol. 63, 337-42.) and the B-CAM cell surface glycoprotein (e.g. by determining binding of hemoglobin A red blood cells (AA RBCs) to immobilized laminin as described in the section “Flow chamber assays” on page 2551 of Udani et al. (1998) J. Clin. Invest. 101, 2550-2558). Only a molecule inhibiting neuropilin-1 function but at the same concentration not significantly affecting the function of the other three receptors mentioned is a molecule inhibiting neuropilin-1 function as used in this patent. Inhibition is understood to be at least a 15%, preferably a 20%, more preferably a 25%, 30%, 40%, 50% or even a 60% decrease in function, as defined by neuropilin-1 function in an invasion and/or adhesion assay as mentioned above, when compared to a negative control with the same experimental conditions, but without the molecule of the invention. A molecule is defined as not significantly affecting the function of the other three receptors if the decrease in function affected by the molecule of the invention is less than 10%, more preferably less than 5%.

Additionally, in the case of a molecule of the invention which inhibits gene expression of neuropilin-1, such a molecule decreases neuropilin-1 expression by more than 50%, preferably by more than 80%, still more preferably by more than 90%, most preferably by more than 95% when measured in a quantitative western blot normalized to the level of beta tubulin present, when present in an experiment at a concentration of 10 nM to 100 μM, preferably at around 1 μM, in which the amount of neuropilin-1 is compared between two otherwise identical samples, wherein in one sample the molecule of the invention was allowed to inhibit neuropilin-1 expression. In the same experiment the molecule of the invention does not decrease the amount of the beta tubulin present per cell by more than 20%, and said molecule does not decrease the relative level of the insulin receptor and the B-CAM cell surface glycoprotein by more than 20%.

Additionally, in the case of a polypeptide of the invention, particularly an antibody or antibody fragment of the invention, the polypeptide of the invention is considered to inhibit the biological function of neuropilin-1 if it reduces the invasiveness and/or adhesiveness of naturally invasive cancer cells in an experiment as in Example 8 or 9 or Example 11 or 12 by more than 15%, preferably more than 20%, more preferably a 25%, 30%, 40%, 50% or even a 60% decrease in neuropilin-1 function as defined above, when said antibody fragment is present at a concentration of 1 nM to 50 μM, preferably around 20 μM.

Additionally, in the case of a small chemical compound of the invention, said compound is considered to inhibit the biological function of neuropilin-1 if it reduces the invasiveness and/or adhesiveness of naturally invasive cancer cells in an experiment as in Example 8 or 9 and Example 11 or 12 by more than 15%, preferably more than 20%, more preferably a 25%, 30%, 40%, 50% or even a 60% decrease in neuropilin-1 function as defined above, when present at a concentration of 10 nM to 100 μM, preferably at around 1 μM, while not affecting cell morphology, cell cycle progression (determined by analyzing the DNA content of a cell population by propidium iodide staining and FACS analysis), and not increasing the percentage of the cells of the culture that show signs of apoptosis (determined by measuring the percentage of cells showing DNA fragmentation, e.g. by a so called tunnel-assay). A small chemical compound as used in this invention is a molecule with a molecular weight between 50 Da and 10000 Da, preferably between 100 Da and 4000 Da, more preferably between 150 Da and 2000 Da, or a physiologically acceptable salt thereof.

“Identifying” as used herein means the identification of a biomolecule having desired properties from a mixture of biomolecules comprising related but non-identical biomolecules with slightly different properties.

A “ligand” as used herein is a molecule displayable by an amplifiable ligand-displaying unit. A ligand is that part of an ALDU through which the ALDU can bind to a target. Preferably it is a polypeptide as defined above, an RNA-oligonucleotide or a DNA-oligonucleotide an oligonucleotide comprising more than 20 base units but less than 10,000, preferably less than 1,000 base units. A ligand can bind to an extracellular region of an antigen. This binding may have specificity in the sense that the ligand binds to one antigen with high affinity but to a moderately related antigen with lower, for example 10- or 50- or 200-fold lower affinity. Moderately related antigens are antigens with up to 30% amino acid identity in the extracellular regions.

A ligand “binding specifically to a neuropilin-1” as mentioned herein can be a ligand which binds to neuropilin-1 under the buffer conditions given in Examples 2 and 3. The dissociation constant between the ligand and neuropilin-1 can be measured, e.g. by use of the so-called BIACORE System (see, for example, Fivash et al. Curr Opin Biotechnol. (1998) 9, 97-101) and “binding specifically” can then be understood to mean that the dissociation constant between the ligand and neuropilin-1 is lower than 10 μM, preferably lower than 1 μM, more preferably lower than 500, 400, 300, 200, 100, 50, 20 nM, most preferably from 0.1 nM to 20 nM if measured under standard conditions, for example at 20° C., ambient pressure and in a suitable buffer, e.g. 20 mM Tris, 100 mM NaCl, 0.1 mM EDTA at an overall pH of 7.0. Further, this molecule does not bind to neuropilin-2, which is another member of the neuropilin family and shares a 47% homology to neuropilin-1. Thus, the dissociation constant between the ligand and neuropilin-2 is higher than 100 preferably higher than 1 mM, or is alternatively at least 50-fold, preferably at least 200-fold, 1000-fold or 5000-fold worse (higher) than the dissociation constant between ligand and neuropilin-1.

The term “at least one” as used here means “one and more than one”, particularly one, two, three, four and five.

The present invention relates to polypeptides, antibodies, antibody fragments or single chain antibodies, which can specifically bind to the extracellular region of neuropilin-1 and can inhibit neuropilin-1 function in angiogenesis, invasion and/or metastasis. Such polypeptides, antibodies, antibody fragments or single chain antibodies are in the context of the present invention generally understood or summarized under the term “neuropilin binder (NPB)”. Those NPBs comprise a sequence selected from the group consisting of SEQ ID NO. 1 and SEQ ID NO. 2 or SEQ ID No. 5 to SEQ ID No 38.

In a preferred embodiment the polypeptide of the invention is an antibody fragment, in particular a scFv, dsFv, Fab′, Fab, F(ab′)2, Fv, single domain antibody or diabody, more particularly a scFv, dsFv, Fv, single domain antibody or diabody, still more particularly a scFv, single domain antibody or diabody and even more preferably a scFv.

In another preferred embodiment the polypeptide of the invention is an antibody, in one preferred embodiment an antibody derived from a scFv antibody fragment, in another preferred embodiment a polyclonal or monoclonal antibody, particularly a human monoclonal antibody.

Anti-human neuropilin-1 binding antibodies may be selected from modified immunoglobulin, for example chemically or recombinantly produced antibodies or humanized antibodies, site directed mutagenized antibodies, that exhibit substantial amino acid sequence identity in their CDR regions, particularly in their CDR3 region, to the corresponding antibody fragments of the invention and retain substantially the same affinity for neuropilin-1 binding as the corresponding antibody fragments.

In another preferred embodiment the polypeptide of the invention is a human antibody selected from the group consisting of IgA, IgD, IgE, IgG, and IgM, in particular IgG and IgM, more particularly IgG1, IgG2a, IgG2b, IgG3, IgG4.

In another preferred embodiment the CRD3 region of the antibody or the antibody fragment is identical to one of the CDR3 regions shown in FIG. 10.

In another preferred embodiment of the invention a polypeptide of the invention, to particularly an antibody fragment or an antibody of the invention is labeled with a detectable label. Particularly, examples for detectable labels are radioisotopes, chromophores, fluorophores, enzymes or radioisotopes. The detectable label can, for example, be selected from this group.

In another embodiment, the polypeptide of the invention can be covalently or non-covalently conjugated and/or coupled to or with, respectively, another protein, a solid matrix (e.g. like a bead), with itself to form multimers, a cytotoxic agent further enhancing the toxicity to targeted cells, a cytostatic agent, a prodrug, or an effector molecule, which is able to modify the cell expressing neuropilin-1 or to recruit immune cells. All these conjugates are “bioconjugates” of the invention.

A list of cytotoxic agents include, but is not limited to, daunorubicin, taxol, adriamycin, methotrexate, 5 FU, vinblastin, actinomycin D, etoposide, cisplatin, doxorubicin, genistein, andribosome inhibitors (e.g., trichosantin), or various bacterialtoxins (e.g., Pseudomonas exotoxin; Staphylococcus aureus protein A).

Bioconjugates comprising the polypeptides of the invention, particularly the antibody fragment or antibody of the invention together with said cytotoxic moieties are made using a variety of bifunctional protein coupling agents. Some examples of such reagents are N-succinimidyl 3-(2-pyridyldithio)-propionate (SPDP), bi-functional derivatives of imidoesters such a dimethyl adipimidate HCl, active esters such as disuccinimidyl suberate, aldehydes such as glutaraldehyde, bisazido compounds such as bis-(R-azidobenzoyl)hexanediamine, bisdiazonium derivatives such as bis-(R-diazoniumbenzoyl)ethylenediamine, diisocyanates such as tolylene 2,6-diisocyanate, and bis-activated fluorine compounds such as 1,5-difluoro-2,4-dinitrobenzene. Methods useful for the production of bioconjugates are described in detail in March's Advanced Organic Chemistry: Reactions, Mechanisms and Structure, 5th Edition, Wiley-Interscience; or Bioconjugate Techniques, Ed. Greg Hermanson, Academic Press.

The expression of a metastasis-associated neuropilin-1 antigen can be detected by using a bioconjugate or a,polypeptide of the invention, particularly an antibody or an antibody fragment of the invention. A sample is taken from the subject, e.g., a biopsy specimen taken from tissue suspected of having a metastatic tumor. Generally, the sample is treated before an assay is performed. Assays, which can be employed include ELISA, RIA, EIA, Western Blot analysis, immunohistological staining and the like. Depending upon the assay used, the antigens or the antibodies can be labeled by an enzyme, a fluorophore or a radioisotope. (See, e.g., Coligan et al. (1994) Current Protocols in Immunology, John Wiley & Sons Inc., New York, New York; and Frye et al. (1987) Oncogene 4, 1153-1157.)

Therefore, one embodiment of the invention relates to the use of at least one polypeptide of the invention and/or at least one labeled polypeptide of the invention and/or at least one bioconjugate of the invention for the detection of neuropilin-1.

For example one polypeptide of the invention or one labeled polypeptide of the invention or one bioconjugate of the invention can be used for the detection of neuropilin-1, or one labeled polypeptide together with one bioconjugate or two or three polypeptides or two labeled polypeptides can be used.

In another embodiment, the present invention encompasses a diagnostic kit. Such a kit comprises at least one bioconjugate and/or at least one labeled polypeptide of the invention and/or at least one polypeptide of the invention, particularly an antibody fragment or an antibody of the invention, or a labeled version of these, and consists additionally of the reagents and materials necessary to carry out a standard competition or sandwich assay. Said diagnostic kit may be used for the determination of the invasive potential of biological samples, in particular of certain cancer cell types. A kit will further typically comprise a container.

By using the polypeptide of the invention, particularly the antibody fragment or antibody of the invention, it is further possible to produce anti-idiotypic antibodies, which can be used to screen antibodies to identify whether the antibody has the same binding specificity as a human monoclonal antibody of the invention and can also be used for active immunization (Herlyn et al. (1986) Science, 232, 100). Such anti-idiotypic antibodies can be produced using well-known hybridoma techniques (Kohler et al. (1975) Nature, 256:495). An anti-idiotypic antibody is an antibody, which recognizes unique determinants present on the antibody of interest. These determinants are located in the hypervariable region of the antibody. It is this region, which binds to a given epitope and, thus, is responsible for the specificity of the antibody. An anti-idiotypic antibody can be prepared by immunizing an animal with the polypeptide, particularly the antibody fragment or antibody, of interest. The immunized animal will recognize and respond to the idiotypic determinants of the immunizing antibody and produce an antibody to these idiotypic determinants. By using anti-idiotypic antibodies, it is possible to identify other hybridomas expressing monoclonal antibodies having the same epitopic specificity.

It is also possible to use the anti-idiotype technology to produce monoclonal antibodies, which mimic an epitope. For example, an anti-idiotypic monoclonal antibody made to a first monoclonal antibody will leave a binding domain in the hypervariable region, which is the “image” of the epitope bound by the first antibody. Thus, the anti-idiotypic monoclonal antibody can be used for immunization, since the anti-idiotype monoclonal antibody binding domain effectively acts as an antigen.

In another embodiment the modified polypeptide or the bioconjugate of the invention, binds to human neuropilin-1 and reduces the invasiveness and/or adhesiveness of invasive human sarcoma cells by 15-70%, or preferably by 15-30%, 20-40% or even at least 50%, when tested in an invasion/or adhesion assay (see Example 8 or 9 and Example 11 or 12).

In another embodiment, the antibody fragment of the invention specifically recognizes one or more epitopes of neuropilin-1, or epitopes of conserved variants of neuropilin-1, or peptide fragments of the neuropilin-1.

In another embodiment, the invention relates to the use of a molecule of the invention, particularly selected from the group consisting of a small chemical compound of the invention, a molecule inhibiting gene expression of neuropilin-1, a bioconjugate or a polypeptide of the invention, more particularly an antibody fragment or an antibody of the invention as a medicament.

In another embodiment, the present invention relates to a pharmaceutical composition comprising effective amounts of at least one, particularly one, molecule of the invention, particularly at least a bioconjugate of the invention or at least one of a molecule inhibiting gene expression of neuropilin-1, more particularly wherein the molecule is an antibody fragment or antibody of the invention, in combination with a pharmaceutically acceptable carrier and/or a diluent.

The pharmaceutical composition can be used for the treatment of conditions related to the over-expression or ectopic expression of human neuropilin-1, especially the treatment of metastatic tumors, especially of metastatic tumors derived from the group selected of cancer cell-types of page 12.

The pharmaceutical composition of the invention can further be used for the treatment tumors or metastases whereby the comprised NPBs inhibit angiogenesis and the outgrowing of blood vessels, which provide the tumor with nutrition's, and thereby famish the tumor.

In another embodiment of the invention, pharmaceutical compositions are provided comprising a pharmaceutically acceptable carrier and a therapeutically effective amount of at least one molecule inhibiting neuropilin-1 function can, particularly be a molecule which can bind to the extracellular region of neuropilin-1, more particularly wherein the molecule is a small chemical compound or a modified polypeptide or the bioconjugate of the invention, still more preferably wherein the molecule is a modified scFv of the invention or a modified antibody derived from such a scFv of the invention.

In another embodiment, the invention relates to a method of treating or preventing tumors or metastasis in a patient comprising the administration of a molecule inhibiting neuropilin-1 function, in a pharmaceutical acceptable composition in an amount effective to treat, i.e. inhibit, delay or prevent, metastasis of neuropilin-1 mediated invasion and/or adhesion. The molecule inhibiting neuropilin-1 function can particularly be a molecule which can bind to the extracellular region of neuropilin-1, which can be identified be the method of identifying a ligand binding specifically to the extracellular region of neuropilin-1 described below, more particularly wherein the molecule is a small chemical compound or an antibody or an antibody fragment or a modified polypeptide of the invention or a bioconjugate of the invention, still more preferably wherein the molecule is a modified scFv of the invention or a modified antibody derived from such a scFv of the invention. This method of treating or preventing metastasis can be effective to reduce or inhibit the invasion and/or adhesion of cancer cells derived from mesodermal cells, in particular cancer cells derived from sarcomas, in particular cancer cells derived from sarcomas of the bone and/or soft tissue. The cancer cells can be selected from the group of cancer cells described on page 12.

The present invention further relates to a. method to produce the polypeptide of the invention by recombinant techniques. These techniques are well known in the art (Skerra et al. (1993), Curr. Opin. Immunol. 5, 256-62; Chadd et al. (2001), Curr. Opin. Biotechnol. 12, 188-94).

For example, nucleic acid sequences encoding a polypeptide of the invention, particularly an antibody fragment or an antibody (e.g., a gene encoding an antibody fragment of FIG. 10 or an antibody thereof) can be isolated and cloned into one or more polynucleotide expression vectors, and the vector can be transformed into a suitable host cell line for expression of a recombinant polypeptide of the invention. Expression of the gene encoding the polypeptide of the invention provides for increased yield of the polypeptide, and also allows for routine modification of the polypeptide by introducing amino acid substitutions, deletions, additions and other modifications, for example humanizing modifications (Rapley (1995) Mol. Biotechnol. 3: 139-154) in both the variable and constant regions of the antibody fragment or of the antibody of the invention without critical loss of binding specificity or neuropilin-1 blocking function (Skerra et al. (1993) Curr. Opin. Immunol. 5, 256-262).

The present invention therefore relates to an above mentioned isolated nucleic acid molecule encoding any one of the polypeptides of the invention, particularly an antibody fragment of the invention, more particularly a scFv, dsFv, Fv, single domain antibody or diabody of the invention, still more particularly a scFv, single domain antibody, diabody of the invention or an antibody derived from such a scFv of the invention, and even more preferably a scFv of the invention or an antibody derived from such a scFv of the invention.

In a preferred embodiment the present invention relates to a nucleic acid molecule encoding a NBP comprising a sequence selected from the group consisting of SEQ ID NO. 1 and SEQ ID NO. 2, or SEQ ID No. 5 to SEQ ID No. 38.

The present invention further relates to a vector comprising a nucleic acid of the invention. Particularly the vector is a plasmid, a phagemid, or a cosmid.

For example, the nucleic acid molecule of the invention can be cloned in a suitable fashion into procaryotic or eucaryotic expression vectors (Sambrook et al., “Molecular cloning: a laboratory manual” Second edition, Cold Spring Harbor Laboratory Press (1989)). Such expression vectors comprise at least one promotor, at least one signal for translation initiation, at least one nucleic acid sequence of the invention and—in the case of procaryotic expression vectors—a signal for translation termination, while in the case of eucaryotic expression vectors preferably additional signals for transcriptional termination and for polyadenylation.

Examples for prokaryotic expression vectors are, for expression in Escherichia coli, e.g. expression vectors based on promotors recognized by T7 RNA polymerase, as described in U.S. Pat. No. 4,952,496, for eucaryotic expression vectors for expression in Saccharomuces serevisiae, e.g., the vectors p426Met25 or 526GAL1 (Mummberg et al. (1994) Nucl. Acids Res., 22, 5767-5768), for the expression in insect cells, e.g., Baculovirus-vectors as e.g. described in EP-B1-0 127 839 or EP-B1-0 549 721, and for the expression in mammalian cells, e.g., the vectors Rc/CMV and Rc/RSV or SV40-vectors, which are commonly known and commercially available.

The molecular biological methods for the production of these expression vectors, as well as the methods of transfecting host cells and culturing such transfected host cells as well as the conditions for producing and obtaining the polypeptides of the invention from said transformed host cells are well known to the skilled person.

The present invention further relates to a host cell comprising a nucleic acid of the invention and/or a vector of the invention, particularly wherein the host cell is a microorganism like yeast or other fungi, like Escherichia coli, Bacillus subtilis or other bacteria. The host cell can also be a cell of higher eucaryotic origin, like an insect cell, preferably a virus infected insect cell, more preferably a baculovirus infected insect cell, or like a mammalian cell like HeLa, COS, MDCK 293-EBNA1, NSO or a hybridoma cell.

The present invention relates further to a method for the production of a polypeptide of the invention, particularly an antibody fragment of the invention, comprising culturing a microorganism transformed with a recombinant vector comprising DNA encoding a polypeptide of the invention, particularly an antibody fragment of the invention, and recovering said polypeptide of the invention, particularly an antibody fragment of the invention or a fusion protein containing it, from the medium.

The present invention shows that blocking neuropilin-1 function may inhibit invasiveness and/or adhesiveness of certain cancer cells derived from group selected of cancer cell-types of page 12, and particularly inhibits invasiveness and or adhesiveness of cancer cells, e.g., derived from human sarcoma cells, lymphoma cells and mesothelial neoplasms, more particularly human sarcoma cells.

One embodiment of the invention is therefore the use of at least one, particularly one, molecule inhibiting neuropilin-1 function in the manufacture of a medicament for the treatment or prevention of invasion and/or metastasis of naturally occurring cancer cells, wherein invasiveness and/or metastatic potential of said cancer cells depends on neuropilin-1 function, particularly of such tumor cells which are derived from mesodermal cells or cancer cells derived from the group selected of the cancer cell-types of page 12.

In another preferred embodiment the molecule inhibiting neuropilin-1 inhibits the function of expressed neuropilin-1. Expressed neuropilin-1 is to be understood in this context as neuropilin-1 protein already present on naturally occurring cancer cells before any kind of treatment is initiated.

These molecules are particularly molecules, which bind to the extracellular region of neuropilin-1.

The extracellular segment of neuropilin-1 is defined as that part of the neuropilin-1 protein outside of the cellular membrane, consisting of five domains a1, a2, b1, b2 and c. Domains, which are specifically involved in cell adhesion are b1 and b2. It should be appreciated that neuropilin-1 is a glycoprotein, so not only the mentioned amino acids, but also the sugar modifications on them are considered as being part of the extracellular segment of neuropilin-1.

More particularly this molecule is selected from the group consisting of a small chemical compound, an antibody against neuropilin-1, an antibody fragment against neuropilin-1, a polypeptide of the invention, an anti-idiotypic antibody of the invention and/or a bioconjugate of the invention, especially wherein the molecule is a polypeptide and/or a bioconjugate of the invention.

In another preferred embodiment the naturally occurring cancer, which depend on neuropilin-1 function for angiogenesis, invasiveness, adhesiveness and/or metastatic potential can be any cancer wherein the cancer cells are cells derived from the group of neoplasms consisting of adenocarcinomas, sarcomas, morbus hodgkin, non-hodgkin lymphoma of high and low malignancy, multiple myeloma, malignant tumors of the brain, head and neck tumors, carcinoma of the thyroid, mesothelioma, leukemia, carcinoma of the esophagus, stomach and pancreas carcinoma, primary carcinomas of the liver, carcinoma of bilary duct and bladder, colorectal carcinoma, basel cell carcinoma, malignant melanoma, osteosarcoma, malignant gliomas, Ewing carcinoma, soft tissue sarcoma, Kaposi sarcoma, nephroblastoma, neuroblastoma, carcinoma of the kidney, testicular carcinoma, prostate carcinoma, carcinoma of the urinary bladder, malignant tumors of the ovaries, carcinoma of the endometrium of the cervix, tumors of the adrenal gland, particularly from the group of neoplasms consisting of malignant fibrous histiocytoma, liposarcoma, fibrosarcoma, synovial sarcoma, osteosarcoma (parosteal osteosarcoma, periosteal osteosarcoma, small-cell osteosarcoma), chondrosarcoma, Ewing's sarcoma, giant-cell tumor of bone, osteogenic sarcoma, leiomyosarcoma, rhabdomyosarcoma, mesothelioma, lymphangiosarcoma, myxosarcoma, endotheliosarcoma, chordoma, Kaposi's sarcoma and lymphangioendotheliosarcoma.

The invention further pertains to the neuropilin-1 antigen as a druggable target. Another aspect of the present invention pertains to antibody fragments that bind to human neuropilin-1 with high neutralizing capacity.

In another embodiment of the invention, at least one polypeptide of the invention and/or a bioconjugate of the invention are used for identifying additional molecules that specifically bind human neuropilin-1, particularly in screening assays. These methods entail contacting a reference anti-neuropilin-1 antibody fragment with a target species comprising the neuropilin-1 domain in the presence of a putative competitor test-binding agent. This step of contacting is conducted under conditions suitable for complex formation between the reference antibody fragment and the target species in the absence of the test-binding agent. Complex formation between the reference antibody fragment and the target species in the presence of the test-binding agent is detected as an indicator of specific binding activity of the test-binding agent to neuropilin-1. This screening method is useful for high throughput screening of, e.g., other antibody libraries or antibody fragment libraries, antisense oligonucleotide libraries or peptide and small molecule libraries to identify and characterize additional “molecules binding specifically to neuropilin-1”. Competition is determined by an assay in which the antibody fragment, or other binding agent under test substantially inhibits specific binding of the reference antibody fragment to the target species containing the neuropilin-1 domain. This can be determined for example by measuring binding of the reference antibody fragment to a target species comprising neuropilin-1 domain in the presence and absence of a putative competitor, i.e. a “molecule binding specifically to neuropilin-1” under conditions suitable for complex formation. Numerous types of competitive binding assays are known and routinely practicable within the invention, as described for example in U.S. Pat. No. 4,376,110. Typically, such assays involve the use of a target species containing the neuropilin-1 domain (e.g., purified neuropilin-1 or a cell line expressing the neuropilin-1 antigen), an unlabeled “molecule binding specifically to neuropilin-1”, and a labeled reference antibody fragment or other binding agent. Competitive inhibition is measured by determining the amount of label bound to the target species in the presence of the “molecule binding specifically to neuropilin-1”. Usually the “molecule binding specifically to neuropilin-1” is present in excess. “Molecules binding specifically to neuropilin-1” identified by these competition assays (“competitive binding agents”) include antibodies, antibody fragments, peptides, antisense oligonucleotides, small molecules and other binding agents that bind to an epitope or binding site bound by the reference antibody fragment, as well as a “molecule binding specifically to neuropilin-1” that bind to an epitope or binding site sufficiently proximal to an epitope bound by the reference antibody fragment. Preferably, competitive binding agents of the invention will, when present in excess, inhibit specific binding of a reference antibody fragment to a selected target species by at least 10%, preferably by at least 25%, more preferably by at least 50%, and even more preferably by at least 75%-90% or greater.

In addition to a polypeptide of the invention, particularly a modified antibody fragment or a modified antibody of the invention, natural or artificial ligands, peptides, anti-sense, or other small molecules capable of specifically targeting human neuropilin-1 may be employed. Drugs can be designed to bind or otherwise interact and inhibit human neuropilin-1 based upon the present invention. In this regard, rational drug design techniques such as X-ray crystallography, computer-aided (or assisted) molecular modeling (CAMM), quantitative or qualitative structure-activity relationship (QSAR), and similar technologies can be utilized to focus drug discovery efforts. Rational design allows prediction of molecules, which can interact with proteins or specific parts thereof. Such molecule structures can be synthesized chemically and/or expressed in biological systems. Small to molecules may be produced by synthesizing organic compounds according to methods that are well known in the art. A plurality of peptides, semi-peptidic compounds or non-peptidic, and organic compounds may be synthesized and then screened in order to find compounds, which bind to neuropilin-1 with high neutralizing capacity. Particularly compounds that inhibit neuropilin-1 related invasion. See generally Scott and Smith, “Searching for Peptide Ligands with an Epitope Library”, Science (1990), 249, 386-90 and Devlin et al., “Random Peptide Libraries: A Source of Specific Protein Binding Molecules”, Science, (1990), 249, 40407.

The present invention also provides methods of using the modified antibody or modified antibody fragments to inhibit human neuropilin-1 activity or to detect human neuropilin-1 in cancer cells, preferably sarcoma cells, either in vitro or in vivo. In a preferred embodiment, treating cells expressing the antigen with one or more modified antibody fragments causes or leads to a reduction or inhibition of the invasive or adhesive abilities of human cancer cells, particularly of human sarcoma cells.

The migration of tumor cells into tissue is an important step in metastasis. The processes of adhesion and invasion can be studied in the transendothelial model (See, Woodward et al. (2002) Invest Ophthalmol Vis Sci 43, 1708-14 and Vachula et al. (1992) Invasion Metastasis 12, 66-81). The transendothelial model provides a useful ex vivo, e.g. an in vitro, system for the investigations of cellular interactions during the invasion process.

The present invention therefore further provides an ex vivo, e.g. an in vitro method to determine the dependency of the invasiveness of a naturally occurring invasive cancer cell on the functionality of neuropilin-1. This method comprises the steps of:
a. contacting the cells with a molecule inhibiting neuropilin-1 function;
b. contacting the cancer cell with a gel-like matrix, under conditions suitable for the growth of said cancer cells; and
c. determining the migration of said cancer cells through the gel-forming matrix.

The term “gel-like matrix” as used herein is understood to be a semi-solid substance with a water content of at least 90%, which allows cultivation of cancer cells in contact with the matrix and allows migration of invasive cancer cells through a slab of said “gel-like matrix” of 0.1 mm to 1 mm, preferably 0.3 mm thickness, but not migration of non-invasive cells. Examples for such a “gel-like matrix” are substances resembling the extracellular matrix in protein and carbohydrate composition, particularly the commercially available “Matrigel”. Particularly the “gel-like matrix” comprises one of the proteins selected from the group consisting of the proteins collagen type IV, fibronectin and laminin. More particularly the gel-like matrix comprises the proteins collagen type IV, fibronectin and laminin. More preferably, the gel-like matrix comprises the proteins collagen type IV, laminin, entactin, nidogen and heparan sulfate proteoglycans or collagen type IV, fibronectin, laminin, nidogen, entactin, and vitronectin.

In a preferred embodiment the interfering molecule of step a) is a polypeptide that specifically binds to an extracellular epitope of neuropilin-1, particularly a modified polypeptide of the invention, more particularly a modified antibody or a modified antibody fragment of the invention, still more particularly a modified antibody fragment, even more particularly a modified scFv, dsFv, Fv, single domain antibody or diabody, especially a modified scFv, single domain antibody or diabody and even more preferably a modified scFv.

The present invention therefore further provides an ex vivo, e.g. an in vitro method to determine the dependency of the adhesiveness of a naturally occurring invasive cancer cell on the functionality of neuropilin-1. This method comprises the steps of:
a. contacting the cells with a molecule inhibiting neuropilin-1 function;
b. contacting the cancer cell with a layer of ECM proteins, under conditions suitable for the growth of said cancer cells; and
c. determining the adhesion of said cancer cells to the layer of ECM proteins.

The term “layer of ECM proteins” as used herein is understood to be a semi-dry layer of a protein solution, which allows cultivation of cancer cells in contact with the layer and allows attachment of invasive cancer cells to said layer. The thickness of said “layer of ECM proteins” is between 0.1 mm to 1 mm, preferably 0.3 mm thick. Examples for “ECM proteins are substances of the extracellular matrix. Particularly “ECM proteins are selected from the group consisting of the proteins collagens, entactin, nidogen, vitronectin, fibronectin and laminins. More particularly ECM proteins are selected from collagen S type I, collagen type IV, fibronectin and laminin.

In a preferred embodiment the interfering molecule of step a) is a polypeptide that specifically binds to an extracellular epitope ofneuropilin-1, particularly a modified polypeptide of the invention, more particularly a modified antibody or a modified antibody fragment of the invention, still more particularly a modified antibody fragment, even more particularly a modified scFv, dsFv, Fv, single domain antibody or diabody, especially a modified scFv, single domain antibody or diabody and even more preferably a modified scFv.

The present invention further provides a method of identifying a ligand, binding specifically to the extracellular region of NP-1, by screening a naïve or immune library, preferably a phage display library, wherein said ligand is capable of interfering with the functionality of neuropilin-1, specifically wherein said ligand is capable of inhibiting angiogenesis, tube formation of endothelial cells, and/or invasion or adhesion of tumor cells. Said method comprises the steps of
a) contacting a phage library of ligands with cancer cells or endothelial cells;
b) isolating said cells;
c) removing phages bound unspecifically to said cells, e.g. by washing said cells with a buffered detergent solution, under conditions where said cells do not lyse;
d) eluting phages bound to said cells; and
e) determining the identity of the ligand represented by said eluted phages
f) testing the ligand in biochemical or biological assays on it capability to interfere with NP-1 function.

The identity of phages representing the ligand obtained with step e) can be determined by, e.g., sequencing the DNA encoding the ligand in case the ligand is an antibody or antibody fragment, or, in the case of a commercial ligand library with gridded or numbered phages, by determining the grid position or the number of the phage. The grid position or the number then can reveal the identity of the ligand represented by the phage.

After step d) the pool of phages is enriched in phages binding to NP-1. Those phages binding to NP-1 can finally be identified by numerous methods known in the art. Phages can be separated to form individual clones and the clones of the phages can be probed with labeled NP-1 protein, or a labeled part of the NP-1 protein, e.g. an at least seven amino acid long peptide of the extracellular region of NP-1. Clones binding to such a probe are identified as NP-1-binders. Phages can also be affinity purified on purified NP-1 protein or on recombinant NP-1.

Alternatively, in case the ligand is e.g. an antibody or antibody fragment, the open reading frame encoding the antibody or antibody fragment can be recloned from the whole enriched pool into an expression vector, the antibody or antibody fragment can then be expressed in clones of another host cell, and the clone of the host cell carrying the expression vector comprising a nucleic acid encoding for the antibody or antibody fragment binding specifically to NP-1 can be identified, e.g. by the method described above for the identification of relevant phage clones, by the method of Examples 2 and 3, or by affinity purification on recombinant NP-1.

A particular advantage of this method is that the ligand specific for the accessible part of the extracellular region of NP-1 is obtained, since the initial selection step is performed on intact cells, which present the accessible part of the extracellular region of NP-1 for binding of the phages.

In another preferred embodiment of the invention the method comprises an additional step of screening on recombinant NP-1 protein. This method is described more specifically in Example 3.2.

The method comprises instead of step e) the further steps of:
e) contacting isolated phages with recombinant NP-1;
f) washing said NP-1 with a buffered detergent and/or high salt solution; and
g) eluting phages bound to NP-1; and
h) determining the identity of the ligand represented by said eluted phages.

In certain embodiments, the ligand expressed on the phages comprise an antibody or an antibody fragment selected from the group consisting of scFv, dsFv, Fab′, Fab, F(ab′)₂, Fv, single domain antibodies (DABs) and diabodies, more particularly selected from the group consisting of scFv, dsFv, Fv, single domain antibody or diabody, still more particularly selected from the group consisting of scFv, single domain antibody or diabody and even more preferably a scFv.

The “detergent” used in steps c) and f) is a detergent solution, preferably buffered, and can be Tween in a concentration of 0.001-0.5%, particularly 0.01-0.1%. “High salt” in step f) is a high salt solution, preferably buffered, and has an ionic strength of 10 mM-1M, particularly 20-500 mM, more particularly 50-350 mM, even more preferably 80-250 mM. Typical useful anions are, for example, chloride, citrate, phosphate, hydrogen phosphate or borate. Typical useful cations are, for example, sodium, potassium, lithium, calcium or magnesium.

The buffered solution in the above paragraph typically has a pH of 7-8. For example, DMEM or PBS, particularly with 1-20%, more particularly 5-15%, even more preferably about 10% FCS, can be used as buffers.

Isolation of cells with phages bound to them is effected by gentle centrifugation at g values from 200 to 300 for 3 to 20 minutes, particularly 5 to 10 minutes. Elution of bound phages, both to cells and to immobilized NP-1, is effected by a wash with 2-100 mM, particularly 4-50 mM, more particularly 5-20 mM, even more preferably around 10 mM glycine at a pH of from 0 to 2.5, particularly from 1 to 2.5, more particularly from 1.5 to 2.5.

The NP-1 interfering or inhibiting functionality of these ligands, particularly of these antibodies or antibody fragments may be assayed as described above, in vivo or in a cell culture experiment. Cell culture assays include assays that determine the inhibitory effect of the ligands of this invention in an invasion or adhesion assay as described in Examples 8 and 9 or 19 or Examples 11 and 12, or in a tube formation assay with endothelial cells as described in Example 16. Results of the different assays are provided in the Figures.

The method advantageously combines a screening step based on binding to the surface of a cell with a screening step based on a functional assay, which identifies ligands having the capability to interfere or inhibit NP-1 function.

In a preferred embodiment the ligand is capable of inhibiting a biological function of neuropilin-1. For example, the biological function of a neuropilin-1 can be invasion, adhesion, or angiogenesis. Adhesion and invasion have been explained as adhesiveness and invasiveness before.

An invasion assay measures the invasiveness of a cell. An invasion assay is, e.g., the assay performed in Example 8 and 9 of this application.

An adhesion assay measures the adhesiveness of a target, e.g. a cell, to something else, e.g. another cell, a virus, or a complex biological mixture like, e.g., a population of vehicles derived from a cell or, e.g., the basal lamina. An example of an adhesion assay is given in Example 11 and 12 of the present application.

“Angiogenesis” is the process where cells induce blood vessel formation in their proximity. Angiogenesis can accompany the growth of malignant tissue and can therefore be a property of malignant cells. That is to say that malignant cells can have the property of inducing angiogenesis. An angiogenesis assay measures the ability of a cell to induce blood vessel formation, a process, which usually accompanies the growth of malignant tissue. An angiogenesis assay is disclosed in Kanda et al (2002) J. Natl. Cancer Inst. 94, 1311-9. An example of an angiogenesis assay, a so-called tube formation assay is given in Example 16 of the present application.

In another preferred embodiment the method of identifying a ligand binding specifically to the extracellular region of neuropilin-1 also comprises the step of incorporating the ligand identified into a therapeutic, prophylactic or diagnostic composition. This can, e.g., be done by mixing-the identified ligand with a pharmaceutically acceptable carrier known in the art, wherein the ligand is present in an amount, which is therapeutically effective.

In another preferred embodiment the invention relates to a method for the production of a pharmaceutical composition, comprising the method of identifying a ligand binding specifically to neuropilin-1 and further the step of mixing the identified ligand, or a modified or a labeled version thereof, with a pharmaceutical acceptable carrier known in the art. A modified ligand is a ligand with a covalent modification. Examples of modified ligands are labeled ligands.

The invention further provides an advantageous combination of methods, which allow a functional increase of activity of the previously identified ligand. For this the method for identifying a ligand, which interferes with NP-1 functionality, is combined with the method of CALI (Chromophore-Assisted Laser Inactivation).

The principle of CALI is based on the local initiation of a photochemical reaction that leads to the generation of short-lived reactive species, which in turn selectively modify the target molecule and cause its functional inactivation. Highly specific but non-inhibitory ligands (e.g. antibodies, antibody fragments, small molecules) are labeled with a suitable chromophore (e.g. malachite green, fluorescein, methylene blue, eosin). After complex formation between the target molecule (e.g. proteins) and the ligand, the complex is irradiated with laser light of an appropriate wavelength to excite the chromophore. The excitation triggers a photochemical reaction that initiates the generation of short-lived reactive species (e.g. hydroxyl radicals or highly reactive oxygen species). These reactive species modify the protein within a small radius around their site of generation. The distance that a reactive species can travel is very short due to its short lifetime. Therefore, the modifications of amino acid residues within the protein occur in close proximity to the binding site of the ligand. The damaging effect is restricted to a radius of 15-40 Å which is well below the average distance of two proteins within a cell, which is at about 80 Å, (assuming an average cytosolic protein concentration of 300 mg/ml and an average protein size of 50 kDa) ensuring a high spatial resolution of the process. This principleis shown in FIG. 13. As an example, a modified antibody against a given protein often can selectively inhibit the function of that particular protein after CALI, even if this antibody did not show an inhibiting function without CALI. In cases where the binding site of the ligand is close or within an important functional domain of the protein, these induced modifications lead to permanent inactivation of the protein. The functional inactivation of the protein is measured in an appropriate readout assay and evaluated in the context of disease relevant physiological functions like cell invasion, cell adhesion, cell signaling or apoptosis.

Inactivation of proteins with CALI was shown to be very specific to the respective protein. Linden et al showed that β-galactosidase could be efficiently inactivated with a malachite-green labeled anti-β-galactosidase antibody even in the presence of alkaline phosphatase in the same solution. β-galactosidase was inactivated by 95% after 10 min of laser irradiation whereas alkaline was not effected at all (Linden et al. (1992) Biophys. J. 61, 956-962). Jay also demonstrated that a dye-labeled antibody bound to a single epitope of a protein was sufficient to inactivate acetylcholinesterase (Jay (1988) Proc. Natl. Acad. Sci. USA, 85, 5454-58).

Henning et al. described that CALI was successfully used against a diverse array of proteins (Henning et al. Innovations in Pharmaceutical Technology (2002) 62-72). These protein include membrane proteins (eg. α-, β-, ε-chains of T cell receptor, β1 integrins, ephrin A5 or FAS receptor), signal transduction molecules (eg. calicineurin, cyclophillin A or PKC), cytoskeletal proteins (eg. actin, ezrin or kinesin) or transcription factors. Henning et al. further described that CALI can be used for identification of novel proteins as drug targets and at the same time the elucidation of their function in the biological context of interest (Henning et al. (2002) Current Drug Discovery May, 17-19).

Several application examples of CALI show that CALI is able to convert specific but non-inhibitory ligands into blocking reagents. Therefore, these ligands can be used to modulate the action of inhibitory ligands. CALI can also be used to further enhance the inhibitory effect of a ligand that has already an inhibitory effect by itself. Example 9 and Example 12 are examples where CALI is integrated in an invasion and an adhesion assay, respectively.

In another embodiment of the invention the ligand can be modified.

The chemical modification of the ligand can be the addition of chromophores. A chromophore is that part of a moledule that possesses high optical activity due to mobile electrons that interact with light. Some examples of chromophores are, e.g., fluorescein derivatives, rhodamine derivatives, coumarin derivatives, porphyrin derivatives, phthalocyanine derivatives, naphthalocyanines derivatives eosin derivatives, triphenylmethane derivatives or acridine derivatives. A list of chromophores useful for chemically modifying biomolecules is disclosed in The Sigma Aldrich Handbooks of Stains and Dyes, Ed., F. J. Green (1990) ISBN No. 0-941633-22-5.

The method of identifying a ligand according to the invention may further comprise at least one additional step of testing the ligand, e.g. wherein the testing is based on biochemical or biological properties of the ligand. Such an additional testing step may comprise a method selected from the group consisting of flow cytometry, ELISA, immunoprecipitation, binding assays, immunohistochemical experiments, affinity studies, immunoblots and protein arrays. Biochemical properties can be determined by the size, the shape, the density, the charge density, the hydrophobicity, or the binding specificity of the ligand. The biochemical properties of the ligand form the basis of the applicability of the above-mentioned methods.

In another preferred embodiment of the invention, the method of identifying a ligand of the invention can further comprise a substractive selection step. A substractive selection step is a step, which removes ligands with an undesired property. For example a substractive selection step can be affected by removing the ligands capable of binding to a control cell, if the property of binding to a control cell is undesired. By way of example, if one wants to select for ligands specific for cancer cells one could first select those ligands which bind to cancer cells, elute the bound ligands, e.g. phages, and then remove those ligands which are capable of binding to non-cancer-cells, by for example contacting the pool of eluted ligands with non-cancer-cells and removing those ligands bound to the non-cancer-cells. The ligands remaining in the supernatant are then ligands specific for cancer cells and can then be used in functional screening assays according to the method of identifying a ligand of the invention.

The neuropilin-1 inhibitory activity of the identified NP-1 binding ligands or antibody fragments, particularly of scFvs may be assayed as described above, in vivo or in a cell culture experiment.

Cell culture assays include assays that determine the inhibitory effect of the antibody fragments of this invention in an angiogenesis, invasion or adhesion assay as described in Examples 16, 8 or 9 and Examples 11 or 12. Results of the angiogenesis assay are provided in FIG. 14. Results of the invasion assay are provided in FIG. 2. Results of the adhesion assay are provided in FIGS. 3 and 4.

In this part of the invention particularly neuropilin-1-binders (NPB) are described, which modulate neuropilin-1 (NP-1) functions particularly, but not limited to the context of angiogenesis. As described above neuropilin-1 (NP-1) is known as a coactivator of VEGF, an essential and strong activator for the induction of blood vessel growth. It was therefore an object of the invention to provide NPBs that interfere with the NP-1/VEGF interaction and, thereby, modulate or inhibit NP-1 function.

The NPBs of the invention are preferably polypeptides, antibodies, antibody fragments or bioconjugates, more preferably single chain antibodies (scFv) or corresponding IgGs, which were generated by cloning the scFv-specific DNA sequence into an IgG expressing vector. According to a further embodiment these NPBs were labeled with detectable labels as described above.

The NPBs of the invention preferably bind to an extracellular epitope of the NP-1. Cross-reactivity to NP-2 or other members of the NP family is not excluded.

The NPBs of the invention bind to various extracellular epitopes of NP-1. In one embodiment the NPBs bind to the epitope where to also VEGF binds and which is accordingly involved in the induction of angiogenesis. Such NPBs have the capacity to block and inhibit VEGF/NP-1 interaction. Consequently, they further have the capability to interfere or inhibit the VEGF-dependent induction of angiogenesis. Such NPBs are particularly useful as medicament and for the treatment of angiogenesis.

It is further known that the various splice variants of VEGF also bind to slightly different epitopes of NP-1. The invention also provides NPBs that bind to these different epitopes. Such NPBs have the capacity to block and inhibit the interaction of the splice variants of VEGF and NP-1. Thus, also they have the capability to interfere or inhibit the VEGF-dependent induction of angiogenesis. Such NPBs are particularly useful as medicament and for the treatment of angiogenesis.

Unexpected, surprising and new was the fact that the invention also identified and characterized NPBs that did not bind to any of the known VEGF-binding epitopes, but still modified or inhibited angiogenesis. Such NPBs modulating and inhibiting the NP-1 function, but not interfering with the VEGF/NP-1 interaction are provided in a preferred embodiment. They have the capability to interfere or inhibit the induction of angiogenesis and are particularly useful as medicament and for the treatment of angiogenesis.

For characterization of the NPB's of the invention, said NPBs were tested e.g. in a so-called tube formation assay. In such an assay endothelial cells, which normally express NP-1, are incubated with the various NPBs and than the effect of such binding on tube formation or cell aggregation is analyzed (see Example 16, and FIGS. 14 and 15).

In parallel these NPBs were tested in a biochemical VEGF/NP-1 interaction assay (see Example 17). For this a competitive ELISA was used where the binding between a VEGF and the various NPBs was qualitatively and quantitatively measured.

The NPBs identified in the different assays are provided by further preferred embodiments. Accordingly, one embodiment provides NPBs, particularly scFv and corresponding IgG, which inhibit tube formation. Particularly preferred according to this embodiment are the scFvs 7, 8, 11, 12, 13, 15, 18, 21, 23, 25, 26, 27, 28, 29, 31, 33 and 36 as well as the corresponding IgGs.

It is particularly interesting and surprising, that although all NPBs of this embodiment show a strongly inhibiting effect on tube formation, not all bind to or cross-react with the epitopes, which is bound by VEGF. This is explicitly shown in the competitive ELISA of Example 17, wherein the capability of the different NPBs of the invention is tested to inhibit the interaction and/or binding of NP-1 and VEGF.

Several NPBs clearly interfere and prevent the interaction of NP-1/VEGF. Most of these binders show a strong inhibiting effect on tube formation. A preferred example of this embodiment is scFv8 or the corresponding IgG. According to a further embodiment such NBPs are highly useful for medical treatment, particularly the treatment or prevention of tumor-related and metastasis-related angiogenesis. Consequently, a further embodiment provides compositions and preparations comprising such NPBs as medicaments or for the treatment of NP-1-dependent angiogenesis, most preferably of tumor related angiogenesis.

According to another embodiment the invention provides NPBs that do not interfere or prevent the interaction of NP-1/VEGF, but still inhibit tube formation. A preferred example of this embodiment is scFv13 or the corresponding IgG. Furthermore preferred are the NPBs that bind to the same epitope as scFv13. Preferred are also NPBs that do not bind to the same epitope as VEGF₁₆₅. The assay as described in Example 18 is particularly useful to identify further NPBs, which bind to the same epitope as scFv 13 or another epitope as VEGF₁₆₅.

The following examples, including experiments conducted and results achieved, are provided for illustrative purposes only and are not to be construed as limiting upon the present invention.

### DESCRIPTION OF THE DRAWINGS

FIG. 1 shows the invasion of stained HT1080 cells through an 8 μm Matrigel-coated filter. Fluorescence was quantified after six hours incubation at 37° C. Data presented are the mean of n=3 wells+/−SD.

FIG. 2 shows the inhibitory effect of scFv1 on the invasion of HT1080 cells. Invasion was determined in a chemotaxis assay with a Matrigel coated cell migration chamber. The invasion was determined after laser irradiation (with CALI, gray bars) and without laser irradiation (without CALI, dark gray bars). The invasion of HT1080 cells in the absence of any inhibitory molecule was used as a control (left two bars).

FIG. 2a shows the influence of VEGF on the invasiveness of HT1080 cells. Invasion was determined in a chemotaxis assay with a Matrigel coated cell migration chamber. The invasion of cells could only be stimulated with FCS (first and fourth bar from the left). No significant increase of invasion was seen when the cells were pre-incubated with recombinant VEGF (second bar from the left. The addition of anti-human VEGF antibody (aVEGF) had no influence on this result (third bar from the left). BSA as a chemo-attractant did not stimulate invasion (fifth bar from the left). Addition of recombinant VEGF to the chemo-attractant BSA did not stimulate the invasion (sixth bar from the left). The addition of anti-human VEGF antibody (aVEGF) had also no effect on this result (seventh bar from the left).

FIG. 3 shows the inhibitory effect of scFv1 and scFv2 on the adhesion of HT1080 cells. Adhesion to collagen S type I was determined after laser irradiation (with CALI, gray bars) and without laser irradiation (without CALI, dark gray bars). The adhesion of HT1080 cells to collagen S type I in the absence of any inhibitory molecule was used as a control (left two bars).

FIG. 4 shows the inhibitory effect of scFv1 and scFv2 on the adhesion of HT1080 cells to different matrix proteins. The adhesion of HT1080 cells to the different matrix proteins in the absence of any inhibitory molecule was used as a control (left block of bars).

FIG. 5 shows results of FACS analysis of scFv1 and scFv2 with different cells lines (bold line) and HS-27 cells (dotted line) as control.

FIG. 6 shows the result of the immunoprecipitation experiments. scFv1 was tested on HT1080 cells and compared to HS-27 cells as a control. The immuno-complexes were separated by SDS-PAGE and silver stained. The band at 130 kDa was identified as neuropilin-1.

FIGS. 7a and 7b show the vector map and sequence of the scFv display vector pXP10.

FIGS. 8a and 8b show the vector map and sequence of the scFv expression vector pXP14.

FIG. 9 shows the sequences for the construction primers for a mouse library.

FIG. 10 shows the amino acid sequences of scFvl to scFv36. In each sequence the CDR3 is marked by underlining. The corresponding SEQ ID's are indicated.

FIG. 11 shows the nucleotide sequences coding for the polypeptides scFv1 to scFv36.

FIG. 12 shows a MALDI-MS spectrum of the peptide mixture obtained from the band with an approximate size of 130 kDa immunoprecipitated with scFv 1. Two trypsin auto digestion peaks, indicated as T, were used for internal calibration. A total of 17 peaks, marked with asterisks, matched neuropilin-1 (SwissProt, 014786), with a mass deviation of less than 10 ppm. The matched peptides cover 22% (206/923 residues) of the protein.

FIG. 13 shows the principle of Chromophore-Assisted Laser Inactivation (CALI).

FIG. 14 shows the results of the tube formation assay. The degree of tube formation was determined via light microscopy and was quantified based on the ability of cells to build closed polygons, the number and area of the polygons, the number of branch points and the ability of the cells to form closed tubes e.g. connections between branch points. Inhibitory effects were quantified by comparison with a negative control and were scaled between 0 and 3 (0-1=no inhibition and 2-3=strong inhibition effect). 14a) Control experiment without addition of an inhibitory antibody, inhibition of tube formation=0, closed polygons are formed, high numbers of branch points, branch points are connected and no significant cell aggregation. 14b) After incubation with 50 μl/ml of scFv25*, inhibition of tube formation=3.0, no closed polygons are formed, very low number of branch points and branch points are not connected. The symbol “*” indicates that the respective scFv—as identified by its number as shown in FIG. 10—was cloned into an IgG1 format before performing the assay. 14c) After incubation with 100 μl/ml of a control antibody (anti-α2 integrin antibody), inhibition of tube formation=3.0, no closed polygons are formed, no branch points visible.

FIG. 15 shows—presented as a table—all results of the tube formation assay. The degree of tube formation was determined via light microscopy and quantified as described in FIG. 14. The symbol “*” indicates that the respective scFv—as identified by its number as shown in FIG. 10—was cloned into an IgG1 format before performing the assay. The commercially available anti-NP-1 antibody did not show any significant inhibition of tube formation. “PBS 10%” indicates a control experiment, without the addition of an antibody.

FIG. 16 shows the inhibition of VEGF₁₆₅:NP-1 interaction tested in a competition ELISA. “*” indicates a p-value<0.01. Twelve scFv's of 28 were found to significantly inhibit the interaction between NP-1 and VEGF₁₆₅.

FIG. 17 shows the determination of epitopes for scFv's on cell surface NP-1. HT1080 cells were preincubated with a control scFv, different NP-1-binding scFv or VEGF as indicated in the legend and the binding of fluorescein labeled scFv was tested. The data show that scFv8 and scFv24 and VEGF have mutually overlapping epitopes, whereas scFv13 has an epitope distinct from scFv8,and scFv24 and VEGF.

FIG. 18 shows—presented as a table—results of the Transendothelial Invasion assay with HT1080 cells. scFv26, scFv27, scFv34 and scFv35 inhibited the invasion of TH1080 cells. The invasion is determined in % of inhibition of invaded cells. For inhibition values: “+” represents 1-10% inhibition.

FIG. 19 shows results of the migration assay with HUVEC cells. scFv8*, scFv25*, scFv26*, and scFv31* showed an statistically relevant inhibitory effect on the migration of HUVEC cells. The symbol “*” indicates that the respective scFv—as identified by its number as shown in FIG. 10—was cloned into an IgG1 format before performing the assay. The three bars to the left show the dependency of the migration of HUVEC cells on the concentration. of VEGF₁₆₅. Concentrations between 0-5 ng/ml were used. It can be seen that the migration of HUVEC cells increases with the increase of VEGF₁₆₅.

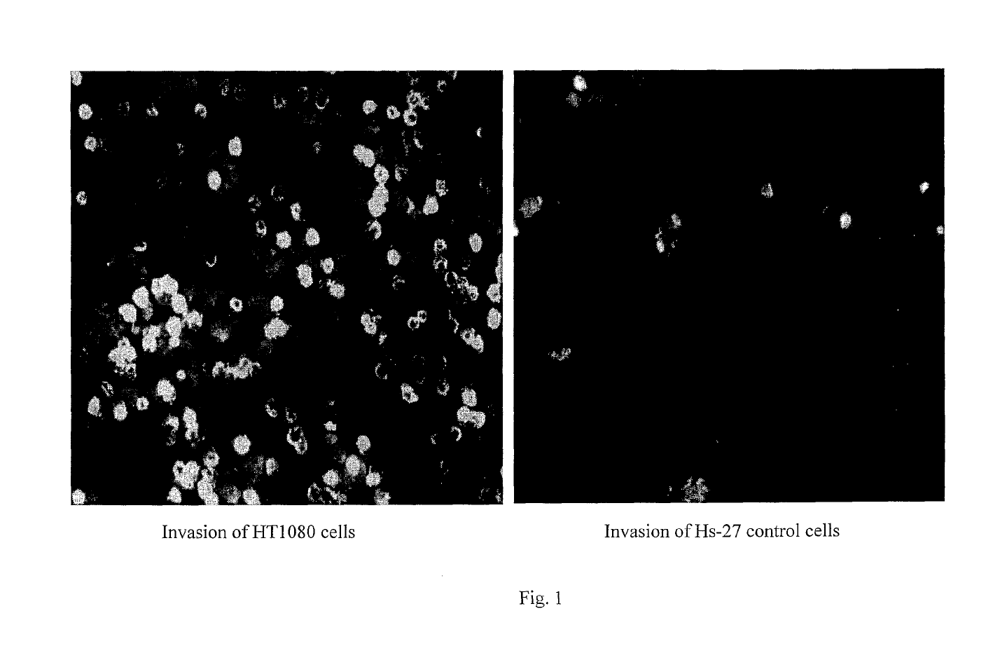

### EXAMPLES

### Example 1

### Construction of an Immune Library

Two BALB/c mice were each immunized intradermally with 2×10⁷ paraformaldehyde fixed HT1080 cells (human fibrosarcoma cell line; ATCC, CCL-121). Following the first immunization, the injections were repeated twice in a period of 39 days, the mice sacrificed and the spleens isolated and frozen in liquid nitrogen. The immunizations were performed by Charles River, Germany GmbH, Kii3legg.

Total RNA was isolated using the RNeasy Midi Kit (QIAGEN #75142) as described by the manufacturer using half of each spleen preparation. The RNA concentration and purity was determined by a denaturing formaldehyde gel and photometric measurement.

cDNA was synthesized using 8.9 μg of freshly prepared RNA and 10 pmol of a primer mix (IgGl-c, IgG2a-c, IgG2b-c, IgG3-c, VLL-c, VLK-c) using the Superscript™ II Kit (GibcoBRL Life Technologies #18064-014) These primers anneal to the RNA encoding the IgG heavy-chain genes and the light chain genes of the kappa and lambda family. VH genes were PCR amplified from 1 μl of cDNA using 36 individual combinations of 9 forward primers (MVH1, M+VH2, MVH3, MVH4, MVH5, MVH6, MVH7, MVH8, MVH9) and 4 backward primers (M−JH1, M−JH2, M−JH3, M−JH4) without restriction sites. VL genes were PCR amplified with one primer mix (M−VK1, M−VK2, M−VK3 M−VK4, M−VL1, M−JK1, M−JK2, M−JK3, M−JL1) without restriction sites. PCR products were gel-purified (QIAquick Gel Extraction Kit, #28706) and re-amplified using individual combinations of 9 forward primers (MVH1 SfiI, MVH2 Sfil, MVH3 SfiI, MVH4 SfiI, MVH5 SfiI, MVH6 SfiI, MVH7 SfiI, MVH8 SfiI, MVH9 SfiI) and 4 backward primers (M-JH1 SalI, M-JH2 SalI, M-JH3 SalI, M-JH4 SalI) with restriction sites for VH and one primer mix (M-VK1 ApaLI, M-VK2 ApaLI, M-VK3 ApaLI, M-VK4 ApaLI M-VL1 ApaLI, M-JK1 Nod, M-JK2 NotI, M-JK3 Notl, M-JL1 NotI) with restriction sites for VL. PCR products were gel-purified (QIAquick Gel Extraction Kit, #28706) and cloned into the phage display vector pXP10 using the restriction sites SfiIISalI for VH and ApaLI/NotI for VL. The ligation mix was transfected into E. coli TG-1 by electroporation resulting in a library size of 10⁷ independent clones.

### Example 2

### Selection and Screening of scFv (Selection on Fixed cells)

Single chain Fv were selected from a phage display library generated from mice immunized with fixed HT1080 cells. The library was generated using the phage display vector pXP 10.

HT1080 cells were harvested with 0.05% EDTA, fixed with paraformaldehyde, diluted to 1×10⁷cells/ml in PBS and immobilized onto wells of a 96 well UV cross-link plate (Corning Costar). The wells of the UV cross-link plate were blocked with 5% Skim Milk Powder (#70166, Fluka) in PBS (MPBS). 10¹² cfu (colony forming units) of phage library/10⁶ cells were pre-blocked for 1 hour at 25° C. with MPBS and subsequently incubated for 1.5 hour at room temperature (RT) with the cells. The wells of the UV cross-link plate were washed six times with PBS+0.05% Tween-20 followed by six washes with PBS. Bound phage were eluted by the addition of 10 mM Glycine pH 2.2, and neutralized with 1M Tris/HCl pH 7.4. Typically, between 10³ and 10⁶ cfu were eluted in the 1^{st} round of selection, thus the diversity of the enriched repertoire is decreased compared to the original repertoire. The eluate containing the enriched repertoire was amplified by infecting exponentially growing E. coli TG1. Phagemid containing E. coli were selected and propagated by overnight growth at 30° C. on LB agar plates supplemented with 100 μg/ml ampicillin and 1% glucose. Following this step, the enriched repertoire can either be amplified as a polyclonal pool and used for further rounds of selection in an iterative manner until convergence to desired properties is achieved or be spatially separated and screened on a single clone level. Phage to particles for the next round of selection were produced by super-infecting exponentially growing cultures of the previous round of selection with helper phage VCS-M13 (Stratagene, La Jolla, Calif.) and growing the cultures overnight at 20° C. in 2×TY supplemented with 100 μg/ml ampicillin and 50 μg/ml kanamycin. Selection ready phage were precipitated with 0.5 M NaCl/4% PEG-6000 from the cleared bacterial supernatant and re-suspended in PBS. One round of selection was performed followed by screening on a single clone level.

### Example 2.1

### Selection and Screening of scFv (Selection on Cells in Suspension)

Single chain Fv were selected from a large non-immune phage displayed repertoire of human origin containing 10¹¹ independent clones, provided by Cambridge Antibody Technology Ltd., Cambridge, UK.

For selection, HT1080 cells (human fibrosarcoma cell line; ATCC, CCL-121) were harvested with 0.05% EDTA and diluted to 1×10⁷cells/ml in DMEM+10% FCS. Two times 10¹² cfu of phage library/10⁷ cells were pre-blocked for 1 hour at 25° C. with DMEM+10% FCS and subsequently incubated with end-over-end rotation for 1.5 hour at 25° C. with the cells in Eppendorf tubes pre-blocked with DMEM+10% FCS. Three times 10⁷ cells were used for the first round of selection and 1×10⁷ cells were used for the 2^{nd} round of selection, respectively. The cells were washed by centrifugation at 220×g for five minutes, followed by removal of the supernatant and re-suspension in wash buffer. Five washes with DMEM+10% FCS+0.05% Tween-20 as wash buffer and five washes with DMEM+10% FCS as wash buffer were performed. Bound phages were eluted by the addition of 10 mM glycine pH 2.2, neutralized with 1M Tris/HCl pH 7.4. Typically, between 10³ and 10⁶ cfu were eluted in the 1^{st} round of selection, thus the diversity of the enriched repertoire is decreased compared to the original repertoire. The eluate containing the enriched repertoire was amplified by infecting exponentially growing E. coli TG1. Phagemid containing E. coli were selected and propagated by overnight growth at 30° C. on LB agar plates supplemented with 100 μg/ml ampicillin and 1% glucose. Following this step, the enriched repertoire can either be amplified as a polyclonal pool and used for further rounds of selection in an iterative manner even until convergence to desired properties is achieved or be spatially separated and screened for a desired function on a single clone level. Phage particles for the next round of selection were produced by super-infecting exponentially growing cultures of the previous round of selection with helper phage VCS-M13 (Stratagene, La Jolla, Calif.) and growing the cultures overnight at 20° C. in 2×TY supplemented with 100 μ/ml ampicillin and 50 μg/ml kanamycin. Selection ready phage were precipitated with 0.5 M NaCl/4% PEG-6000 from the cleared bacterial supernatant and re-suspended in PBS. In this example two rounds of selection were performed followed by screening on a single clone level.

### Example 3

### Selection and Screening of scFv (Screening on Fixed Cells)

For screening, the genes encoding the selected scFv, contained in the phage display vector, were re-cloned to the expression vector pXP14. This vector directs the expression of a scFv in fusion with a Streptag and E-tag and does not contain a filamentous phage gene-3. Expression vector containing E. coli TG1 from single colonies were grown in individual wells of a microtiter plate so that each well contains only one scFv clone. The bacteria were grown at 30° C. in 2× TY supplemented with 100 μg/ml ampicillin and 0.1% glucose in 96-well microtiter plates (#9297, TPP) until an OD₆₀₀ of 0.7. Expression was induced with IPTG at a final concentration of 0.5 mM and continued at 25° C. overnight. Single chain Fv containing cleared lysates were prepared by addition of hen-egg lysozyme (#L-6876, Sigma) to a final concentration of 50 μg/ml for 1 hour at 25° C. and centrifugation for 15 minutes at 3000×g. Prior to the screening ELISA, the cleared lysates were blocked by the addition of an equal volume of DMEM+10% FCS for 1 hour. For the screening ELISA, HT1080 cells were harvested with 0.05% EDTA, fixed with paraformaldehyde, diluted to 1×10⁷cells/ml in PBS and immobilized onto wells of a 96 well UV cross-link plate (Corning Costar). The wells of the UV cross-link plate were blocked with MPBS and the scFv containing blocked cleared lysates added for 1.5 hours at 25° C. The plates were washed 2× with PBS+0.1% Tween-20 and 1× with PBS, incubated with HRP conjugated α-E-tag (#27-9413-01, Pharmacia Biotech; diluted 1:5000 in MPBS with 0.1% Tween-20) for 1 hour, washed 3× with PBS+0.1% Tween-20 and 3× with PBS, developed with POD (#1 484 281, Roche) and signals read at 370 nm. Positive clones were retested against HT1080 cells and control human fibroblasts Hs-27 (ATCC CRL-1634) using the ELISA screening procedure described above.

In a typical screen, 2760 (30×92) clones were screened for binding to HT1080 cells with 5% positives defined as clones giving a background subtracted signal >0.1. 155 positive clones were retested for specific binding to HT1080 cells compared to the Hs-27 control cells with 28% positives defined as clones giving a background subtracted signal on HT1080 of twice the value of the signal on Hs-27 control cells. scFv1 was identified by applying this selection and screening method.

### Example 3.1

### Selection and Screening of scFv (Screening on Adherent Cells)

For screening, the genes encoding the selected scFv, contained in the phage display vector, were re-cloned to the expression vector pXP14. This vector directs the expression of a scFv in fusion with a Streptag and E-tag and does not contain a filamentous phage gene-3. Expression vector containing E. coli TG1 from single colonies were grown in individual wells of a microtiter plate so that each well contains only one scFv clone. The bacteria were grown at 30° C. in 2× TY supplemented with 100 μg/ml ampicillin and 0.1% glucose in 96-well microtiter plates (#9297, TPP) until an OD₆₀₀ of 0.7. Expression was induced with IPTG at a final concentration of 0.5 mM and continued at 25° C. overnight. Single chain Fv containing cleared lysates were prepared by addition of hen-egg lysozyme (#L-6876, Sigma) to a final concentration of 50 μg/ml for 1 hour at 25° C. and centrifugation for 15 minutes at 3000×g. Prior to the screening ELISA, the cleared lysates were blocked by the addition of an equal volume of DMEM+10% FCS for 1 hour. For the screening ELISA, HT1080 cells were seeded in a 96-well microtiter plate (#9296, TTP) at a density of 3×10⁴ cells/well in DMEM+10% FCS overnight at to 37° C. The wells were blocked with DMEM+10% FCS for 1 hour at 37° C. and the scFv containing blocked cleared lysates added for 1.5 hours at 25° C. The plates were washed 2× with PBS+0.1% Tween-20 and 1× with PBS, incubated with HRP conjugated α-E-tag (#27-9413-01, Pharmacia Biotech; diluted 1:5000 in 5% Skim Milk Powder (#70166, Fluka) in PBS with 0.1% Tween-20) for 1 hour, washed 3× with PBS+0.1% Tween-20 and 3× with PBS, developed with POD (#1 484 281, Roche) and signals read at 370 nm. Positive clones were retested against HT1080 cells and control human fibroblasts Hs-27 (ATCC CRL-1634) using the ELISA screening procedure described above.

In a typical screen, 1472 (16×92) clones were screened for binding to HT1080 cells with 16% positives defined as clones giving a background subtracted signal >0.1. 238 positive clones were retested for specific binding to HT1080 cells compared to the Hs-27 control cells with 28% positives defined as clones giving a background subtracted signal on HT1080 of twice the value of the signal on Hs-27 control cells. scFv2 was identified by applying this selection and screening method.

### Example 3.2

### Generation of Function Inhibiting Antibodies

Using a large naive human phage displayed antibody library provided by Cambridge Antibody Technology Ltd., Cambridge, UK, additional NP-1 antibodies were selected on recombinant NP-1. The selection was performed as described in (Vaughan, T. J et al., 1996 Nat Biotechnol. 14, 309). After two rounds of selection, individual clones were screened for their ability to specifically recognize recombinant NP-1 in ELISA and NP-1 as presented on the cell surface in cell ELISA and FACS.

### Example 4a

### Sequencing and Large Scale Expression

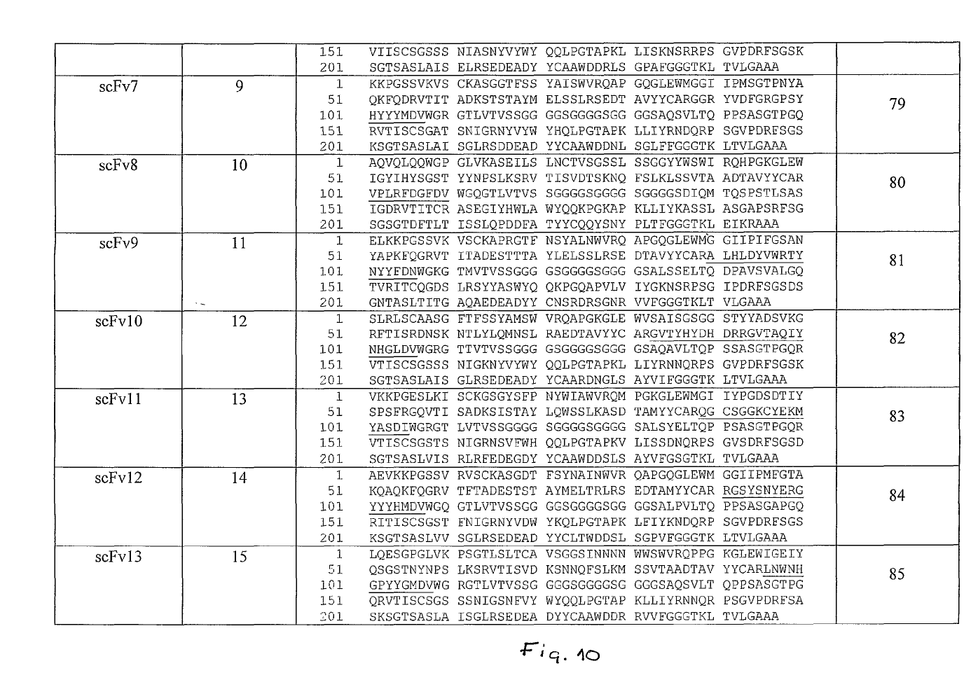

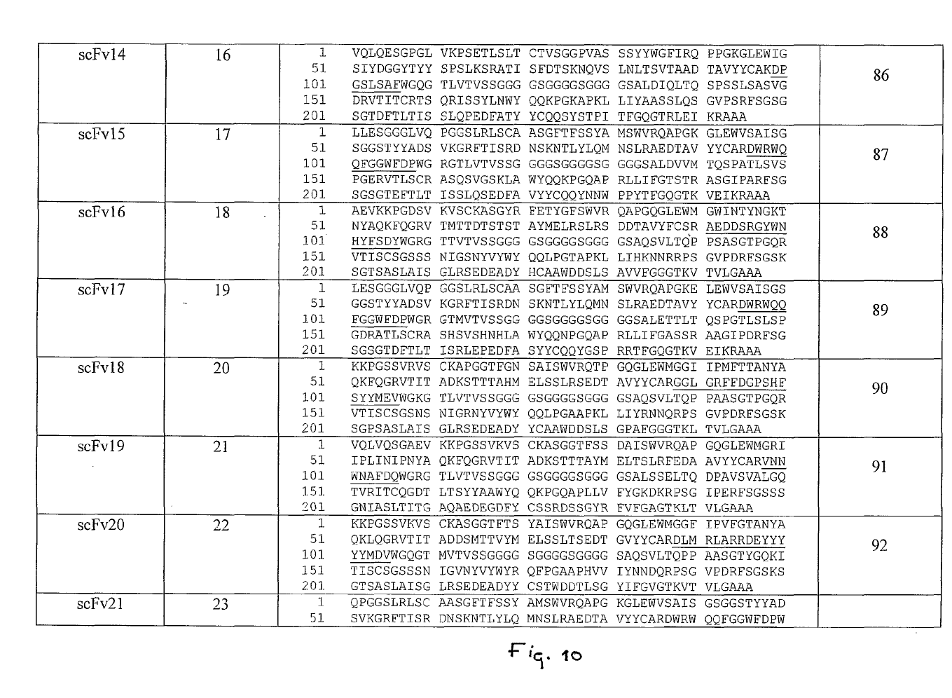

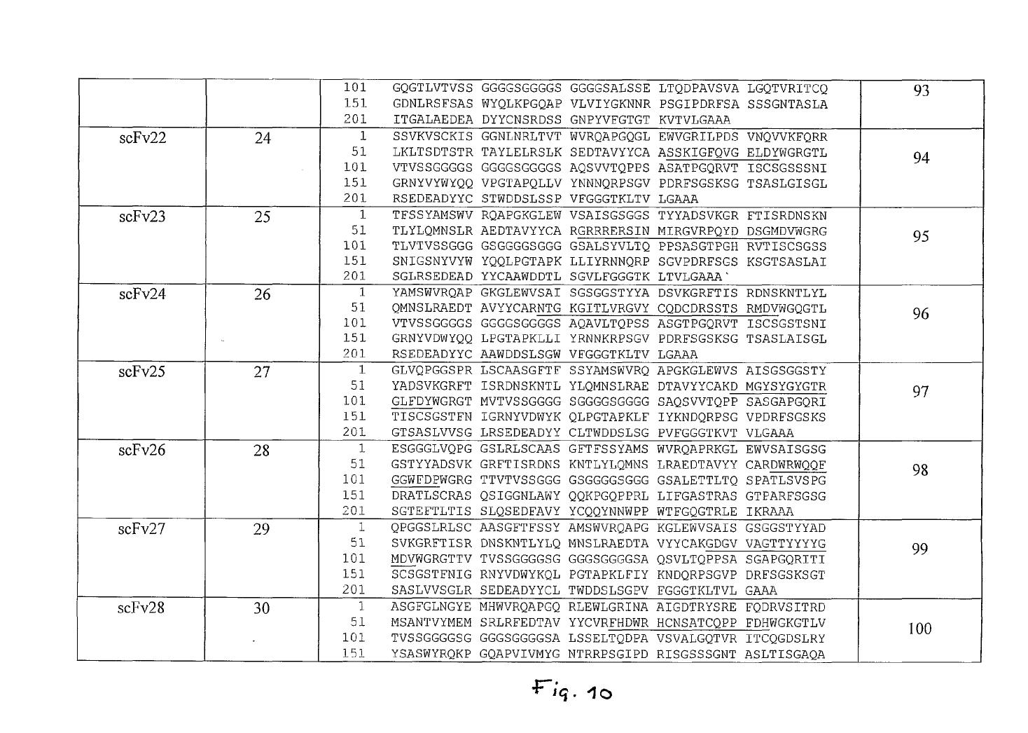

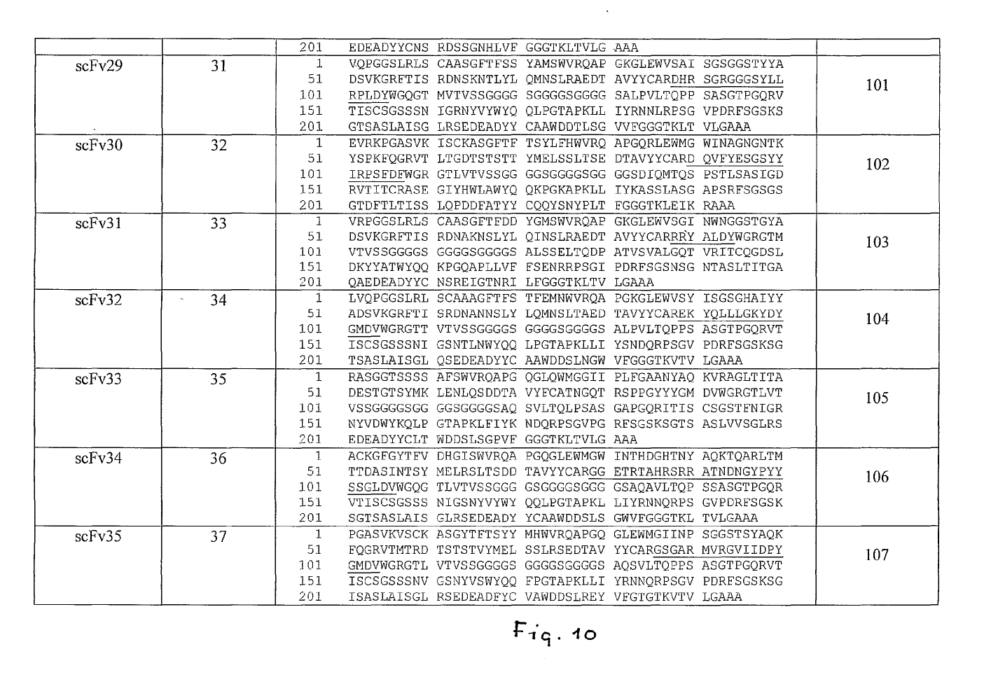

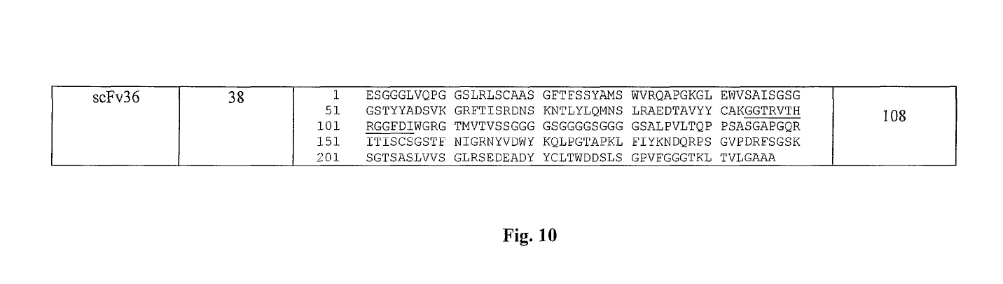

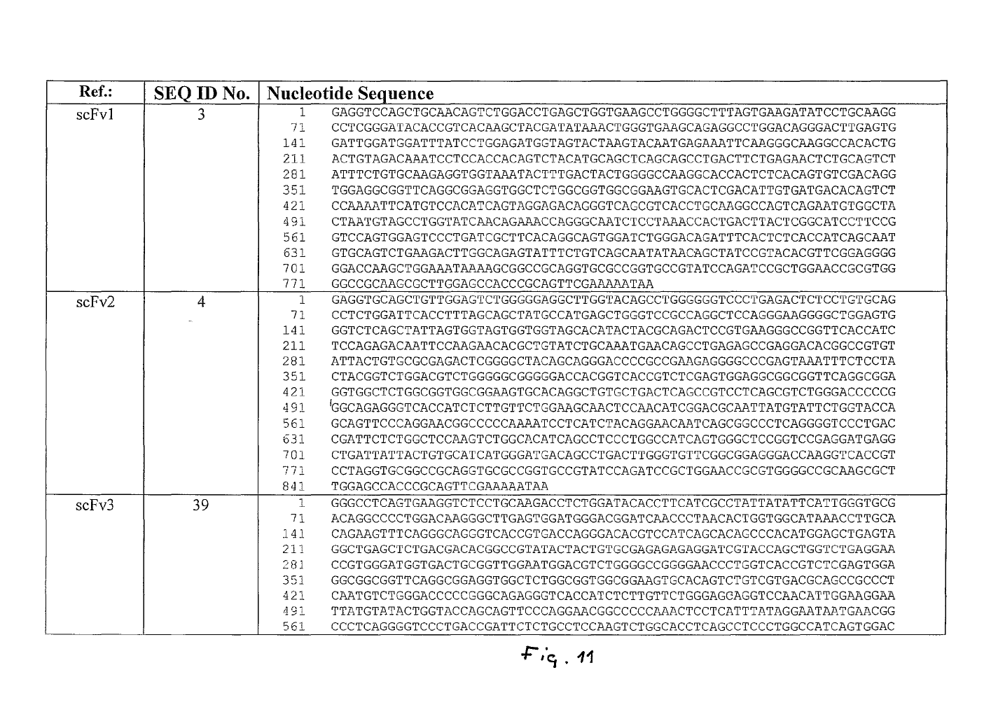

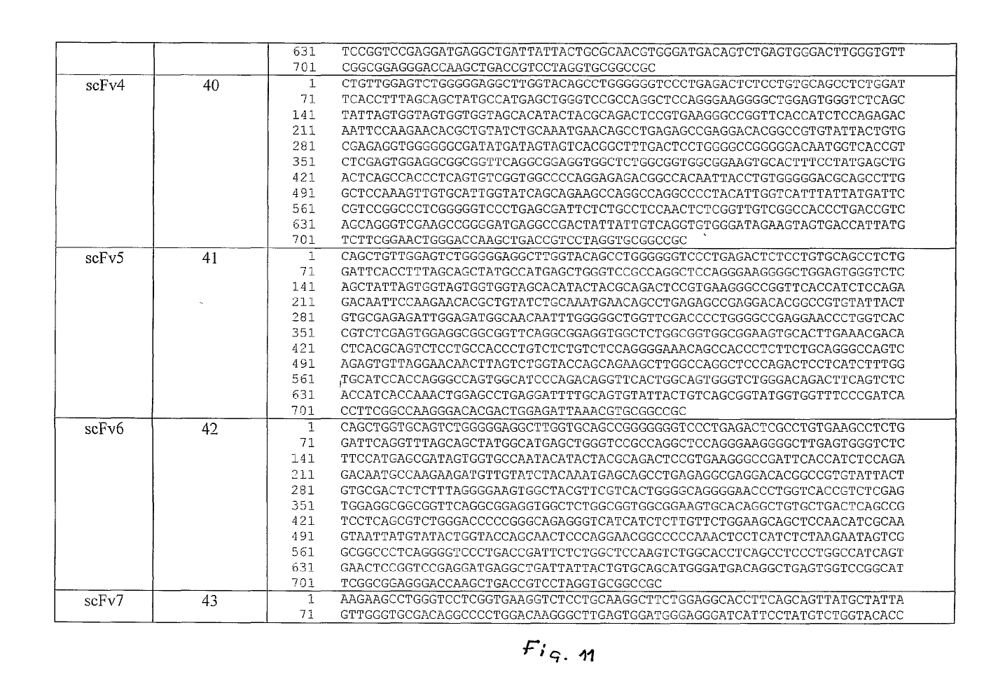

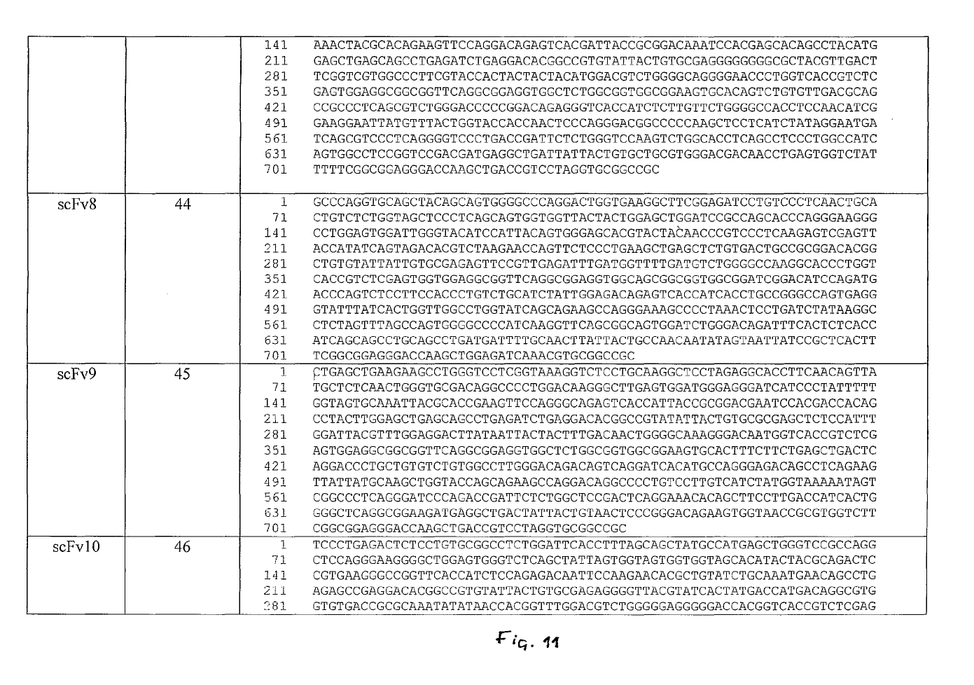

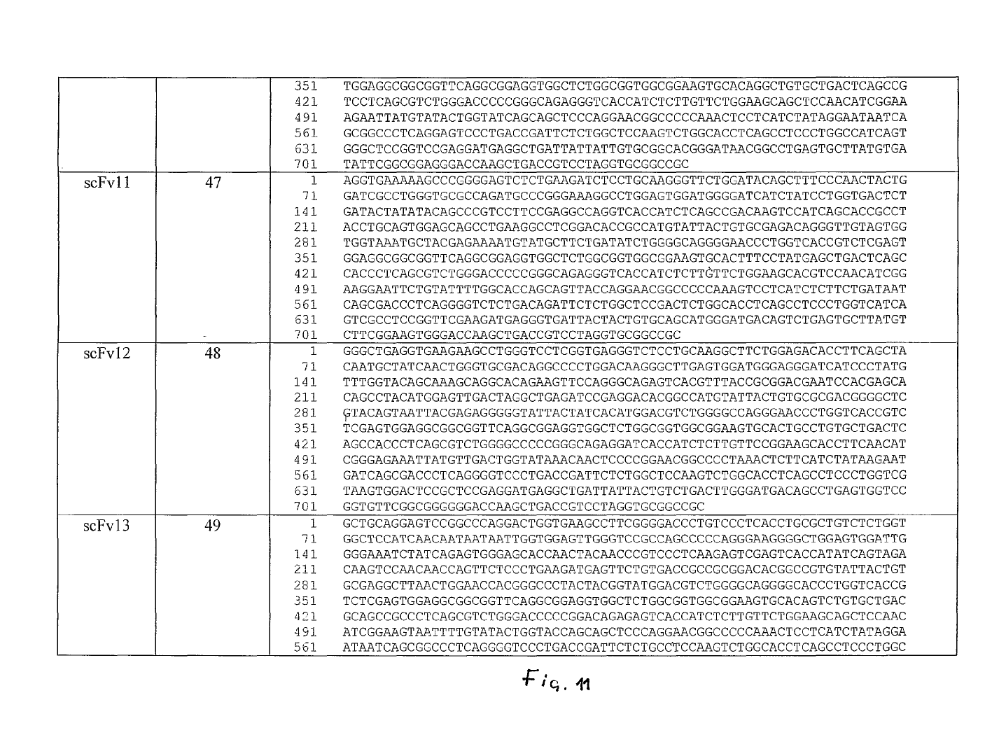

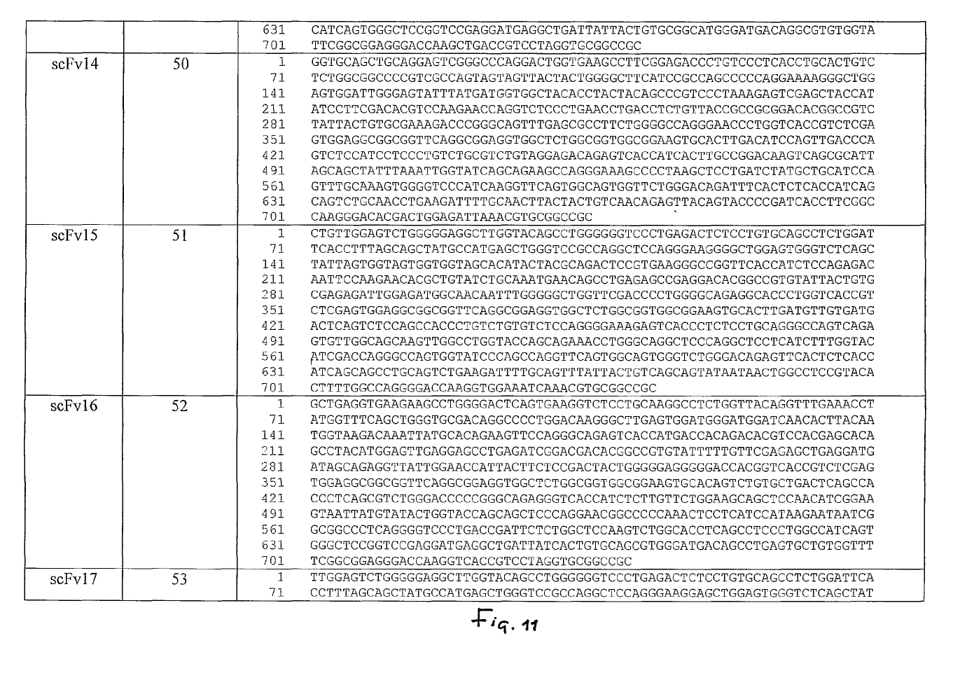

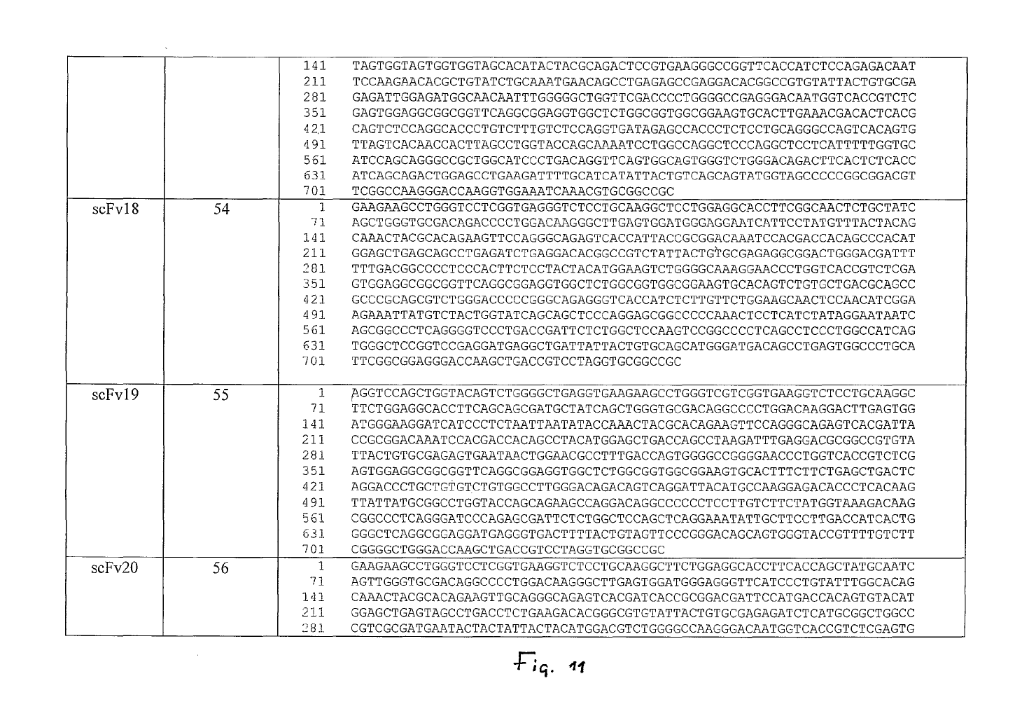

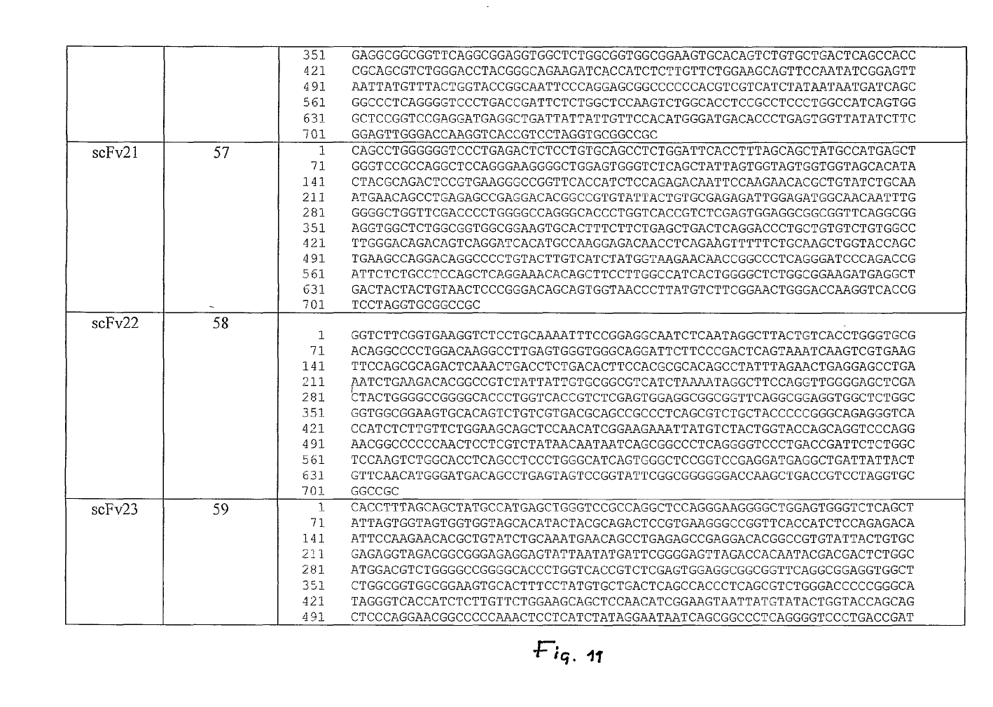

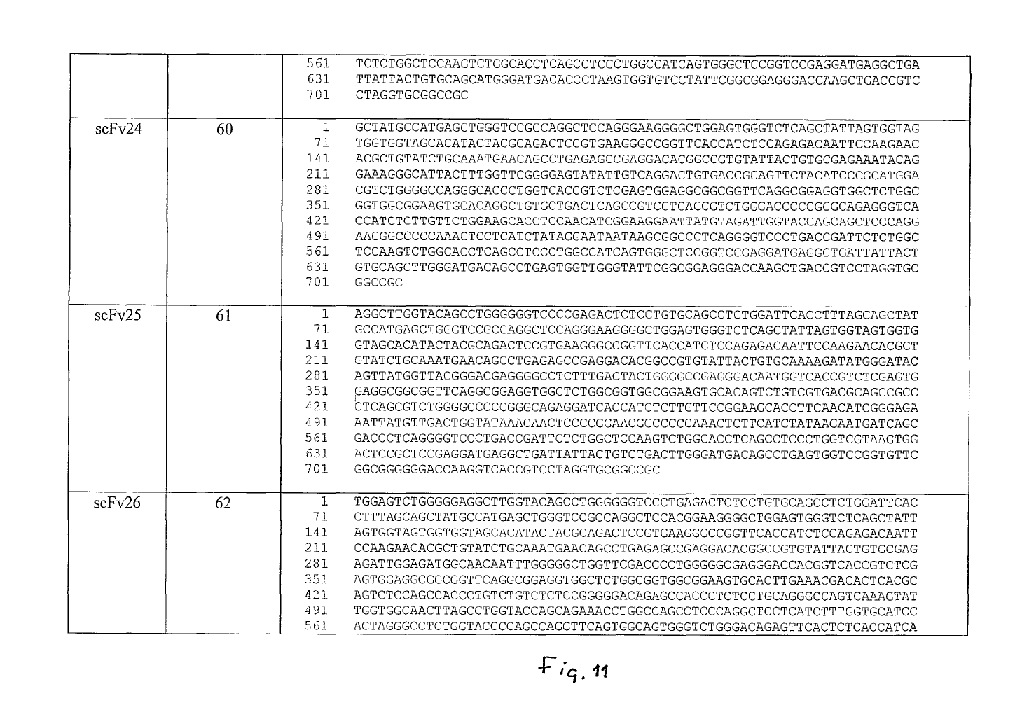

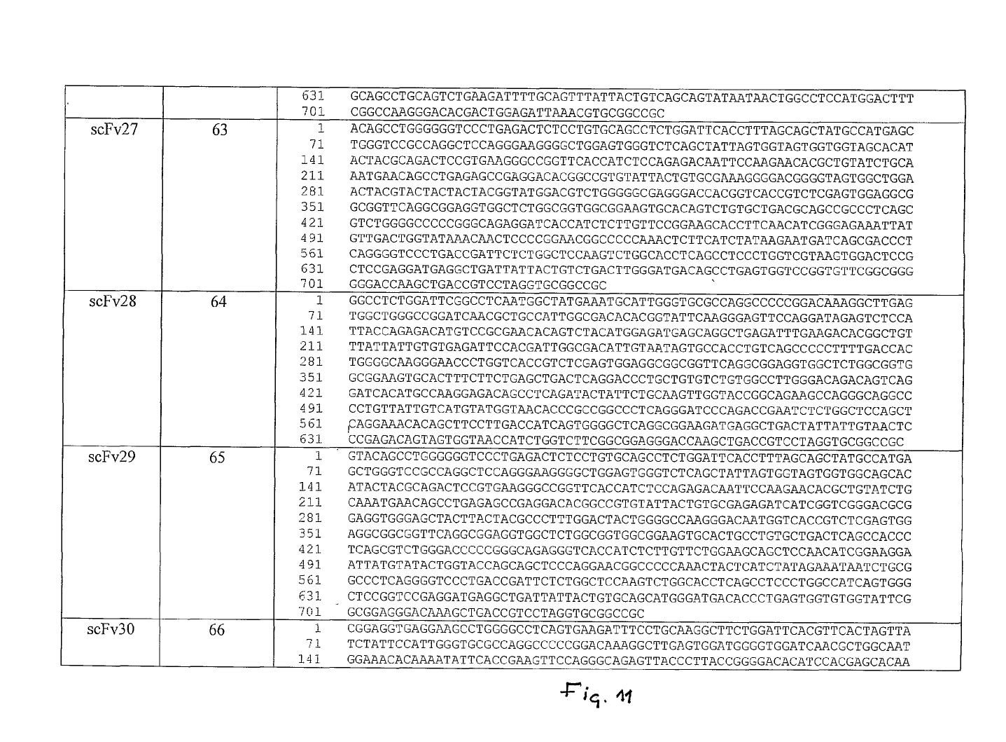

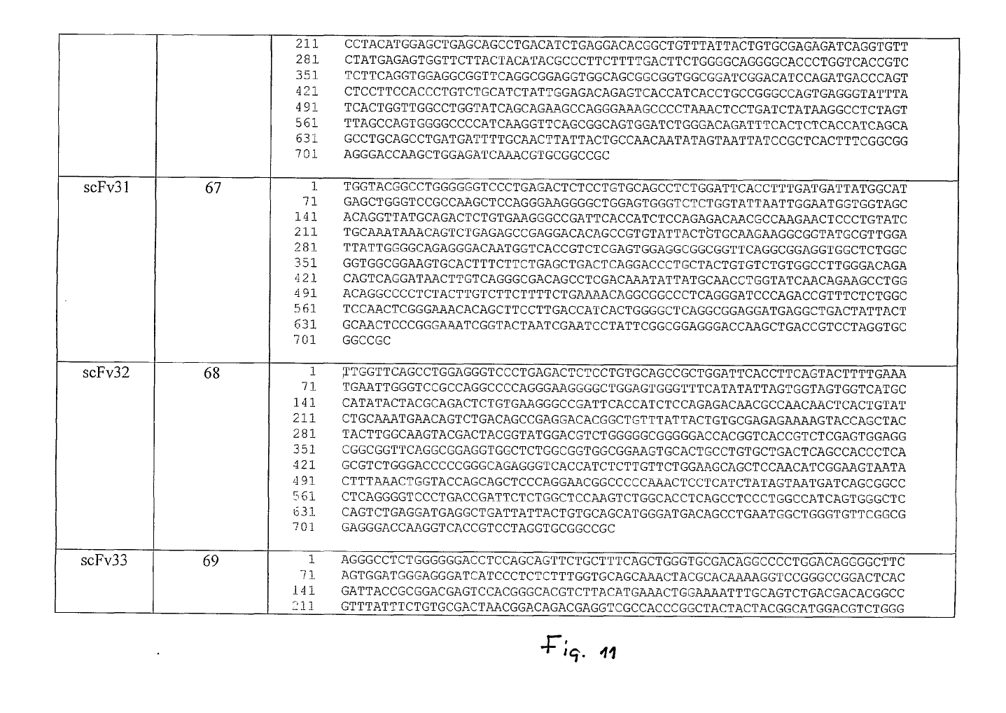

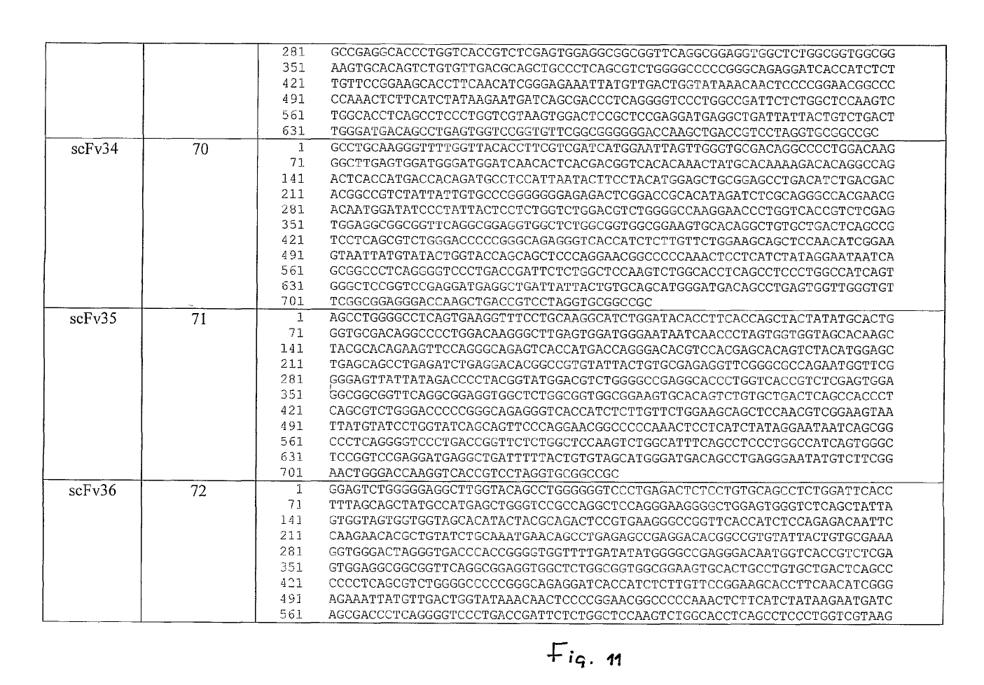

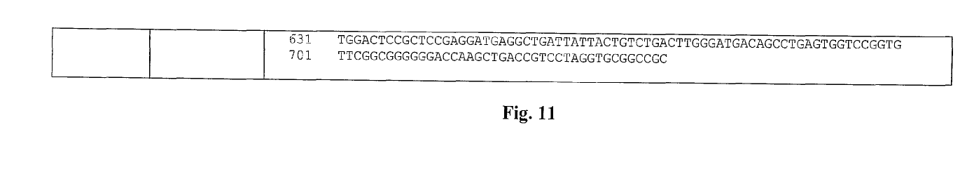

Sequencing of all scFv genes was performed by Sequiserve GmbH, Vaterstetten, Germany using the primer pXP2 Seq2 (5′-CCCCACGCGGTTCCAGC-3′) and pXP2 Seq1 (5′TACCTATTGCCTACGGC-3′). The amino acid sequences are shown in FIG. 10 and nucleotide sequences are shown in FIG. 11.

Unique clones identified by sequencing were streaked out from glycerol stocks onto LB/Amp (100μg/ml)/1% Glucose Agar plates and incubated o/n at 30° C. 10 ml LB/Amp/Glu (1%) media were inoculated with a single colony and grown o/n at 30° C. and 200 rpm shaking. The next morning the overnight cultures were is placed on ice until inoculation of 1L 2xTY media supplemented with 100 μg/ml

Ampicillin and 0.1% Glucose in 2L Erlenmeyer-flasks. The cultures were grown at 25° C. shaking until an OD₆₀₀ 0.5-0.6 was reached and then induced with IPTG 0.1 mM final concentration. Fresh Ampicillin was added to 50 μg/ml and incubation was proceeded at 22° C. o/n shaking. In the morning the cultures were centrifuged at 5000×g for 15 minutes at 4° C., supernatants discarded and the pellets resuspended carefully on ice with a pipette in 10 ml pre-cooled PBS-0.5 M Na buffer containing protease inhibitors complete (#1697498, Roche). After resuspension was completed, bacterial suspensions were transferred to 20 ml oakridge centrifuge tubes and hen-egg lysozyme (#L-6876, Sigma) added to a final concentration of 50 μg/ml for 1 hour on ice. The lysed bacteria were centrifuged at 20000×g for 15 minutes at 4° C. and the supernatants (lysate) transferred to a 15 ml plastic tube. For affinity purification the lysates were loaded with 1 ml/min onto 1 ml StrepTactin (#2-1505-010, IBA) columns equilibrated with 10 column volumes (CV) PBS-0.5 M Na buffer via a parallel protein purification system (self-made). After a 10 CV wash with PBS the elution was done with 5 CV PBS/5 mM Desthiobiotin (#D-1411, Sigma) and 1 ml fractions collected. The fractions were measured at UV₂₈₀, protein containing fractions were pooled and concentrated with Amicon Ultra Centrifugal Filter Devices 10.000 MWCO (#UFC801024, Millipore) at 4700×g. The concentrated scFv were checked on 12% Bis-Tris SDS-PAGE gels stained with Coomassie. Blue for purity and frozen in aliquots with 20% glycerol at −80° C.

### Example 4b

### Cloning of scFv into IgG Format and Expression

The scFvs consist of the sequence of a variable light and heavy chain linked by a linker sequence. The variable light chain and the variable heavy chain were amplified by PCR separately with the usage of primer, which contain restriction sites. Those restriction sites are also present in the vectors, which contain the appropriate constant domains for the heavy and light chain. The amplified variable domains were cut with the restriction enzymes and cloned into the cut vectors. The correct sequence was confirmed via sequencing

Four vectors were used, one contained the constant domain of the heavy chain for IgG1 format. The second contained the constant domain of the heavy chain for IgG4 format and two contained the constant domain of lambda and kappa light chains, respectively. Different restriction sites enabled to cut the vectors and to ligate the variable domains in the vectors.

For expression of the IgGs in mammalian cell lines the vectors contained an Epstein Barr virus origin of replication (oriP sequence) which enhances the level of transcription in 293-EBNA-HEK cells, because the EBNA protein leads to the replication of the episomal vector.

A co-transfection was carried out with the vector for the heavy chain and the vector for the light chain leading to the expression of both chains in the cell and the assembly of the IgG in the Endoplasmic Reticulum. The assembled IgG was then secreted to the medium. As transfection method Calcium-phosphate transfection was used, where a precipitate of Calcium-phosphate and the DNA is formed and incorporated into the cell. After the transfection the medium was changed to serum-free medium. Three harvests per IgG were done every 3 days. The supernatant (media) were sterile-filtrated and stored at 4° C.

For the purification of the IgGs the supernatants were purified via Protein A Sepharose either by gravity flow or by HPLC depending on the volume. For up to 200 ml a gravity flow method was used. For both purification types the supernatant was loaded on the Protein A column, washed with 50 mM Tris pH 7 buffer and eluted with 0.1 M Citrate pH ˜2.To the elution fraction 0.25 M Tris pH 9 was added leading to a pH of 5.5-6.0. Depending on the further use of the IgGs they were dialysed against PBS buffer and stored at −20° C.

### Example 5

### FACS Analysis for Tumor Cell Specific Binding

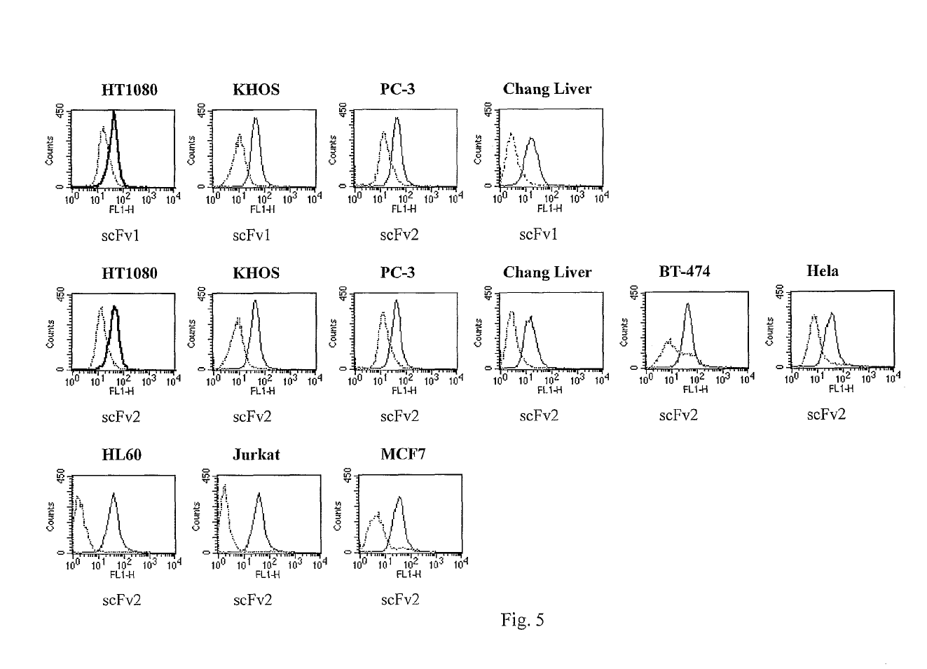

To test the ability of purified anti HT1080 single chain Fv to bind specifically to target cells, we performed a fluorescence-activated cell sorter (FACS) analysis using HT1080 cells (ATCC CCL-121), KHOS cells (ATCC CRL-1544), PC-3 cells (ATCC CRL-1435), BT-474 cells (ATCC HTB-20), Hela cells (DSMZ ACC-57), HL60 cells (DSMZ ACC-3), Jurkat cells (DSMZ ACC-282), MCF7 cells (DSMZ ACC-115) and chang liver cells (DSMZ ACC-57 contain impurities of Hela cells) (for all 10⁶ cells/ml) and Hs-27 cells (10⁶ cells/ml) as control cell line (see FIG. 5). Cells were incubated with 10 μg/ml of pure scFv in CellWash (BD (Becton, Dickinson and Company) #349524) for 20 min at 4° C., washed, and bound scFvs were detected with a secondary FITC labeled anti E-tag mab (Amersham #27-9412-01). Samples were washed and analyzed on a Becton Dickinson FACSscan. FIG. 5 shows the log fluorescence intensity (FL1-H; x-axis) versus the relative cell numbers (counts; y-axis) for cells reacting with scFv1. The thin line represents the control cell line (HS-27) and the bold line the tumor cell lines or chang liver cells. scFv1 and scFv2 specifically stain the tumor cell lines with up to 10 fold higher signals compared to the control cell line.

### Example 6

### Competition Analysis by FACS

To test the ability of the purified anti neuropilin-1 scFv to block common neuropilin-1 epitopes on the target cells, single cell suspensions of HT1080 are harvested with 0.5 mM EDTA/PBS. Approximately 1×10⁶ cells are incubated in CellWash “(BD, #349524) with 10 μg/ml scFv for one hour at 4° C. After washing with Cell Wash 10 μg/ml FITC labeled scFv is added and incubated for 20 min at 4° C. Signals of bound FITC labeled scFvs with and without pre incubation of other neuropilin-1 binders are analyzed on a Becton Dickinson FACSscan.”

### Example 7

### Labeling of Antibody Fragments with FITC

scFv were labeled with fluorescein isothiocyante (FITC) (Molecular Probes, Eugene, USA #F1906) by the following method: Aliquots of a 10 mg/ml solution of FITC in dimethyl sulfoxide were added to 100 μg of scFv1 dissolved in PBS/0.5M NaHCO₃, pH 9.5 in a ratio of 30:1 (FITC:scFv1). The sample was incubated for two hours at room temperature with agitation, free FITC was separated using desalting columns (2 Micro Spin G-25, Pharmacia 27-5325-01). The ratio of labeling was determined via mass spectrometry and via UV/VIS spectroscopy, whereby the protein concentration was calculated at 280 nm and the FITC concentration at 494 nm.

### Example 8

### Invasion Assay for Identification of Inhibitory Antibody Fragments

The ChemoTx® system (Neuro Probe Inc.#106-8, Gaithersburg) is used as a disposable chemotaxis/cell migration chamber in a 96 well format with an 8 μm filter Track etched Polycarbonate pore size, 5.7 mm diameter/site.

13.3 μl of 0.3 mg/ml Matrigel (Matrigel is a solubilized basement membrane preparation extracted from the Engelbreth-Holm-Swarm (EHS) mouse sarcoma, a tumor rich in extracellular matrix proteins. Its major component is laminin, followed by collagen IV, heparan sulfate proteoglycan, entactin and nidogen. It also contains TGF-_{—} fibroblast growth factor, tissue plasminogen activator, and other growth factors which occur naturally in the EHS tumor) (Becton Dickenson, BD #356234) diluted in Dulbeccos PBS (Gibco #14040-091) is applied on the membrane filter of the 96-well plate on row B-H and on row A 1.2 μg/site of collagen S type I (Roche #10982929) is diluted in 0.05 M HCl (Sigma #945-50) and is incubated over night at 20° C. in a desiccator for gelation. HT1080 cells are grown to 70-80% confluence in DMEM supplemented with GlutamaxI (862 mg/l (Gibco #31966-021) with 10% FCS (Gibco #10270106). The cells are washed 2× with DMEM/GlutamaxI/0.1% BSA (Sigma #A-7030) then labeled in situ with Bis-benzimide H 33342 (Sigma #B-2261) are diluted 1:100 in DMEM/GlutamaxI/0.1% BSA for 15 min at 37° C., 7.5% CO₂. Cells are washed 2× with DMEM/GlutamaxI/0.1% BSA and are loaded with DMEM/GlutamaxI/0.1% BSA for 15 min at 37° C., 7.5% CO₂ for recovering. After washing 2× with PBS w/o Ca²⁺, Mg²⁺ (Gibco, 10010-015), the cells are detached with 0.5 mM EDTA (Sigma #E8008), are collected with Dulbeccos PBS/0.1% BSA/10 mM Hepes (Gibco #15630-056), are washed 2× with Dulbeccos PBS/0.1% BSA/10 mM Hepes, are suspended in Dulbeccos PBS/0.1% BSA/10 mM Hepes and are diluted to 6.7×10⁶ cells/ml with Dulbeccos PBS/0.1% BSA/10 mM Hepes. 6.7×10⁶ cells/ml are incubated 1:1 with 40 μg/ml of a control scFv as a negative control for inhibition of invasion and with HT1080 specific scFv for 1 h on ice. After dilution to 6.7×10⁵ cells/ml with DMEM/GlutamaxI/0.1% BSA, HT1080 cells and HT1080 cell/scFv dilutions are pipetted in triplicate onto the chemotaxis chamber (row B-H) at a density of 3.4×10⁴ cells/well and are incubated for 6 h at 37° C., 7.5% CO₂. DMEM/GlutamaxI with 5% FCS is used as a chemo attractant in the lower chamber. A standard curve from 1×10⁴ to 4×10⁴ cells/site is performed on collagen S type I coated row A of the chemotaxis chamber. DMEM/GlutamaxI/0.1% BSA is used in the lower chamber (cells are not migrating). After scraping the non-migrating cells from the top of the membrane (except the Standard curve on row A) fluorescence of cells, which had migrated through the membrane (not migrated in case of the Standard curve), is measured on the Fluostar Galaxy (bMG) microplate reader using excitation/emission wavelengths of 370/460 nm.

### Example 8.1

### Influence of VEGF on the Invasion of HT1080 Cells

The invasion assay set-up was identical to the set-up described in Example 8. The invasion membrane (Neuroprobe #106-8, Gaithersburg, Md.) was coated overnight with Matrigel (0.3 mg/ml; BD #356234, Bedford, Mass.). 80% confluent HT1080 cells were stained with Bis-benzimide H33342 and then detached with 0.05% EDTA. Cells (7×10⁶/ml, final 35.000/well) were pre-incubated for 1 h at 4° C. with:
a. 0, 25, 50 or 100 ng/ml recombinant human VEGF (R&D Systems 4293-YE, Minneapolis) in the presence or absence of 20 μg/ml anti-human VEGF antibody (R&D Systems #AF-293-NA). 5% FCS (not heat-inactivated) in DMEM was used as chemo-attractant in the well below the membrane;
b. in the presence or absence of 20 μg/ml anti-human VEGF antibody, with 5% FCS and 25, 50 or 100 ng/ml VEGF as chemo-attractant;

c. 0, 25, 50 or 100 ng/ml VEGF in the presence or absence of 20 μg/ml anti-human VEGF antibody, with 1% BSA (Sigma #A7030, ST. Louis, Mo.) in DMEM in the well below the membrane;
d. in the presence or absence of 20 μg/ml anti-human VEGF antibody, using 25, 50 or 100 ng/ml VEGF in DMEM containing 1% BSA as chemo-attractant.

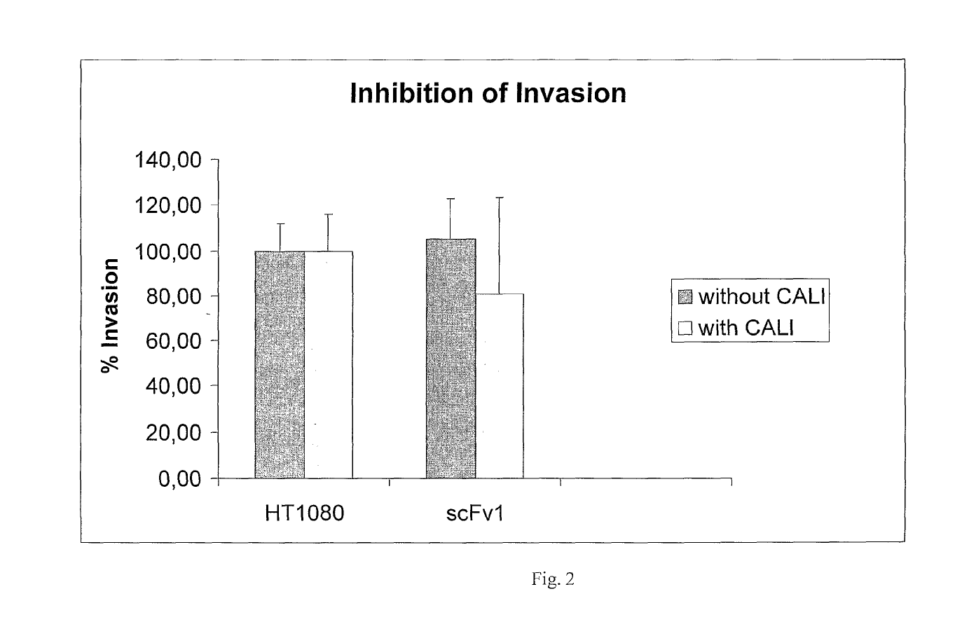

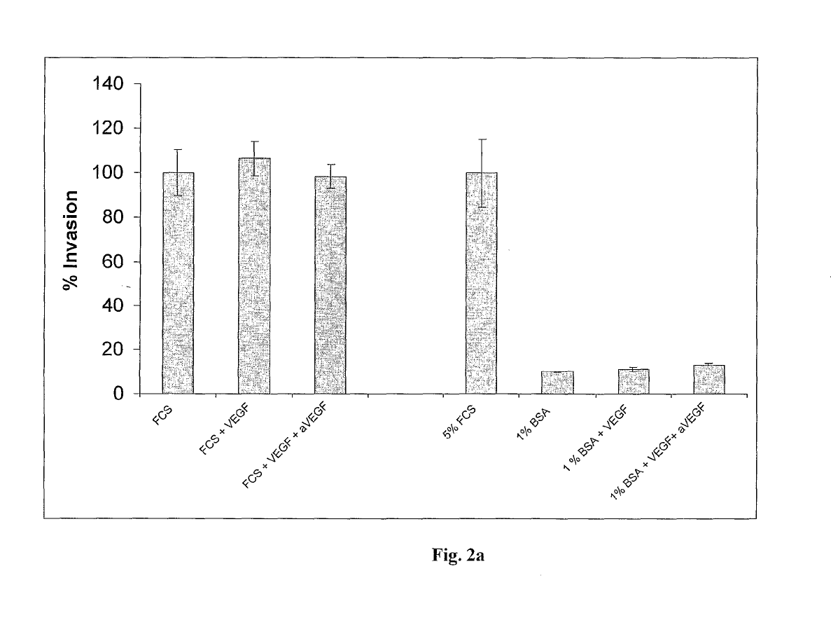

50 μl of the preincubated cell suspensions (a-d) were pipetted on top of the membrane and the invasion plate was incubated for 6 h at 37° C., 7.5% CO₂. The readout of the invasion assay was identical to the readout described in Example 8. No significant increase of invasion was seen when the cells were pre-incubated with different concentrations of recombinant VEGF (a). Also, the addition of anti-human VEGF antibody had no influence on the invasion of HT1080 cells (a), compared to the result of Experiment 8. The addition of recombinant VEGF to the chemo-attractant FCS did not result in an increase of invasion of HT1080 cells (b). A variation of the VEGF concentration did not show an effect either. The addition of anti-human VEGF antibody had also no effect on this result (b). The pre-incubation with VEGF did not result in an invasion of HT1080 cells when BSA was used as a chemo-attractant (c). The use of VEGF as a chemo-attractant in DMEM and 1% BSA did also not stimulate the invasion of HT1080 cells (d), whereas 5% FCS as a chemo-attractant induced invasion (fourth bar from the left in FIG. 2a).

### Example 9

### Invasion Assay for Target Identification with CALI

This Example is in genial identical to Example 8, except for the use of FITC-labeled scFv (see, Example 7 for labeling) and the integration of the CALI process within the invasion assay.

The ChemoTx® system (Neuro Probe Inc.#106-8, Gaithersburg) was used as a disposable chemotaxis/cell migration chamber in a 96 well format with an 8 μm filter Track etched Polycarbonate pore size, 5.7 mm diameter/site.

13.3 μl of 0.3 mg/ml Matrigel (see Example 8) diluted in Dulbeccos PBS (Gibco #14040-091) was applied on the membrane filter of the 96-well plate on row B-H and on row A 1.2 μg/site of collagen S type I (Roche #10982929) was diluted in 0.05 M HCl (Sigma #945-50) and was incubated over night at 20° C. in a desiccator for gelation. HT1080 cells were grown to 70-80% confluence in DMEM supplemented with GlutamaxI (862 mg/l (Gibco #31966-021) with 10% FCS (Gibco #10270106). The cells were washed 2× with-DMEM/GlutamaxI/0.1% BSA (Sigma #A-7030) then labeled in situ with Bisbenzimide H 33342 (Sigma #B-2261) and were diluted 1:100 in DMEM/GlutamaxI/0.1% BSA for 15 min at 37° C., 7.5% CO₂. Cells were washed 2× with DMEM/GlutamaxI/0.1% BSA and loaded with DMEM/GlutamaxI/0.1% BSA for 15 min at 37° C., 7.5% CO₂ for recovering. After washing 2× with PBS w/o Ca²⁺, Mg²⁺ (Gibco, 10010-015), the cells were detached with 0.5 mM EDTA (Sigma #E8008), collected with Dulbeccos PBS/0.1% BSA/10 mM Hepes (Gibco #15630-056), washed 2× with Dulbeccos PBS/0.1% BSA/10 mM Hepes, suspended in Dulbeccos PBS/0.1% BSA/10 mM Hepes and diluted to 6.7×10⁶ cells/ml with Dulbeccos PBS/0.1% BSA/10 mM Hepes. 6.7×10⁶ cells/ml were incubated 1:1 with 40 μg/ml of FITC-labeled anti-betal integrin monoclonal antibody (JB1, Chemicon #MAB1963) as a control for inhibition of invasion after CALI and with HT1080 specific FITC labeled scFv for 1 h on ice. 1.3×10⁵ HT1080 cells/well or HT1080 cell/scFv or Ab dilution were pipetted in triplicate in two 96-well plate, black, ultra thin clear flat bottom special optics (Costar #3615). One plate was kept on ice in the dark while the other plate was irradiated on an ice block with continuous wave laser at 488 nm (0.5 W, 30 sec). After dilution to 6.7×10⁵ cells/ml with DMEM/GlutamaxI/0.1% BSA, HT1080 cells and HT1080 cell/scFv dilutions were pipetted in triplicate (non irradiated triplicate beside irradiated triplicate) onto the chemotaxis chamber (row B-H) at a density of 3.4×10⁴ cells/well and incubated for 6 h at 37° C., 7.5% CO₂. DMEM/GlutamaxI with 5% FCS was used as a chemo attractant in the lower chamber. A standard curve from 1×10⁴ to 4×10⁴ cells/site was performed on collagen S type I coated row A of the chemotaxis chamber. DMEM/GlutamaxI/0.1% BSA was used in the lower chamber (cells were not migrating). After scraping the non-migrating cells from the top of the membrane (except the Standard curve on row A) fluorescence of cells, which had migrated through the membrane (not migrated in case of the Standard curve), was measured on the Fluostar Galaxy (bMG) microplate reader using excitation/emission wavelengths of 370/460 nm. In a general experiment, a value of 45000 corresponded to 100% migrated cells.

The invasion phenotype of HT1080 cells was assessed by comparing their relative ability to invade tumor extracellular matrix (Matrigel) using the Transwell culture system described above. scFv1 showed after CALI an inhibitory effect of 25% on the invasion of the HT1080 cells. The result of the invasion assay with and without CALI (see Example 8) is shown in FIG. 2. FIG. 2 shows that CALI converts scFv1 in an inhibitory antibody fragment.

### Example 10

### MTS Viability Assay

Viable cells were detected by measuring the conversion of the tetrazolium dye MTS (MTS, Celltiter A_{queous} one, Promega #G4000) to formazan. HT1080 cells and HT1080 cell/scFv dilutions (obtained from the dilutions prepared in the Invasion assay) were pipetted in triplicate at a density of 3.4×10⁴ cells/well and were plated in a 96-well plate (black, ultra thin clear flat bottom, special optics, Costar #3615) 10 μl MTS was added to each well and incubated for 1 hour at 37° C., 7.5% CO₂. Absorbance was measured at 492 nm with the Fluostar Galaxy (bMG) microplate reader. For all tested scFvs, no effect on viability of cells was seen (data not shown).

### Example 11

### Cell-Matrix Adhesion Assay for Identification of Inhibitory Antibody Fragments

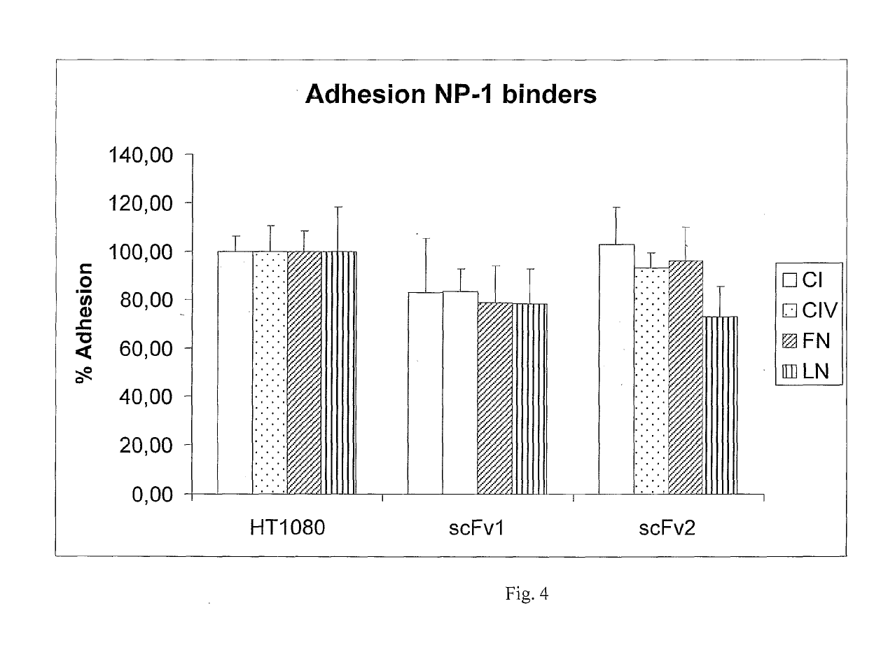

21 wells of a 96-well flat bottom plate (Costar #3614) were coated with one matrix protein selected from collagen S type I 1 μg/well (Roche #10982929), collagen type IV 1 μg/well (Rockland 009-001-106), fibronectin 1 μg/well (Sigma F2518) and laminin 1 μg/well (Roche 1243217) in Dulbeccos PBS (Gibco #14040-091), respectively, at 4° C. over night. At the same time 3 wells in row A were coated with 2% BSA (Sigma #A-7030)/Dulbeccos PBS for a blank value. Wells were washed twice with Dulbeccos PBS, and blocked with 2% BSA (Sigma #A-7030)/Dulbeccos PBS for 1 h at 37° C. and washed with Dulbeccos PBS. HT1080 were harvested, stained with 2.5 mM (final concentration) Calcein AM (Molecular Probes C-3099), washed twice with PBS w/o CaCl₂ w/o MgCl₂ (Gibco 10010-015) and diluted to 1.5×10⁵/ml in buffer (0.5% BSA (Sigma #A-7030)+10 mM Hepes DMEM (Gibco 31966-02)). HT1080 cells were mixed with 10 μg/ml scFv and incubated for 30 min on ice. The HT1080 cells alone and HT1080/scFv dilutions were pipetted in triplicate at a density of 1.5×10⁴ cells/well and incubated for one hour at 37° C., 7.5% CO₂. After two additional washing steps with Dulbeccos PBS, where non-adherent cells were washed away, a Standard curve from 3.7×10³ to 1.5×10⁴ stained cells/well diluted in Dulbeccos PBS was performed in triplicate in row A. Washed wells were filled with 100 μl Dulbeccos PBS and the absorbance of attached cells and of the Standard curve was measured on the Fluostar Galaxy (bMG) microplate reader using excitation/emission wavelengths of 485/520 nm. scFv1 showed an inhibitory effect of approximately 20% on the adhesion of HT1080 cells to collagen S type I, collagen type IV, fibronectin and laminin. scFv2 showed an inhibitory effect of 27% on the adhesion of HT1080 cells to laminin and a relatively low inhibitory effect on the adhesion of HT1080 cells to collagen type IV and fibronectin. No effect was seen on the adhesion of HT1080 cells to collagen S type I. Results for scFv1 and scFv2 are shown in FIG. 4.

### Example 12

### Cell-Matrix Adhesion Assay for Target Identification with CALI

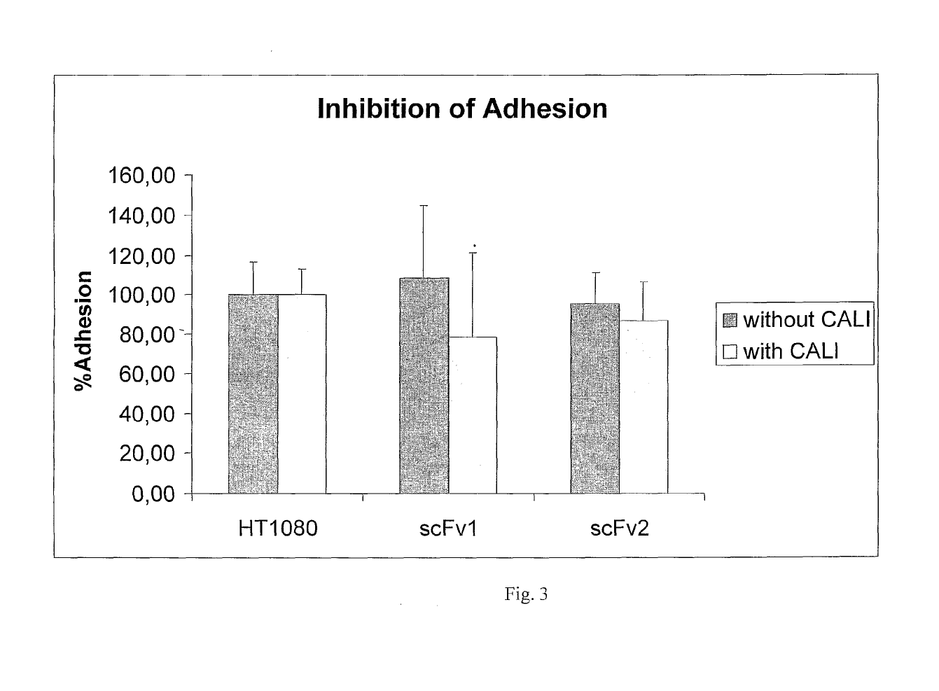

96-well plates (TPP #9296) (cell culture treated) were coated in Row B-H with collagen S type 1 μg/well (Roche #10982929) in Dulbeccos PBS (Gibco #14040-091) and in Row A well 10-12 were coated with 2% BSA (Sigma #A-7030)/Dulbeccos PBS at 4° C. over night. The plate was washed with Dulbeccos PBS, blocked Row B-H and Row A well 10-12 with 2% BSA/Dulbeccos PBS for 1 h at 37° C. and washed again with Dulbeccos PBS. HT1080 cells were grown to 70-80% confluence in DMEM supplemented with GlutamaxI (862 mg/l (Gibco #31966-021) with 10% FCS (Gibco #10270106). The cells were washed 2× with DMEM/GlutamaxI/0.1% BSA (Sigma #A-7030) then labeled in situ with Bis-benzimide H 33342 (Sigma #B-2261) were diluted 1:100 in DMEM/GlutamaxI/0.1% BSA for 15 min at 37° C., 7.5% CO₂. Cells were washed 2× with DMEM/GlutamaxI/0.1% BSA and loaded with DMEM/GlutamaxI/0.1% BSA for 15 min at 37° C., 7.5% CO₂ for recovering. After washing 2× with PBS w/o Ca²⁺, Mg²⁺ (Gibco, 10010-015), the cells were detached with 0.5 mM EDTA (Sigma #E8008), collected with Dulbeccos PBS/0.1% BSA/10 mM Hepes (Gibco #15630-056), washed 2× with Dulbeccos PBS/0.1% BSA/10 mM Hepes, suspended in Dulbeccos PBS/0.1% BSA/10 mM Hepes and diluted to 6.7×10⁶ cells/ml with Dulbeccos PBS/0.1% BSA/10 mM Hepes. 6.7×10⁶ cells/ml were incubated 1:1 with 40 μg/ml of FITC-labeled anti-betal integrin monoclonal antibody (JB1, Chemicon #MAB1963) as a control for inhibition of adhesion after CALI and with HT1080 specific FITC labeled scFv for 1 h on ice. 1.3×10⁵ HT1080 cells/well or HT1080 cell/scfv or Ab dilution were pipetted in triplicate in two 96-well plate, black, ultra thin clear flat bottom special optics (Costar #3615). One plate was kept on ice in the dark while the other plate was irradiated on an ice block with continuous wave laser at 488 nm (0.5W, 30 sec). After dilution to 6.7×10⁵ cells/ml with DMEM/GlutamaxI/0.1% BSA, HT1080 cells and HT1080 cell/scFv dilutions were pipetted in triplicate (non irraditated triplicate beside irradiated triplicate) onto the coated and blocked plate. In Row A well 10-12 6.7×10⁵ cells/ml with DMEM/GlutamaxI/0.1% BSA were pipetted as a to background control. Plate was incubated for 1 h at 37° C., 7.5% CO₂ and washed 2× with Dulbeccos PBS, where non-adherent cells were washed away. In Row A well 1-9 a standard curve from 1×10⁴to 4×10⁴ cells/well is performed, in all other wells 50 μl Dulbeccos PBS is pipetted. Fluorescence of cells, which had adhered to the Collagen S type I (not adhered in case of the Standard curve), was measured on the Fluostar Galaxy (bMG) microplate reader using excitation/emission wavelengths of 370/460 nm. scFv1 showed after CALI an inhibitory effect of 30% on the adhesion of the HT1080 cells to collagen S type I. scFv2 showed after CALI an inhibitory effect of approximately 10% on the adhesion of HT1080 cells to collagen S type I. In one set of experiments scFv2 showed already an inhibitory effect on the adhesion of HT1080 cells to collagen S type I of 5% without CALL The results of the adhesion assay with and without CALI are shown in FIG. 3.

### Example 13

### Immunoprecipitation

HT1080 and Hs-27 cells (10⁸) were lysed in 3 ml 50 mM Tris-HCl, pH 8.0, 150 mM NaCl, 1% Triton X-100 (v/v) containing protease inhibitor cocktail (1 pill in 50 ml buffer) (Boehringer Mannheim, Cat.-No. 1697498) and 100 μM Pefablock (Roth, Cat.-No. A154.1). Lysates were pre-incubated for 2 h at 4° C. with Streptactin Sepharose (IBA, #2-1201-010) and the supernatants used for the immunoprecipitation reactions. HT1080 specific scFv (50 μg/l mg cell extract) were added to the cleared lysates, samples rotated for 2 h at 4° C., gently centrifuged at 700 g to pellet the Streptactin Sepharose, the pellet was washed 4 times with 1 ml volume of PBS+0.1% Tween buffer per wash, before the complexes were isolated by elution from the Streptactin Sepharose pellet with 50 μl 10 mM D-desthiobiotin in PBS 0.1% Tween 20. The immuno-complexes were separated by SDS-PAGE and silver stained for MS analysis.

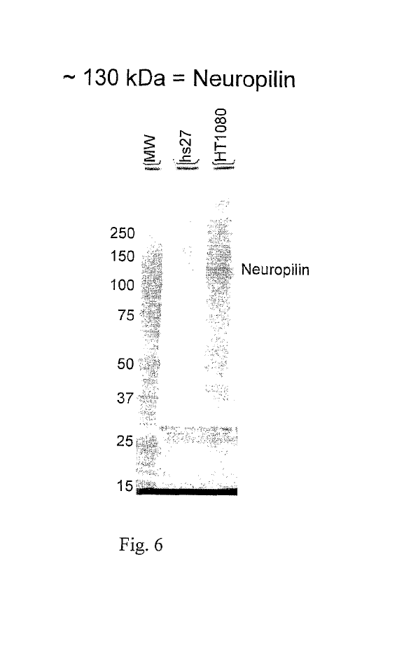

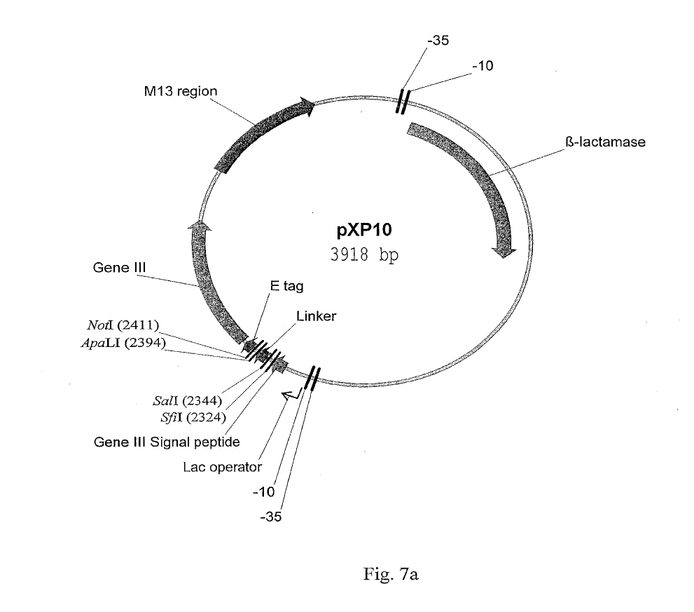

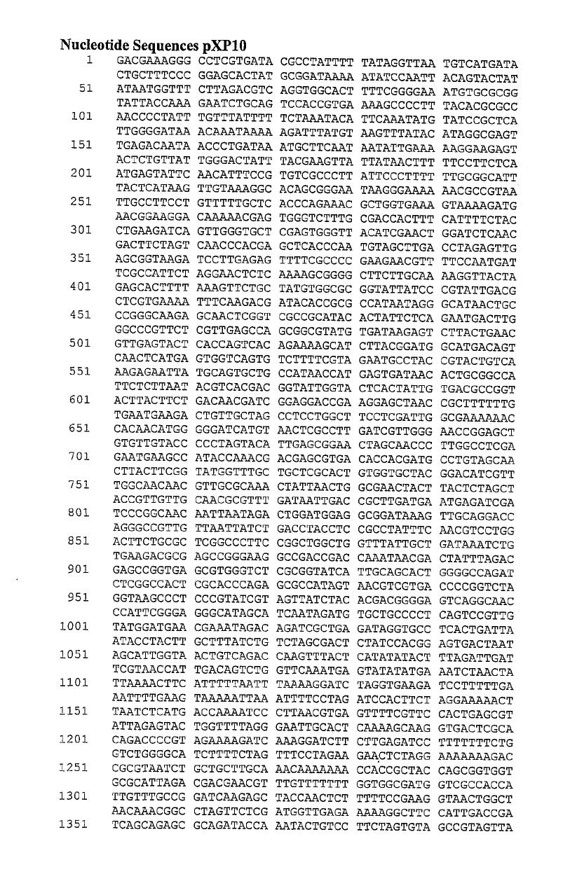

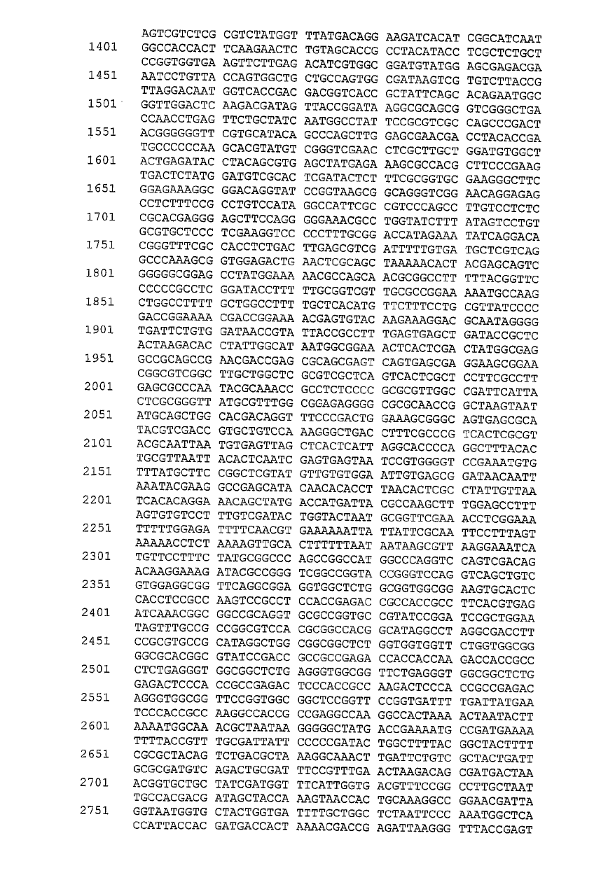

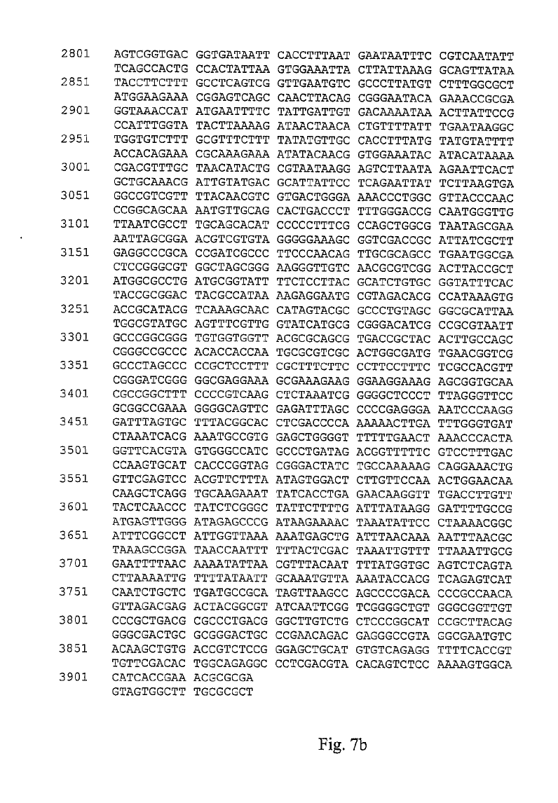

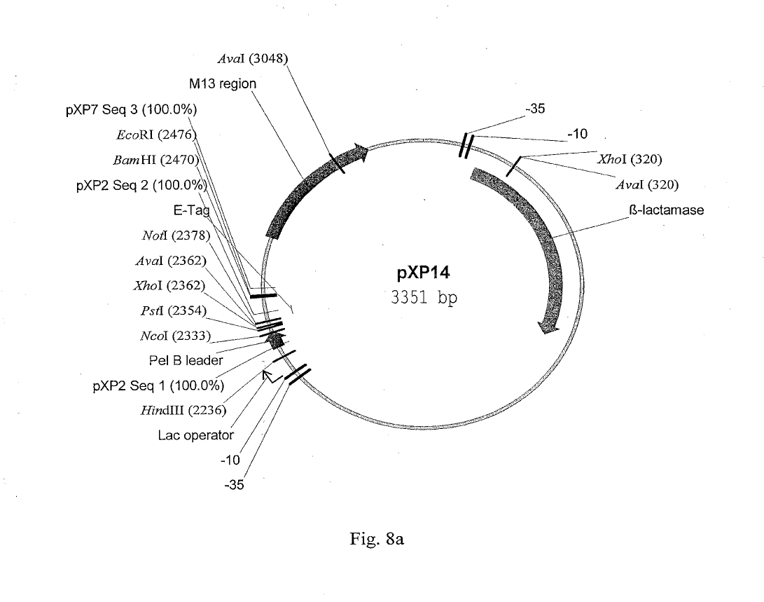

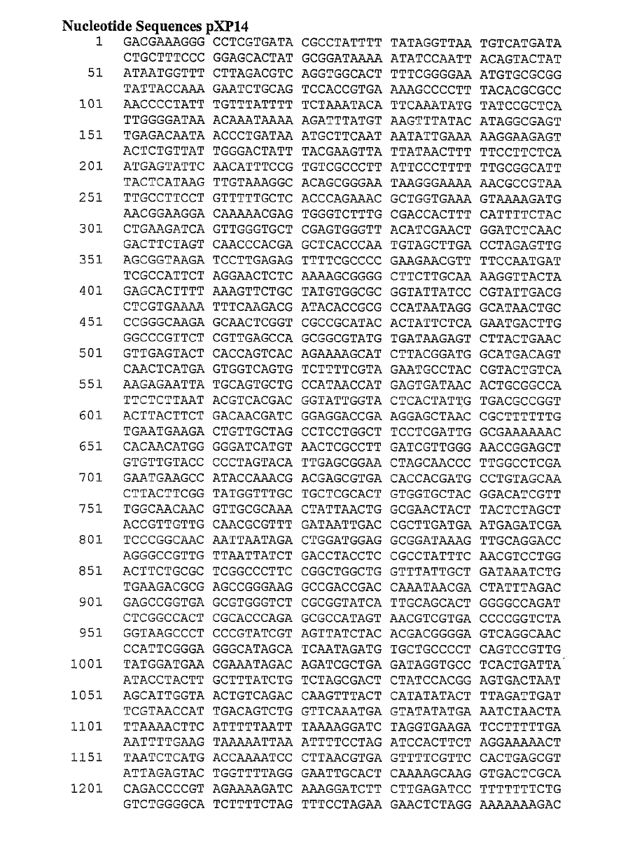

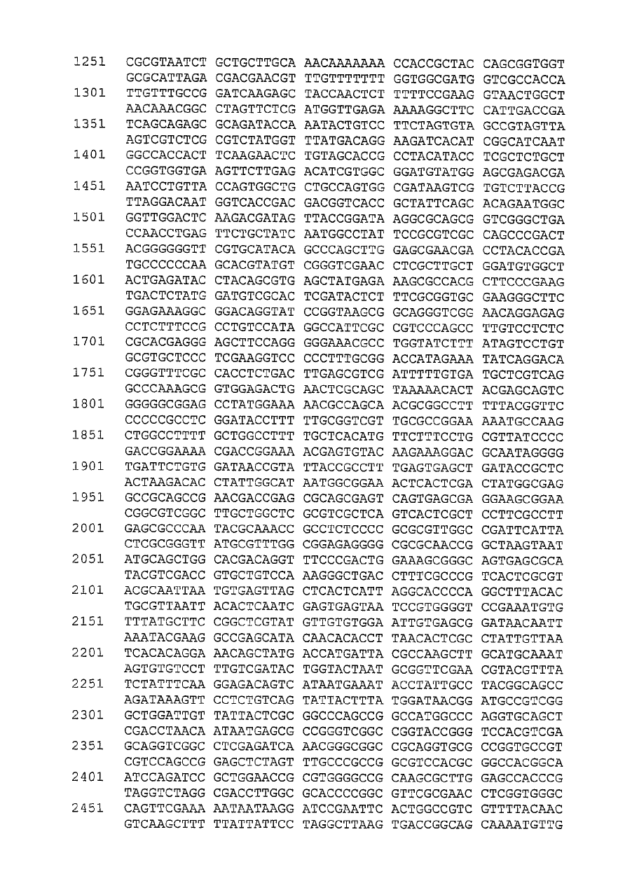

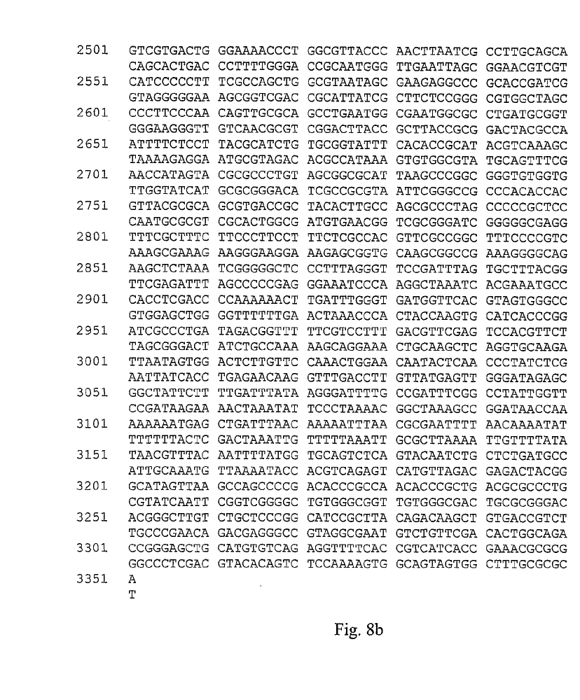

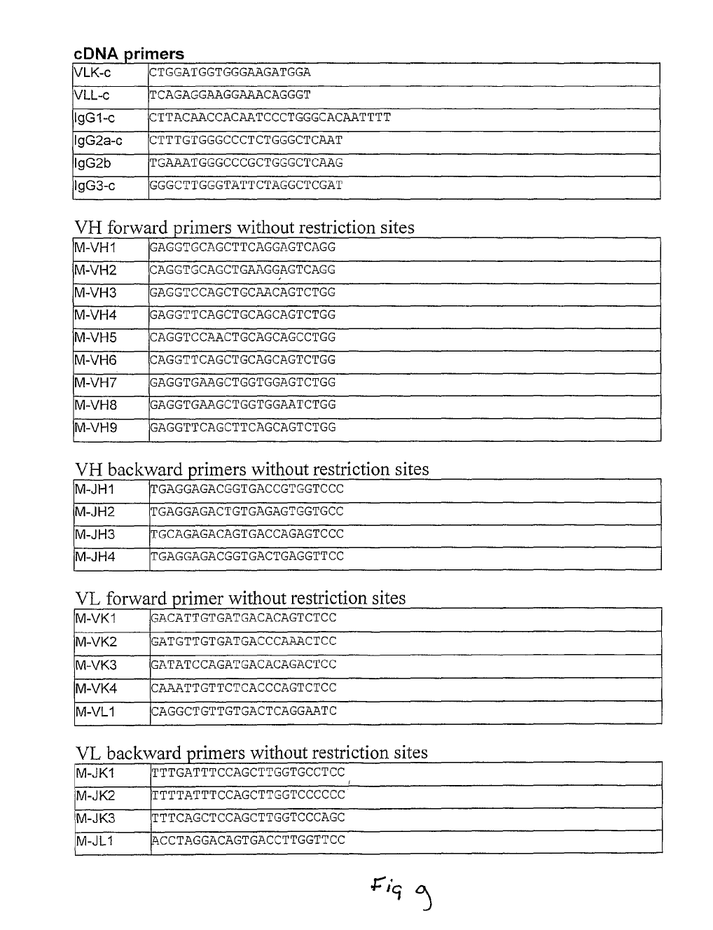

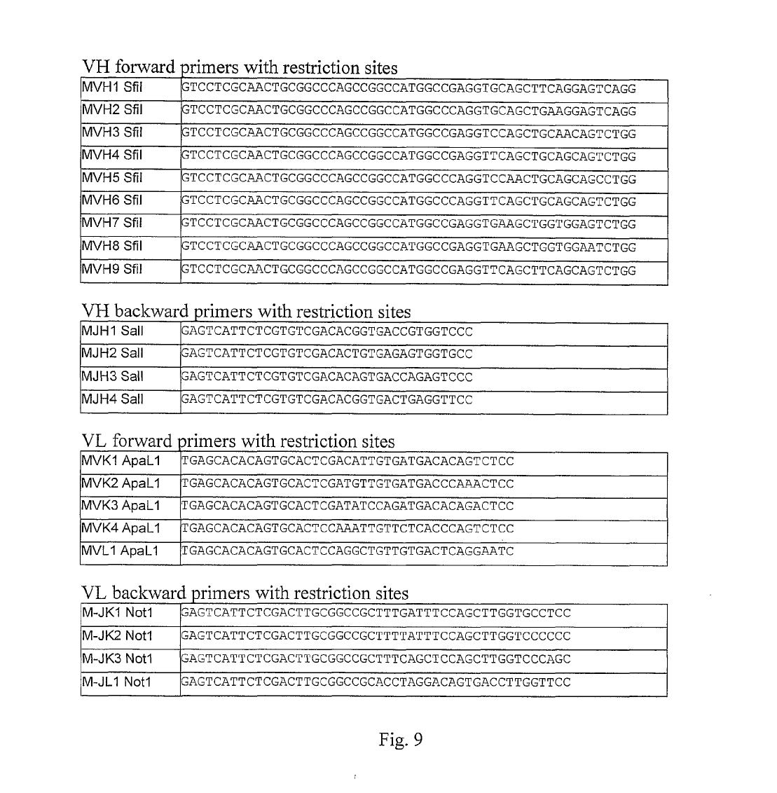

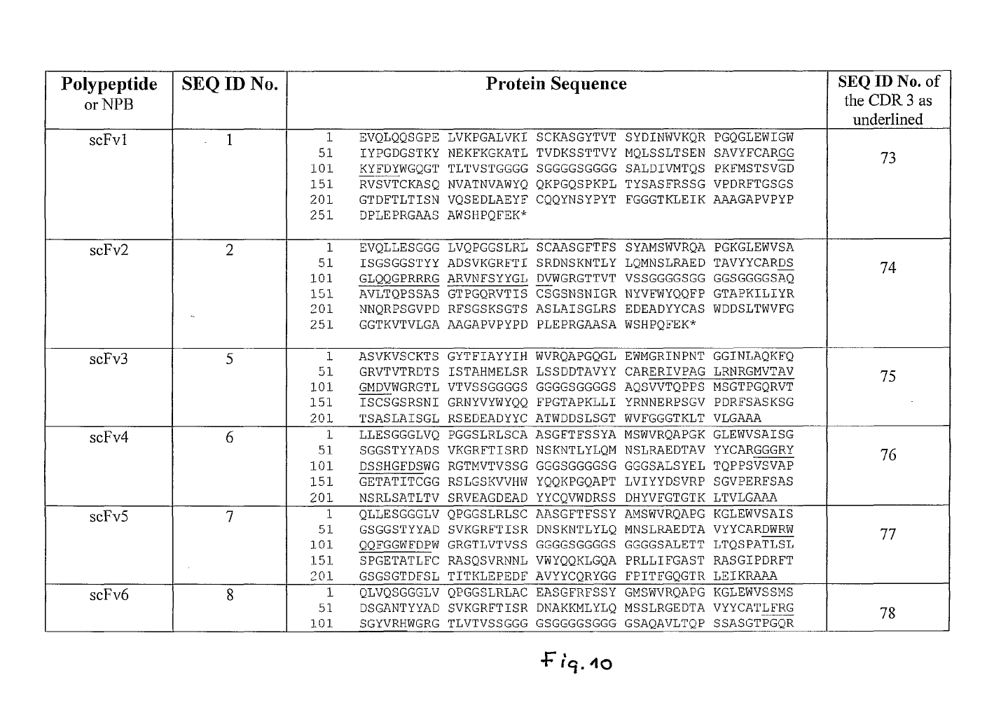

scFv 1 and scFv 2 pulled down a protein, detected as a band on SDS-PAGE by silver staining at a molecular weight of ˜130 kDa. This band was only detected in the HT1080 cell extract and not in the Hs-27 cells (control cells), see FIG. 6.

### Example 14

### Protein Identification Via Mass Spectroscopy

The gel bands obtained from immunoprecipitations followed by SDS PAGE were subjected to a tryptic in-gel digest over night at 37° C. Peptides were extracted using 5% formic acid and the resulting peptide mixture was desalted using ZipTip μC18 (Millipore) and eluted first with 2 μl of 30% ACN/0.1% TFA, then with 2 of 70% ACN/0.1% TFA. The two fractions were pooled and one microliter of the obtained peptide mixtures was mixed in a 1:1 ratio with a solution of α-cyano-4-hydroxycinnamic acid (3 mg/ml), co-crystallized on a Teflon-coated stainless steel target and analyzed on a MALDI-TOF instrument yielding peptide mass fingerprints (PMF) in a mass range of m/z 800-3000. The obtained PMF were used to search all entries for the species Homo sapiens in the NCBI and SwissProt databases. In all cases, only peptides matching a given protein with a mass deviation of less than 10 ppm were considered for identification.

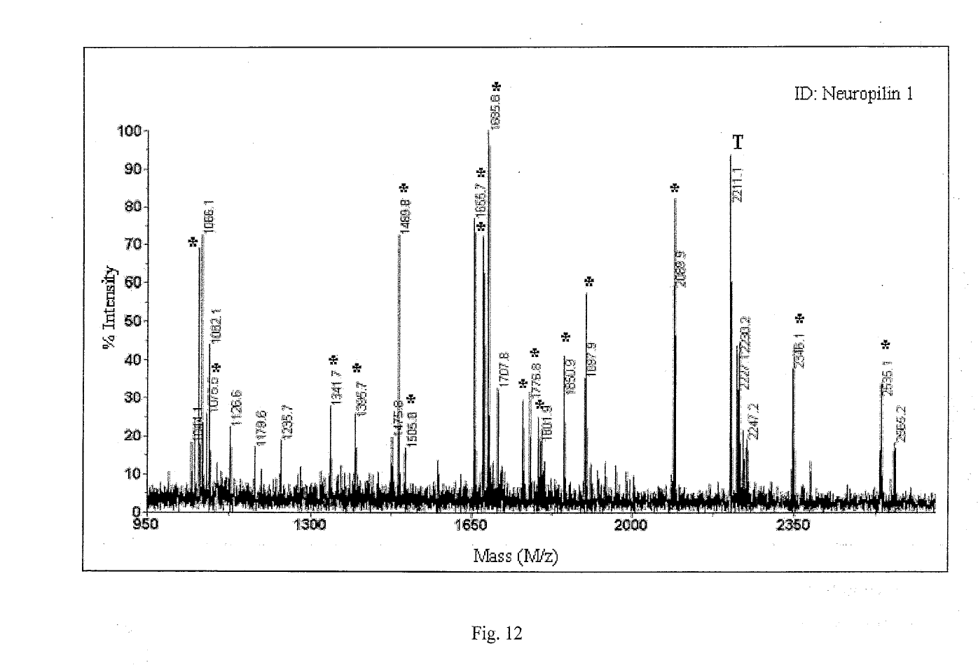

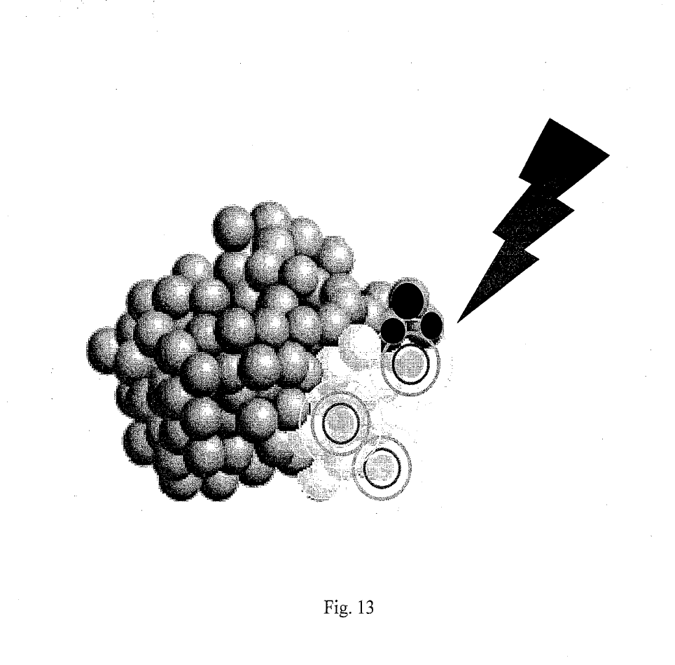

The band with an approximate size of 130 kDa, obtained by using scFv1 or scFv2, yielded up to 17 peptide peaks (in different experiments with the same scFv), which matched neuropilin-1, with a maximum protein coverage of 22% (206/923 residues). 3 peptides, namely peptide fragments 659-672, 680-702 and 776-787 clearly support the identity of neuropilin-1. Neuropilin-1's splice variant, called soluble neuropilin-1, misses amino acids 645-923 when compared to neuropilin-1 with its 923 amino acids. Therefore, the obtained mass spectrum clearly proves that the full length version of NP-1 was immunoprecipitated by scFv1 and scFv2. A spectrum is shown in FIG. 12.

### Example 15

### Methods for Epitope Mapping

An epitope mapping may be carried out according to one of the following methods:

### Example 15.1

### “Classical” Epitope Mapping

Defined fragments of the cDNA for the antigen of interest are expressed as recombinant (fusion)proteins and probed in various assays such as Westernblot or ELISA.

### Example 15.2

### Phage Display Technology

The technique of epitope mapping using random peptide phage display libraries was developed to clone small random fragments of the cDNA for the antigen of interest into the phage protein pIII of filamentous phages and display them on the surface of the phage (Fack et al., (1997) J. Immunol. Methods 7, 43-52). Epitope-displaying phages can be captured with antibodies in a procedure called “bio-panning”. Sequencing of the inserts of the corresponding phages gives some information on the epitopes. This procedure is in principle capable to identify conformational epitopes.

### Example 15.3

### Peptide Scan Technology

It is based on the synthesis of immobilized peptides on activated membranes using the Fmoc chemistry. Amino acid solutions are applied to the activated membrane leading to a peptide bond between the amino-group on the membrane (the membrane is activated with PEG) and the activated carboxy-group of the applied amino acid. After each cycle a specific washing procedure, acetylation, deprotection and monotoring of free amino-groups is performed. In contrast to the in vivo protein-synthesis membrane bound oligo-peptide chains are stepwise synthesized from C- to the N-terminus. Oligo-peptides containing natural as well as modified amino acids can be synthesized up to a length of 20 amino acids. Following synthesis the membranes are equilibrated and unspecific binding sites are blocked. After incubation with the antibody of interest and several washing steps the detection is performed using an HRP-conjugated secondary antibody in combination with the ECL-System. Membranes can be stripped, regenerated, and re-used up to 10 times depending on the antibody. Small overlapping oligo-peptides that ideally cover the complete amino acid sequence of the antigen of interest are synthesized on a solid support. This method allows the identification of linear epitopes on the amino acid level. It also allows rapid mutational studies.

### Example 16

### Inhibition of Tube Formation

HUVEC cells (Cell Line Service, Heidelberg; #0170 HU) were cultured in gelatine (0.2% gelatine in HBSS w/o Ca²⁺/Mg²⁺, 1 h at RT) coated 150 cm² flasks to 80-90% confluency. Only cells with passage numbers between 2 and 10 were used. An angiogenesis assay kit was used (Chemicon #ECM625) for the assay.

Materials:

96 well MTP, half well, tissue culture treated, black with clear bottom, Corning #3882

Trypsin for HUVEC cells, 0.25 mg/ml, Clonetics #CC-5012

Gelatine, 2%, Sigma #G1393

Biospin P6 columns, Biorad #7326200

Buffers:

PBS Dulbecco's, Invitrogen #14040-091

HBSS w/o Ca²⁺/Mg²⁺, Invitrogen #14170-088

Media:

Endothelial cell growth medium for HUVEC cells (CLS Heidelberg), containing: 2% FCS, 0.4% ECGS/H, 0.1 ng/ml EGF, 1.0 ng/ml bFGF, 1.0 μg/ml Hydrocortison, Gentamicin/Amphotericin B.

Purification of scFv's

ScFv's were purified with Biospin P6 columns (Biorad) to remove desthiobiotin, endotoxins and glycerol and were stored in PBS over night on ice. The protein concentration of the purified scFv was determined by measuring absorbance at 280 nm. All scFv solutions were adjusted to an identical concentration by addition of PBS.

Preparation of the MTP for the Tube Formation Assay

The required amount of buffer was added to the ECMatrix™ (both included in the angiogenesis kit). 25 μl diluted ECMatrix™ was added to each well of a pre-cooled 96 well MTP (half well plate). The MTP was incubated >1 h at 37° C. to allow the matrix solution to solidify. Detached endothelial cells were pre-incubated with the scFv's or appropriate IgG's for 30 min at RT.

Cell Seeding and Antibody Incubation

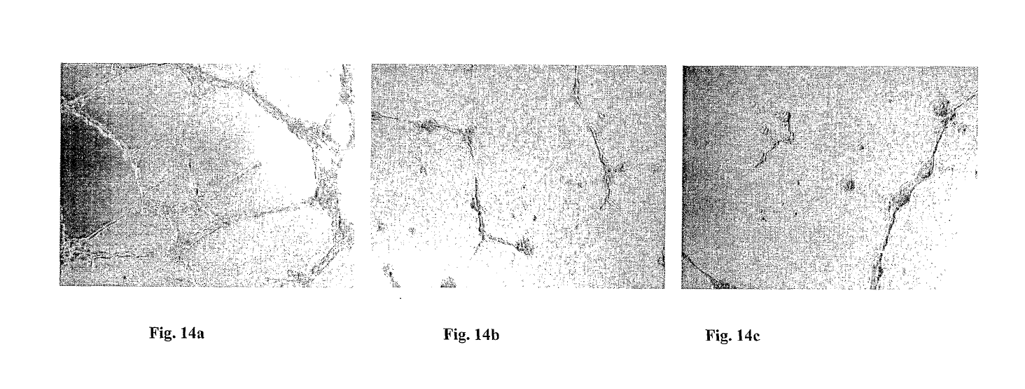

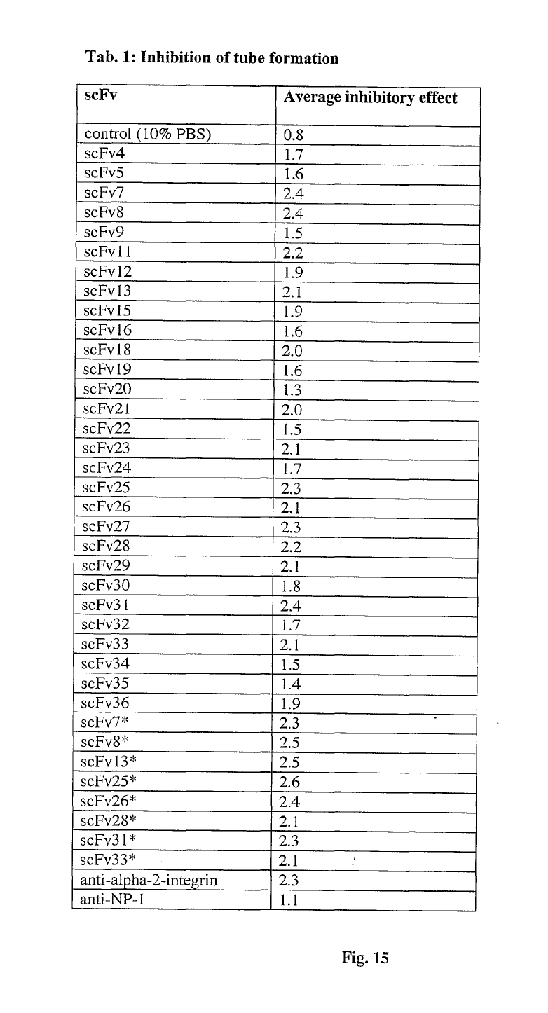

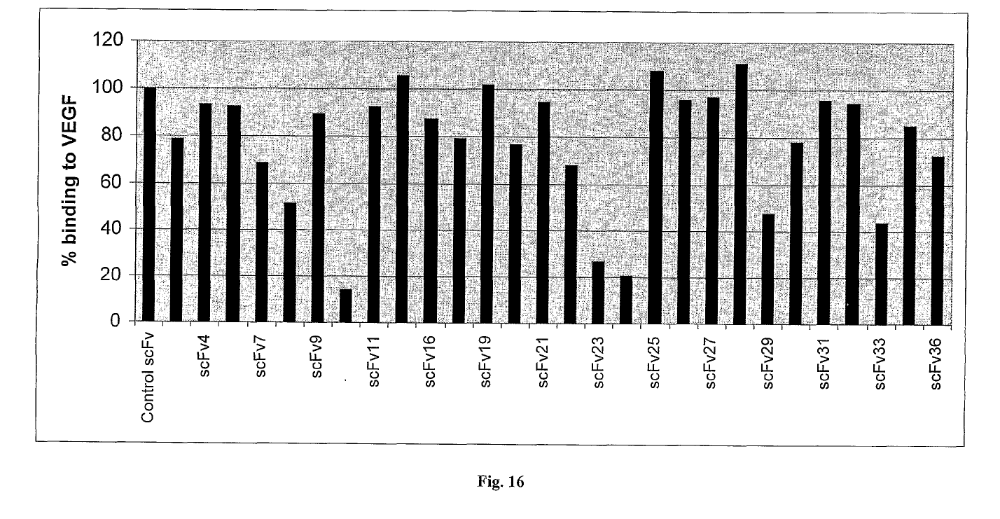

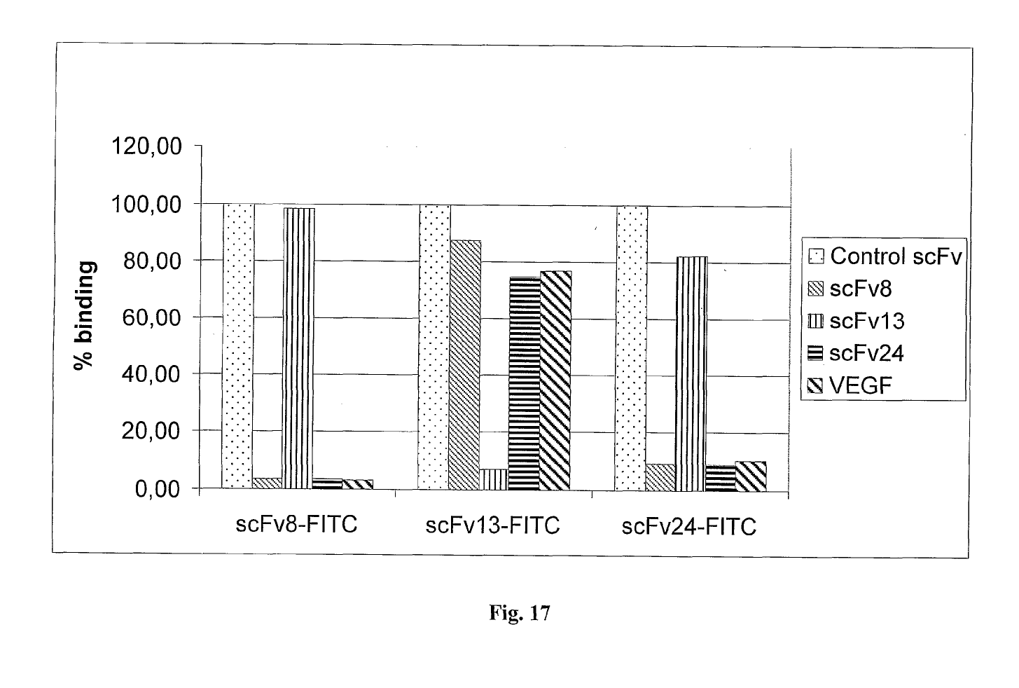

HUVEC cells were detached from the flask by removing cell culture medium, washing the cells lx with 10 ml HBSS w/o Ca²⁺/Mg²⁺ followed by addition of 5 ml trypsin (Clonetics) and incubation for 2-3 min at 37° C. Tiypsin reaction was stopped by adding 5 ml cell culture medium with FCS. Supernatants were combined, centrifuged for 5 min at 240×g, RT, the supernatant was carefully removed and the cells were resuspended in 5 ml medium and were counted by using a Neubauer cell counting chamber. A cell master mix was prepared by resuspending the required number of cells in the appropriate amount of cell culture medium to obtain a concentration of 10.000 cells in 50 μl per well after addition of the antibody solution. The end concentration in the well was usually 10 μg/ml for scFv's and 50 μg/ml for IgG's. For each binder a 3× premix was prepared. For each binder a 3× premix with 3× 10.000 cells was pre-incubated with the appropriate amount of scFv or IgG in a total volume of 150 μl for 30 min at RT before seeding, to allow binding to NP-1 before contact with matrix proteins. 50 μl of the cell-antibody mixture was transferred to each well of the MTP coated with 25 μl solidified ECMatrix. Appropriate controls were included in each experiment (PBS in same concentration as for samples with scFv, mouse IgG, commercial anti-NP-1 antibody (R & D Systems, #AF566), anti-α2 Integrin, as well as an unspecific control scFv's. Cells were incubated for 16 h at 37° C. The degree of tube formation was determined via light microscopy and was quantified based on the ability of the cells to form closed polygons, the number and area of the polygons, the number of branch points and the ability of the cells to form closed tubes e.g. connections between branch points. ScFv's were considered as positive if the ability to form closed polygons, the number of branch points and the ability to form closed connections between branch points were reduced. Inhibitory effects were quantified by comparison with the negative control and scaled between 0 and 3 (0-1=no inhibition and 2-3=strong inhibition effect). Several scFv were converted into an IgG1 format and were re-tested. FIG. 14a-c shows representative pictures of the tube formation assay. Several scFv showed significant inhibition of tube formation (Inhibition of >2.2). Table 1, as shown in FIG. 15 summarizes all results of the tube formation assay. Several scFvs inhibited the tube formation significantly, whereas the commercially available anti-NP-1 antibody did not show any significant inhibition of tube formation.

### Example 17

### Inhibition of NP-1/VEGF Interaction in ELISA

In order to test the ability of scFv's to inhibit the interaction between recombinant NP-1 and VEGF, VEGF₁₆₅ (R&Dsystems, #293-VE/CF) was coated at a concentration of 50 nM in PBS on Maxisorp microtiter plates (Nunc, #430341). Recombinant NP-1-Fc (R&Dsystems, #566-NNS) was preincubated at a concentration of 50 ng/ml with NP-1 scFv (25 μg/ml) for one hour at RT and was added to the VEGF₁₆₅ coated microtiter plate. Bound NP-1 was detected with anti-Fc-HRP (Jackson Immuno Research, #H909-035-098) followed by reaction with BM blue POD substrate (Roche, #11484281). The absorption was determined at 370 nM. Twenty-eight scFv's were tested for their ability to inhibit the interaction between recombinant NP-1 and VEGF₁₆₅. Twelve scFv's were found to significantly inhibit the interaction between NP-1 and VEGF₁₆₅. This result is compared to the results of the tube formation assay described in Example 16. scFv8 inhibits the interaction between NP-1 and VEGF₁₆₅ and inhibits HUVEC tube formation; scFv13 does not inhibit the interaction between NP-1 and VEGF₁₆₅ but inhibits HUVEC tube formation; scFv24 inhibits the interaction between NP-1 and VEGF₁₆₅ but does not inhibit HUVEC tube formation.

### Example 18

### Determination of NP-1 Epitopes

ScFv's were labeled with fluorescein isothiocyanate (Molecular Probes, #F-1906) according to the instructions of the manufacturer. HT1080 cells were preincubated to with a control scFv, a NP-1-binding scFv's (e.g. scFv8, scFv13 or scFv22) (both at 25 μg/ml) or VEGF₁₆₅ (R&Dsysterns, #293-VE/CF) (5 μg/ml) for 2 hours at 0° C. and the fluorescein labeled NP-1-binding scFv (5 μg/ml) was added for 30 min. at 0° C. Bound fluorescein labeled scFv were detected by flow cytometry and the geometric mean fluorescence intensity was recorded. In order to determine if NP-1-binding scFv has overlapping epitopes with VEGF and each other for binding to cell surface NP-1, a competition FACS with directly labeled scFv was performed. In agreement with the VEGF competition ELISA, scFv8 inhibits the interaction between NP-1 and VEGF₁₆₅ and inhibits HUVEC tube formation; scFv13 does not inhibit the interaction between NP-1 and VEGF₁₆₅ but inhibits HUVEC tube formation; scFv24 inhibits the interaction between NP-1 and VEGF₁₆₅ but does not inhibit HUVEC tube formation. Furthermore, scFv8 and scFv24 have mutually overlapping epitopes whereas scFv13 has an epitope distinct from scFv8 and scFv24.

### Example 19

### Transendothelial Invasion Assay with HT1080 Cells

Preparation of a Monolayer of Endothelial Cells:

A Neuroprobe membrane was coated with 50 μl/well 0.2% Gelatine (Sigma G-1393), was diluted in HBSS w/o Ca+/Mg+ ions (Gibco 14170-088) and was incubated for 1 h at RT under a fume hood. Confluent grown HUVEC cells were harvested with Trypsin (Cambrex CC-5012) and ECGM (CLS ready to use Endothelial cell Growth Medium) and were diluted with ECGM to 2×10⁵/ml. Remaining fluid on the coated membrane was removed with a syringe. The lower chamber of the Neuroprobe system was filled with 28.5 μl/well HBSS and the membrane was placed on top. 50 μl (1×10⁴) of the HUVEC cell suspension was spotted on the membranes in each well and was incubated for 48 h at 37° C. 5% CO₂.

Preparation of HT1080 Cells:

5×10⁵ cells were seeded into 6 150 cm² flasks and incubated for 48 h at 37° C. 5% CO₂.

Staining of HT1080 Cells:

Calcein AM (Molecular Probes C3099) was diluted 1:1000 in 90 ml DMEM with Glutamax that included 0.1% BSA (Sigma A-7030). The medium was removed from the HT1080 cells and 15 ml/flask of Calcein solution was added to the cells and cells were incubated for 15 min at 37° C. 5% CO₂. The solvent was removed and the cells were washed twice with HBSS w/o Ca+/Mg+ ions. Cells were harvested with EDTA and DMEM with Glutamax that included 0.1% BSA and were diluted to 7×10⁵/ml with DMEM with Glutamax that included 0.1% BSA.

Inhibition of Invasion of Cells:

All experiments were carried out in triplicates. 170 μl of the HT1080 cell suspension was pipetted into each well of a 96 well V-bottom plate. 17 μl scFv (min. 10 μg/ml), 0.25 mg/ml Cytochalasin D (CCD) (Sigma C-8273) was added and cells were incubated for 15 min. As a negative control a non-specific antibody was used.

| | | |
|---|---|---|
| Standardcurve: | 1 × 10⁴ cells | 46.8 μl cells + 133.2 μl DMEM with |
| | | Glutamax 0.1% BSA |
| | 2 × 10⁴ cells | 93.6 μl cells + 86.4 μl DMEM with Glu- |
| | | tamax 0.1% BSA |
| | 4 × 10⁴ cells | 57.2 μl cells directly spotted |

Preparation of the Membrane System for Invasion:

The membrane was disassembled from the lower chamber and the solvent was removed from the lower part of the membrane. The solvent from the wells of the lower chamber was removed as well. The lower chamber was filled with 30 μl/well ECGM/20% FCS (Invitrogen 10270106) for the test samples (row B-H). For the standard curve and for the background all wells of row A were filled with 30 μl/well ECM and membranes were placed on top.

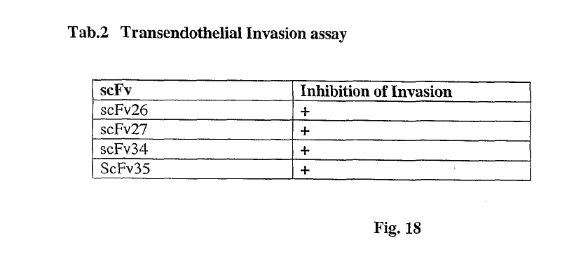

55 μl (3.5×10⁴) of HT1080 samples were pipetted on top of the HUVEC cells layer. The set up was incubated for 12-16 h at 37° C. and 5% CO₂. Cells from rows B-H were scraped off with a cell scraper and the membranes were rinsed with Dulbeccos PBS. Wells of rows B-H were wiped with a cotton swab (pre wetted in PBS) and membranes were rinsed again with PBS. The membranes were carefully dried and fluorescence at 485/520 nm was measured (Fluostar Galaxy). 4 different single chains inhibited the invasion by values of 6-10%. Table 2 as shown in FIG. 18 summarises the results.

### Example 20

### HUVEC Migration Assay

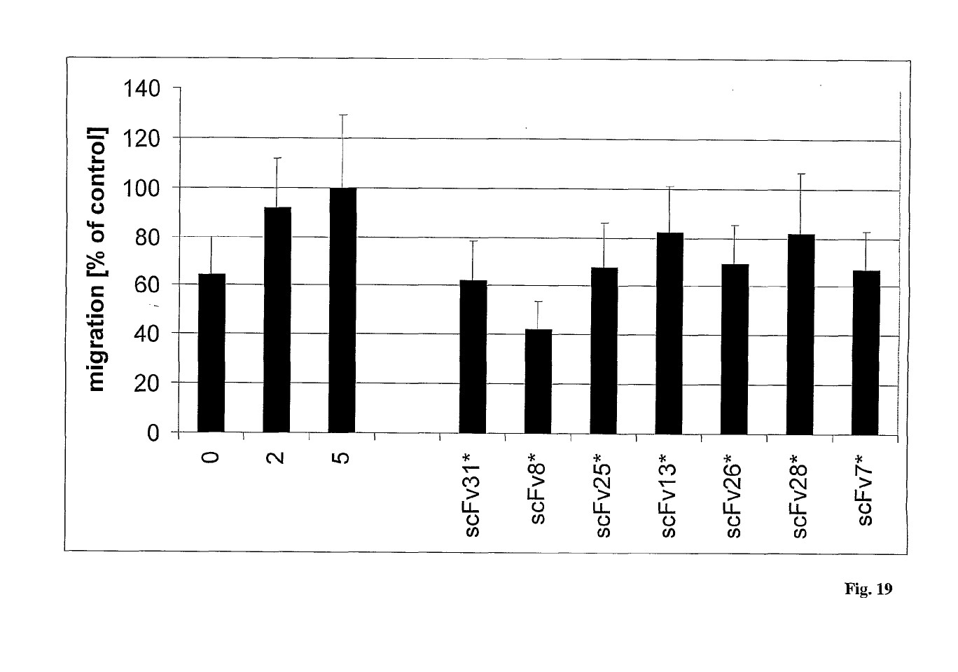

The HUVEC migration assay was carried out according to the manufacturer's instructions (BD Biosciences, Bedford/MA, Cat.-Nr. 354143). Briefly, 2×10⁴ HUVECs in 250 μl of basal medium (Promocell, Heidelberg, Germany, Cat-Nr. 22210) containing 1 ng/ml EGF, 1 μg/ml hydrocortisone, 0.4% FCS, 50 ng/ml amphothericin B and gentamicin 50 μg/ml (all from Cambrex, East Rutherford/NJ) were incubated with the antibodies (50 μg/ml) for 30 minutes at room temperature, then seeded into the top chambers of the 24-well insert, which contains a fibronectin-coated, 3-μm-pore FluoroBlok membrane at the bottom of each well. The lower chambers were filled with the same medium additionally containing 5 ng/ml VEGF₁₆₅ (R&D Systems, Minneapolis/Minn.). The plate was incubated for 22 h at 37° C., 5% CO₂, then the 24-well insert was transferred to a new plate containing 0.5 ml of a 4 μM solution of calcein-AM (Molecular Probes, Eugene/Oreg.) in HBSS (Invitrogen, Carlsbad/Calif.). After 90 minutes of incubation, the plate was read out in a BMG FluoStar plate reader (BMG LabTechnologies, Offenburg, Germany) at 485 nm excitation and 520 nm emission wavelength. Statistical analysis was done using a Kruskal-Wallis test, with significance assumed at p<0.05. Some results are shown in FIG. 19. As an example, 7 IgG's are shown in FIG. 19 that showed an inhibitory effect on the migration of HUVEC cells between 20-60%. scFv8*, scFv25*, scFv26*, and scFv31* showed an statistically relevant inhibitory effect. The dependency of the migration of HUVEC cells on the concentration of VEGF_{I65} is also shown in FIG. 19 (three bars on the left). Concentrations of 0-5 ng/ml were used to determine the concentration to dependency. The migration of HUVEC cells increased with increasing the VEGF₁₆₅ concentration.

1. A neuropilin binder (NPB) wherein the NPB is a polypeptide, antibody, scFv, antibody fragment or bioconjugate and is characterized in modulating neuropilin-1 (NP-1) function or having the capability to inhibit NP-1 dependent angiogenesis of endothelial cells and/or invasion of tumor cells, whereby the NPB binds to NP-1 and modulates NP-1 function.

2. The NPB according to claim 1 comprising a sequence selected from the group consisting of SEQ ID No: 1 or SEQ ID No: 2.

3. The NPB according to claim 1 comprising a sequence selected from the group consisting of sequences SEQ ID No: 5 to SEQ ID No: 38.

4. The NPB according to claim 3, whereby the NBP is not binding, interfering or inhibiting the VEGF/neuropilin-1 interaction.

5. The NPB according to claim 1, wherein the NPB is a polypeptide, antibody, scFv, antibody fragment or bioconjugate comprising one CDR3 (Complementary Determining Regions 3) having at least one of the sequences selected from SEQ ID No: 73 to SEQ ID No: 108.

6. The NPB according to any of claims 1 to 5, wherein said NPB is labeled with a detectable label; in particular wherein said detectable label is selected from the group consisting of a radioisotope, an enzyme and a chromophore.

7. Use of the NPB according to any of claims 1 to 6 for the detection of neuropilin-1 expression.

8. Use of the NPB according to any of claims 1 to 6 for the modulation of neuropilin-1 function, particularly modulation or inhibition of neuropilin-dependent invasion or adhesion of cells, preferably tumor cells.

9. A diagnostic kit comprising the NPB according to any of claims 1 to 6 and a container.

10. A composition comprising the NPB according to any of claims 1 to 6 and a pharmaceutical acceptable carrier.

11. An isolated nucleic acid molecule encoding the NPB according to any of claims 1 to 6.

12. A vector comprising anucleic acid according to claim 11.

13. Use of the NPB according to any of claims 1 to 6 in the manufacture of a medicament for the treatment or prevention of neuropilin-dependent angiogenesis and non-physiological blood vessel growth, particularly correlated with a tumor.

14. Use of the NPB according to any of claims 1 to 6 in the manufacture of a medicament for the treatment or prevention of cancer and/or metastasis of tumor cells, wherein the metastatic potential depends on neuropilin-1-related invasion and/or adhesion, preferably wherein the tumor cells are tumor cells derived from mesodermal cells.

15. The use of claim 13 or 14 wherein the NPB inhibits the function of expressed neuropilin-1, in particular wherein the molecule binds to the extracellular region of neuropilin-1.

16. An ex vivo method of determining the dependency of the invasiveness of a naturally occurring invasive cancer cell on the functionality of neuropilin-1, comprising the steps of:
b) contacting the cancer cell with a molecule inhibiting neuropilin-1 function;
c) contacting said cancer cell with a gel-like matrix under conditions suitable for the growth of said cancer cells; and
d) determining the migration of said cancer cells through the gel-like matrix.

17. An ex vivo method of determining the dependency of the adhesiveness of a naturally occurring invasive cancer cell on the functionality of neuropilin-1, comprising the steps of:
a) contacting the cancer cell with a molecule inhibiting neuropilin-1 function;
b) contacting said cancer cell with a layer of extracellular matrix (ECM) proteins under conditions suitable for the growth of said cancer cells; and
c) determining the adhesion of said cancer cells to the layer of ECM proteins.

18. The method of claim 17, wherein the layer of ECM proteins comprises one protein selected from the group consisting of collagen S type I, collagen VI, fibronectin, laminin, nidogen, entactin, and vitronectin.

19. A method of identifying a ligand binding specifically to the extracellular region of neuropilin-1, wherein said ligand is capable of inhibiting angiogenesis, tube formation of endothelial cells, and/or invasion or adhesion of tumor cells, comprising the steps of:
a) contacting a phage library of ligands with cancer cells or endothelial cells;
b) isolating said cells;
c) removing phages bound unspecifically and/or not bound to said cells;
d) eluting phages bound to said cells; and optionally
e) determining the identity of the ligand represented by said binding to neuropilin-1
f) testing the ligand in biochemical or biological assays on it capability to interfere with NP-1 function.

20. The method of claim 19, instead of steps e) further comprising the steps of:
e) contacting eluted phages with immobilized NP-1;
f) washing said NP-1 with detergent and/or high salt;
g) eluting phages bound to NP-1; and
h) determining the identity of the ligand represented by said eluted phages.

21. A method of treating or preventing cancer or metastasis in a patient, said method comprising administering to said patient the NBP according to any of claims 1 to 6, the composition according to claim 10, the nucleic acid sequence according to claim 11 or a ligand identifiable by the method of claim 19 or 20 in an amount effective to inhibit metastasis of neuropilin-1 mediated invasion and/or adhesion.

22. A method of treating or preventing cancer or metastasis in a patient, said method comprising administering to said patient the NBP according to any of the claims 1 to 6, the composition according to claim 10, the nucleic acid sequence according to claim 11 or a ligand identifiable by the method of claim 19 or 20 in an amount effective to inhibit tumor-associated, neuropilin-dependent angiogenesis.
